(19) United States
(12) Patent Application Publication
(10) Pub. No.: US 2025/0163388 A1
(43) Pub. Date: May 22, 2025

(54) METHODS FOR DERIVATION AND PROPAGATION OF AVIAN PLURIPOTENT STEM CELLS AND APPLICATIONS THEREOF

(71) Applicant: UNIVERSITY OF SOUTHERN CALIFORNIA, Los Angeles, CA (US)

(72) Inventors: Xi CHEN, Los Angeles, CA (US); Zheng GUO, Los Angeles, CA (US); Xinyi TONG, Los Angeles, CA (US); Qilong YING, Los Angeles, CA (US); Xugeng LIU, Los Angeles, CA (US)

(73) Assignee: UNIVERSITY OF SOUTHERN CALIFORNIA, Los Angeles, CA (US)

(21) Appl. No.: 18/835,222

(22) PCT Filed: Feb. 14, 2023
(86) PCT No.: PCT/US2023/012984

§ 371 Date: Aug. 1, 2024

Related U.S. Application Data
(60) Provisional application No. 63/310,464, filed on Feb. 15, 2022.
(60) Provisional application No. 63/311,003, filed on Feb. 16, 2022.

(51) Int. Cl. C12N 5/074; C12N 5/00; C12N 15/873

(52) U.S. Cl. CPC C12N 5/0696; C12N 5/0018; C12N 15/873; C12N 2500/24; C12N 2500/84; C12N 2501/235; C12N 2501/415; C12N 2501/602; C12N 2501/603; C12N 2501/605; C12N 2501/606; C12N 2501/608; C12N 2501/727; C12N 2506/02

(57) ABSTRACT

Disclosed herein are methods of deriving, producing, maintaining, or expanding embryonic stem cells (ESCs) from avian species and a culture medium used for such methods. According to various embodiments, the method includes culturing an embryo extracted from an avian egg in a culture medium to harvest cells from yolk of the avian egg; dissociating cells from the cultured embryo; isolating a morphologically undifferentiated ESC colony from the dissociated cells in a culture medium; and culturing the isolated ESC colony in the presence of ovotransferrin, thereby deriving ESCs. According to various embodiments, the culture medium includes a Wnt inhibitor; a protein kinase C (PKC) inhibitor; ovotransferrin; an inhibitor of activin receptor-like kinases-4, -5, and -7; and a leukemia inhibitory factor (LIF).

### CROSS-REFERENCE TO RELATED APPLICATIONS

This application claims benefit under 35 U.S.C. § 119(e) of U.S. Provisional Application Nos. 63/310,464 filed Feb. 15, 2022 and 63/311,003 filed Feb. 16, 2022, the contents of which are incorporated herein by reference in their entirety.

### FIELD

The present disclosure relates to development of conditions and culture formulations for derivation and propagation of embryonic stem cells (ESCs) from oviparous species, e.g., chickens and other avian species.

### BACKGROUND

Embryonic stem cells (ESCs) are derived from pluripotent epiblasts of the pre-implantation blastocysts and maintained in a culture condition to support their indefinite self-renewal in vitro. ESCs could be induced to differentiate into germline lineage and all three somatic germ layers, providing a useful platform to study cell differentiation in vitro. Importantly, upon in vivo transplantation, ESCs could efficiently integrate into the host and contribute to chimera formation. These chimeras will then pass genetic information to the next generation through germline transmission. This special property facilitates generation of genetically modified animals which have significantly pushed forward our understanding of biology in many aspects. To achieve the above functions, authentic ESCs should self-renew indefinitely in the dish, efficiently contribute to chimera formation in all three germ layers and differentiate into germ cells for germline transmission. However, despite their profound roles, authentic ESCs have only been derived from mouse and rat.

Application of chicken as a model system has a long and rewarding history, especially in developmental biology. Nevertheless, the advance of chicken system has been limited in recent years, mostly owing to the lack of transgenic models. The establishment of germline-competent chicken ESC lines could largely resolve this issue. Practically, the ease of acquiring fertilized chicken eggs from local farms facilitates our development of chicken ESC self-renewal culture condition. And previous studies have demonstrated the pluripotency of blastodermal cells from freshly laid chicken eggs (EGK.X stage) which give rise to both somatic and germline cells in ovo.

Although derivation of chicken ESCs has been developed for decades, authentic chicken ESC lines with germline competency have not been established yet. The frustration of maintaining germline competency in chicken ESCs to some extent leads to the hypothesis that germline specification is pre-determined by maternal inheritance in birds and cannot be induced from epiblast cells or in-vitro derived ESCs. Using previously reported culture conditions, the efficiency of chicken ESC derivation is relatively low. Putative ESC lines tend to gradually lose the typical ESC-like clonal morphology. Additionally, current culture formulations contain many complex parameters including serum medium and multiple growth factors. Derivation of ESC lines from other avian species has not been achieved based on these culture recipes. All the above observations suggest true chicken ESCs have not been established and whether chicken ESCs could contribute into germline lineage or not is still unknown.

Recent works on mouse pluripotent stem cells have defined pluripotency into multiple states. Interestingly, even pluripotent cells from closely related development stages show very distinct responses to signaling-pathway manipulation. Because the exact pluripotent state for avian blastodermal cells is undetermined, what is needed is identification of an effective culture condition from small molecules and cytokines involved in regulating a broad range of pluripotent states. The effective culture condition should not be limited by previous conditions established for mouse pluripotent cell derivation. In the present disclosure, we show a defined medium and a method supporting efficient derivation and maintenance of true ESCs fromoviparous species, e.g., chicken and other avian species.

### BRIEF DESCRIPTION OF THE DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIGS. 1A-1F depict dual inhibition of Wnt signaling and PKC supporting the derivation and long-term maintenance of chicken ESCs in combination with ovotransferrin. FIG. 1A) A representative image of chicken ESCs maintained in IWR1+Gö6983+3% yolk at passage 35 (top panel, scale bar, 100 μm). The bottom panel image is a zoom-in view of the dotted area of the top panel image (scale bar, 50 μm). FIG. 1B) Schematic showing the strategy for the identification of the functional component(s) in egg yolk that promotes chicken ESC self-renewal. FIG. 1C) SDS-PAGE analysis of fractions of ammonium sulfate precipitates. Whole: yolk plasma mixtures within the range of 50 kDa to 100 kDa MWCO. Dotted squares indicate gels cut for Mass-spec analysis. FIG. 1D) List of the top 10 peptides from 80p identified by Mass-spec analysis and comparison with peptide coverage in 50p and 70p. FIG. 1E) A representative image of chicken ESCs maintained in IWR+Gö6983+ovotransferrin at passage 25. Scale bar, 50 μm. FIG. 1F depicts a table of Mass-spec results of 50p, 70p, and 80p.

FIGS. 2A-2C depict ESCs derived from multiple avian species. FIG. 2A) Representative images of ESCs derived from multiple avian species in OT/3i/chLIF. Scale bar, 50 μm. FIG. 2B) A representative image of ostrich ESCs derived and maintained in OT/3i. Scale bar, 50 μm. FIG. 2C) Growth curve of a representative chicken ESC line (RIR41) cultured in OT/2i and OT/3i/chLIF conditions.

FIGS. 3A-3B depict in vitro characterization of chicken ESCs. FIG. 3A) qRT-PCR analysis of the relative expression of lineage marker genes in embryoid bodies formed from chicken ESCs. Data represent means±SD of three biological replicates. FIG. 3B) Immunostaining of cells differentiated from chicken ESCs. Scale bar, 50 μm.

FIGS. 4A-4G depict transcriptomic characterization of chicken ESCs. FIG. 4A) PCA analysis of RNA-seq profiles for chicken ESCs and cells isolated from EGK.VIII, EGK.X and HH4 stage chicken embryos. FIG. 4B) Gene set enrichment analysis showing enrichment score of EGK.X and HH4 signature in chicken ESCs respectively. NES, normalized enrichment score. FIG. 4C) Scatter plot showing gene expression difference between chicken ESCs/EGK.X and chicken ESCs/HH4. FIG. 4D) Expression level (FPKM) of selective pluripotent genes in chicken ESCs, EGK.X and HH4 stage embryos. FIG. 4E) Gene Ontology analysis of successfully downregulated genes in chicken ESCs. Successfully downregulated genes meaning genes were expressed at a lower level in both chicken ESCs and EGK.X in comparison to HH4. FIG. 4F) RNA expression levels of the successfully downregulated genes (top panel) and differentiation-related genes (bottom panel, from GO term cluster) displayed by Violin plots. FIG. 4G) Transcriptome correlation analysis across species. Dotted box highlights the comparison of chicken ESCs with various states of primary chicken cells or mouse pluripotent stem cells.

FIGS. 5A-5AB demonstrate that avian ESCs can contribute to chimera formation both ex-ovo and in ovo. FIG. 5A) GFP-labeled chicken ESCs plated onto EGK.X chick embryo. Ex-ovo embryo development was traced for 4 days. Scale bar, 400 μm. FIG. 5B) Left panel, images of GFP-labeled chicken ESCs (cESCs). Scale bars, 100 μm. Right panel, GFP-labeled cESCs plated onto HH4 chick embryo. Ex-ovo embryo development was traced for 2 days. Scale bar, 400 μm. FIG. 5C) An image of the surrogate open-shell system for chimeric embryo development in ovo. FIG. 5D) Representative images showing GFP-labeled donor cells inside irradiated chick embryos at E5. Scale bar, 400 μm. FIG. 5E) A representative image showing contribution of GFP-labeled donor cells in both somatic and extraembryonic lineages in a E4 chimeric embryo. Scale bar, 400 μm. FIG. 5F) An image showing GFP-labeled donor cESCs contribute to chick extraembryonic blood vessels. Scale bar, 100 μm. FIG. 5G) Images showing feather color of control, low-, medium- and high-grade chimeric embryos at E20. Donor ESCs were derived from Rhode Island Red; Recipient embryo, White Leghorn. FIG. 5H) Images showing GFP-labeled donor cells inside the indicated organs. E13 heart; E20 tongue; E8 midbrain; E7 stomach; E21 liver; E19 intestine. FIG. 5I) Top panel: GFP-labeled goose ESCs were injected into the subgerminal cavity of EGK.X chick embryo and incubated for 4 days. Right, enlargement of square image showing GFP-labeled donor goose cells inside E4 chick embryo. Bottom panel: GFP-labeled quail ESCs were injected into the subgerminal cavity of EGK.X chick embryos and incubated for 4 (left) or 19 days (right). FIG. 5J) E8 quail-quail chimera. FIGS. 5K-5AB) depict a summary of chimeric embryos generated from avaian ESCs.

FIGS. 6A-6G demonstrate that avian ESCs can give rise to germ cells both in vitro and in ovo. FIG. 6A) qRT-PCR analysis of the expression of germ cell markers in chicken ESC-derived embryoid bodies formed in N2B27 medium. Data represent means±SD of three biological replicates. FIG. 6B) Immunofluorescence DAZL staining of chicken ESCs maintained in OT/3i/chLIF. Scale bar, 50 μm. FIG. 6C) Immunofluorescence DAZL staining of day-3 chicken and goose ESC-derived embryoid bodies. Scale bar, 10 μm. FIG. 6D) GFP fluorescence images of testes in a E15 high-grade chicken ESC/embryo chimera. Scale bar, 50 μm. FIG. 6E) Sections from chimeric gonads at E9 and E15 chicken ESC/embryo chimeras stained for GFP and DAZL antibodies. E9, scale bar 10 μm. E15, scale bar, 50 μm. FIG. 6F) Sections from chimeric gonads at E15 double-stained for GFP/SSEA1-1 antibodies. Scale bar, 10 μm. FIG. 6G) Sections from chimeric gonads at E21 triple-stained for GFP/P63/SOX9 or GFP/DAZL/SOX9 antibodies. Top row, arrows indicate GFP+/P63+/SOX9− germ cells (labeled as GC). Bottom row, arrows indicate GFP+/P63−/SOX9+ Sertoli cells (labeled as SC). Scale bar, 20 μm.

FIGS. 7A-7M demonstrate the contribution of chicken ESCs to feather patterning. FIG. 7A) Morphology of contour feather from adult white leghorn chicken. FIG. 7B) H&E staining of feather follicle from adult white leghorn chicken. FIG. 7C) Bright view of feather follicle from adult white leghorn chicken. FIG. 7D) MITF staining for melanocyte progenitor cells from adult white leghorn chicken. FIG. 7E) Morphology of contour feather from adult Rhode Island red chicken. FIG. 7F) H&E staining of feather follicle from adult Rhode Island red chicken. FIG. 7G) Bright view of feather follicle from adult Rhode Island red chicken. FIG. 7H) MITF staining for melanocyte progenitor cells from adult Rhode Island red chicken. FIG. 7I) Morphology of contour feather from adult chimeric chicken. FIG. 7J) H&E staining of feather follicle from adult chimeric chicken. FIG. 7K) Bright view of feather follicle from adult chimeric chicken. FIG. 7L) MITF staining for melanocyte progenitor cells from adult chimeric chicken. FIG. 7M) GFP fluorescence of adult chimeric feather.

FIGS. 8A-8J demonstrate that egg yolk supports chicken ESC self-renewal. FIG. 8A) Representative images of chicken blastodermal cells cultured in the indicated conditions for 3 days. Scale bar, 50 μm. FIG. 8B) Representative images of chicken blastodermal cells cultured in IWR+Gö6983 for 3 days. Right image, Scale bar, 100 μm. Left image is a zoom-in view of the dotted area of the right image; Scale bars, 50 μm. FIG. 8C) Detection of Nanog mRNA expression by RNAscope in situ hybridization in chicken blastodermal cells cultured in the indicated conditions for 3 days. Scale bar, 100 μm. FIG. 8D) Representative images of chicken blastodermal cells cultured in the indicated conditions after passaging. Scale bar, 50 μm. FIG. 8E) A representative image of chicken blastodermal cells cultured in IWR+Gö6983 for 10 days without passaging. Scale bar, 50 μm. FIG. 8F) A representative image of chicken blastodermal cells cultured in IWR+Gö6983 supplemented with 3% yolk collected from fertile egg. Scale bar, 50 μm. FIG. 8G) Representative images of chicken ESCs passaged with or without the indicated supplements. NT, non-treatment control. Scale bar, 50 μm. FIG. 8H) Representative images of chicken ESCs cultures supplemented with indicated yolk fractions. Scale bar, 50 μm. FIG. 8I) Representative images of chicken ESCs maintained in IWR+Gö6983+ovotransferrin. BPR, Barred Plymouth Rock, cells at p14. WL, White Leghorn, cells at p15. Scale bar, 50 μm. FIG. 8J) Representative images of chicken ESCs cultured with or without the indicated supplements. NT, non-treatment control. Scale bar, 50 μm.

FIGS. 9A-9E depict derivation of ESCs from multiple avian species. FIG. 9A) Immunofluorescence staining of pheasant blastodermal cells cultured in OT/2i. Scale bar, 50 μm. FIG. 9B) Quail blastodermal cells cultured in OT/2i with or without the indicated supplements. Scale bar, 50 μm. FIG. 9C) Representative images of quail ESCs maintained in the indicated conditions for 3 passages. Scale bar, 50 μm. FIG. 9D) Representative images of ostrich ESCs maintained in the indicated conditions. Scale bar, 50 μm. FIG. 9E) Phase contrast images of HH2-HH5 chicken embryonic cells cultured in the OT/3i/chLIF. Scale bar, 50 μm.

FIGS. 10A-10D depict gene expression analysis of chicken ESCs and cells isolated from different developmental stages of chick embryos. FIG. 10A) Heatmap comparison of chicken ESCs with chicken primary cells isolated from EGK.I to HH4 stage embryos. (EGK.I to VIII data were from accession number GSE86592; HH4 data from GenBank:SRX893876). FIG. 10B) FPKM scatter plots with Pearson correlations showing a higher linear correlation between chicken ESCs and EGK.X (R=0.8). FIG. 10C) Gene Ontology analysis of mis-upregulated genes in chicken ESCs. The term Mis-upregulated means genes mis-expressed at a higher level in chicken ESCs and those genes were not differentially expressed in EGK.X and HH4 stages. FIG. 10D) RNA expression levels of the mis-upregulated genes displayed by Violin plots.

FIGS. 11A-11F demonstrate that avian ESCs can contribute to chimera formation. FIG. 11A) Surrogate open-shell system used for the injection of chicken ESCs into chick embryos. FIG. 11B) sequential images showing the development and hatchling of a chick embryo injected with chicken ESCs using the surrogate open-shell system. FIG. 11C) Representative images showing biased lineage contribution in low-rate chicken ESC/embryo chimera. Left, E7 chimera. Right, E6 chimera. FIG. 11D) A representative image of E4 chimeric chick embryo injected with GFP donor chicken ESCs at p39. Scale bar, 400 μm. FIG. 11E) GFP-labeled quail ESCs (left panel, scale bar, 50 μm) were injected into EGK.X chick embryos and the contribution of quail ESC-derived cells in the chick embryos was traced for 3 days. Scale bar, 220 μm. FIG. 11F) GFP-labeled goose ESCs (left panel, scale bar, 50 μm) were injected into EGK.X chick embryos and the contribution of goose ESC-derived cells in the chick embryos was traced for 3 days. Scale bar, 220 μm.

FIGS. 12A-12D depict authentic ESCs derived from different avian species. FIG. 12A. Representative images of ESCs derived from multiple avian species. Scale bar, 25 μm. FIG. 12B. Chicken chimeric embryo generated by injection of GFP-labeled chicken ESCs into the EGK X stage chicken embryo. FIG. 12C. Images showing feather color of control, low-, medium- and high-grade chimeric embryos at embryonic day 20 (E20). The rightmost two images show the same live-hatched male chimeric chick at one week (left) and 18 months (right). Donor ESCs were derived from Rhode Island Red chicken (with black feathers). The recipient embryos are White Leghorn (with white feathers). FIG. 12D. Quail and goose ESCs can also form interspecies chimeras when injected into chicken embryos. GFP-labeled quail or goose ESCs were injected into EGK-X chick embryos and the contribution of quail or goose ESC-derived cells in the ex ovo-incubated chick embryos was tracked for 3 days.

FIGS. 13A-13D demonstrate that chicken ESCs can differentiate into germ cells in vitro. FIG. 13A. qRT-PCR analysis of the expression of germ cell markers in chicken ESC-derived EBs. Data represent means±SD of three biological replicates. FIGS. 13B-13D. DAZL immunostaining of undifferentiated chicken ESCs (FIG. 13B), day-4 chicken and goose ESC-derived EBs (FIG. 13C), and day-4 EBs derived from chicken ESCs engineered to express an inducible chicken Nanog transgene (FIG. 13D). It was demonstrated that inducible expression of chicken Nanog can significantly increase chicken ESC differentiation efficiency towards germ cells.

FIGS. 14A-14D demonstrate that chicken ESCs contribute to germ cells in ovo. FIG. 13A. GFP fluorescence images of testes in a E15 high-grade chicken ESC/embryo chimera. Scale bar, 50 μm. FIG. 13B. Sections of E9 and E15 chimeric gonads stained for GFP and DAZL antibodies. E9, scale bar 10 μm. E15, scale bar, 50 μm. FIG. 13C. Sections of E15 chimeric gonads double-stained for GFP/SSEA1-1 antibodies. Scale bar, 10 μm. FIG. 13D. Sections of E21 chimeric gonads triple-stained for GFP/P63/SOX9 or GFP/DAZL/SOX9 antibodies. Top panel, arrows indicate GFP+/P63+/SOX9-germ cells (labeled as GC). Bottom panel, arrows indicate GFP+/P63−/SOX9+Sertoli cells (labeled as SC). Scale bar, 20 μm.

FIGS. 15A-15D depict germ cell reporter lines. FIGS. 15A and 15C. Diagrams showing the gene-targeting strategies to generate Dazl-Cerulean (FIG. 15A) and Nanog-mCherry (FIG. 15C) knockin reporter lines. FIG. 15B. Dazl-Cerulean gene-targeted chicken ESCs before (top panel) and after (bottom panel) Cre-mediated recombination to remove the CAG-mCherry cassette. FIG. 15D. Nanog-mCherry gene-targeted chicken ESCs. These ESCs express a GFP transgene.

FIGS. 16A-16F depict chicken iPSC derivation. FIG. 16A. Retroviral vector is highly efficient in introducing GFP transgene into CEFs. FIG. 16B. A representative iPSC colony generated from MEFs transfected with mouse OSKM. FIG. 16C. No iPSC colony emerged in MEFs transfected with chicken OSKM. FIG. 16D. CEFs transfected with mouse OSKM. FIG. 16E. CEFs transfected with chicken OSKM. FIG. 16F. Chicken iPSCs spontaneously differentiate into neurons under serum-free conditions.

### DETAILED DESCRIPTION

As described herein, prior art methods did not permit cell culture of oviparous (e.g., avian) ESC lines, or maintenance of ESCs in culture. The inventors have developed methods and combinations of reagents that permit derivation, production, maintenance, and expansion of oviparous (e.g., avian) stem cells (e.g., ESCs or iPSCs).

Accordingly, in one aspect, described herein is a method of producing, enriching, maintaining, or expanding oviparous stemcells (SCs), the method comprising contacting at least one oviparous stem cell or oviparous blastodermal cell with at least one of: a Wnt inhibitor; a protein kinase C (PKC) inhibitor; and ovotransferrin.

As used herein, the term “expanded population” of stem cells refers to a population of cells comprising at least one more stem cell, such that the quantity of stem cells in the population is greater (e.g., at least 10% greater, at least 20% greater, at least 30% greater) than the number of stem cells prior to contacting with one or more agents as described herein (e.g., a Wnt inhibitor; a protein kinase C (PKC) inhibitor; and/or ovotransferrin).

As used herein, “contacting” one or more cells with one or more agents can be achieved in a variety of ways. For instance, a population of cells may be contacted with one or more agents by culturing the cells in the presence of these agents for a period of time, such as for two or more days. When a cell(s) are contacted with more than one agent, the agents can be present in the cell culture medium together, such that the cells are exposed to the one or more agents simultaneously. Alternatively, the one or more agents may be added to the cell culture medium sequentially. For instance, the one or more agents may be added to a population of cells in culture according to a particular regimen, e.g., such that different agents are added to the culture media at different times during a culture period.

As used herein, a “cell culture” refers to an in vitro population of cells having a population of metabolically active cells. The number of these cells can be roughly stable over a period of at least 3 days or can grow. As used herein, “culturing” refers to continuing the viability of a cell or population of cells. In some embodiments of any of the aspects, the phenotype, morphology, number, or differentiation status of the cultured cells can change over time. In some embodiments of any of the aspects, the phenotype, morphology, or differentiation status of the cultured cells does not change over time. Conditions suitable for cell culture for different cell types are well known in the art and cell culture media for various cell types is readily available. Exemplary media and conditions are provided elsewhere herein. Culturing refers to maintaining a cell culture overtime and can comprise contacting the culture with appropriate media and/or providing appropriate environmental conditions (such as temperature and humidity). The appropriate conditions and media will vary depending on cell type selected and selection of the appropriate conditions and media is well within the ordinary skill in the art, e.g., utilizing a commercially available media advertised for that cell type. Culturing can be performed in static or flowing media and can comprise changing the media at intervals or continuously.

In some embodiments of any of the aspects, the method described herein produces germline competent and pluripotent stem cells, e.g., ESCs. In some embodiments of any of the aspects, the method described herein maintains germline competent and pluripotent stem cells, e.g., ESCs. In some embodiments of any of the aspects, the method described herein expands germline competent and pluripotent stem cells, e.g., ESCs.

In some embodiments of any of the aspects, the method described herein produces morphologically undifferentiated stem cells, e.g., ESCs. In some embodiments of any of the aspects, the method described herein maintains morphologically undifferentiated stem cells, e.g., ESCs. In some embodiments of any of the aspects, the method described herein expands morphologically undifferentiated stem cells, e.g., ESCs.

In some embodiments of any of the aspects, the method described herein produces stem cells, e.g., ESCs, expressing Nanog mRNA. In some embodiments of any of the aspects, the method described herein maintains stem cells, e.g., ESCs, expressing Nanog mRNA. In some embodiments of any of the aspects, the method described herein expands stem cells, e.g., ESCs, expressing Nanog mRNA.

As used herein, “oviparous” refers to an animal that lays eggs. Oviparous animals include, as non-limiting examples, fish, amphibians, birds, monotremes, and most reptiles, In some embodiments of any of the aspects, an oviparous animal is a bird and/or an oviparous cell described herein (e.g., an oviparous stem cell) is an avian cell. In some embodiments of any of the aspects, an oviparous animal is a fish and/or an oviparous cell described herein (e.g., an oviparous stem cell) is a fish cell. In some embodiments of any of the aspects, an oviparous animal is a reptile and/or an oviparous cell described herein (e.g., an oviparous stem cell) is a reptile cell. In some embodiments of any of the aspects, an oviparous animal is an amphibian and/or an oviparous cell described herein (e.g., an oviparous stem cell) is an amphibian cell. In some embodiments of any of the aspects, an oviparous animal is a monotreme and/or an oviparous cell described herein (e.g., an oviparous stem cell) is a monotrme cell.

In some embodiments of any of the aspects, a fish can be Danio rerio (zebrafish). In some embodiments of any of the aspects, a fish can be Oreochromis niloticus (Nile tilapia), Oreochromis aureus (Blue tilapia), Oreochromis mossambicus (Mozambique tilapia), and/or hybrids thereof. In some embodiments of any of the aspects, a fish can be Morone saxatilis (striped bass) and hybrids thereof. In some embodiments of any of the aspects, a fish can be Lates calcarifer (barramundi). In some embodiments of any of the aspects, a fish can be Perca flavescens (yellow perch). In some embodiments of any of the aspects, a fish can be an Aceipenseridae (sturgeon). In some embodiments of any of the aspects, a fish can be Sander vitreus (walleye). In some embodiments of any of the aspects, a fish can be a trout, e.g, a Salmoninae species. In some embodiments of any of the aspects, a fish can be a salmon, e.g., a Salmonidae species. In some embodiments of any of the aspects, a fish can be a catfish, e.g., a Siluriformes or Nemtognathi species. In some embodiments of any of the aspects, a fish can be Lepomis macrochirus (bluegill). In some embodiments of any of the aspects, a fish can be Micropterus salmoides (largemouth bass). In some embodiments of any of the aspects, a fish can be a shiner, e.g., a Notropis species (Eastern shiners), a Lythrurus species (Finescale shiners), a Pteronotropis species (e.g., Flagfin shiners), a Notemigonus crysoleucas (Golden shiner), a Luxilus species (Highscale shiners), a Richardsonius species (Redside shiners), or a Cyprinella species (Satinfin shiner). In some embodiments of any of the aspects, a fish can be a sunfish, e.g., a Centrarchidae species or Lepomis species. In some embodiments of any of the aspects, a fish can be a koi or carp, e.g., a Cyprinidae species, e.g., Ctenopharyngodon idella (grass carp), Hypophthalmichthys molitrix (silver carp), Cyprinus catla (Indian carp), Cyprinus carpio (Common carp), Hypophthalmichthys nobilis (Bighead carp), Carassius carassius (Crucian carp), Labeo rohita (Roho carp), and the like.

In some embodiments of any of the aspects, the at least one oviparous (e.g, avian) stem cell or oviparous (e.g, avian) blastodermal cell is contacted with a Wnt inhibitor and a PKC inhibitor. In some embodiments of any of the aspects, the at least one oviparous (e.g, avian) stem cell or oviparous (e.g, avian) blastodermal cell is contacted with a Wnt inhibitor and ovotransferrin. In some embodiments of any of the aspects, the at least one oviparous (e.g, avian) stem cell or oviparous (e.g, avian) blastodermal cell is contacted with a Wnt inhibitor and ovotransferrin. In some embodiments of any of the aspects, the at least one oviparous (e.g, avian) stem cell or oviparous (e.g, avian) blastodermal cell is contacted with a Wnt inhibitor, a PKC inhibitor, and ovotransferrin.

The hallmark of canonical Wnt signaling activation is elevated levels of the protein β-catenin. β-catenin is constitutively produced and is present in the cytoplasm as pools of monomeric protein. The primary mechanism for controlling cytoplasmic levels of β-catenin is through direct physical degradation upon recruitment into a large multi-protein complex (“degradation complex”). After formation, the complex is stabilized by the GSK3β-mediated phosphorylation of the protein components Axin and APC, as well as PP2A. GSK3β-then phosphorylates β-catenin, thereby allowing it to be recognized by β-transducin repeat containing protein (β-TrCP), and targeting it for ubiquitination and proteosomal degradation. An alternative degradation pathway has been shown involving ubiquitination induced by complexation with Siah-1 and the C-terminus of APC. β-catenin can be found at the cell surface sites of intercellular contact known as adherens junctions, where it is complexed with E-cadherin. Thus, the breakdown of the E-cadherin-catenin complex can increase cytoplasmic levels of free β-catenin, thereby stimulating transcriptional activity. Thus, activation of the cell surface receptors cRON, epidermal growth factor receptor (EGFR) and c-ErbB2, by liberating 3-catenin, can also stimulate canonical Wnt signaling. Other signaling pathways can either activate or facilitate the effects of Wnt signaling.

As used herein, the term “Wnt antagonist” or “Wnt inhibitor” refers to any agent that inhibits the Wnt/β-catenin pathway, or enhances the activity and/or expression of inhibitors of Wnt/β-catenin signaling, for example activators or enhancers of GSK-3β activity. A Wnt inhibitory agent as used herein can suppress the Wnt/β-catenin pathway at any point along the pathway, for example, but not limited to decreasing the expression and/or activity of Wnt, or β-catenin or Wnt dependent genes and/or proteins, and increasing the expression and/or activity of endogenous inhibitors of Wnt and/or β-catenin or increasing the expression and/or activity of endogenous inhibitors of components of the Wnt/β-catenin pathway, for example increasing the expression of GSK-3β.

Exemplary methods that can be used to determine the activity of a Wnt inhibitor include, without limitation, monitoring the expression of a reporter gene under the control of a TCF/LEF family transcription factor, as well as TOPFlash luciferase reporter assays, as described in US 2014/0044763.

Inhibitors of this pathway are known in the art and include IWR-1 (also known in the art as IWR-1-endo), E7449, AZ6102, WIKI4, 53AH, C59, XAV-939, ICG-001, triptonide, IWP-2, M2912 (MSC2504877), CCT251545, KY02111, M2912, Box5, KY05009, Heparan sulfate, Tetramethylthiuram disulfide (TMTD), JW55, MN64, RK-287107, G007-LK, adavivint (SM04690). In some embodiments of any of the aspects, the Wnt inhibitor is IWR-1, e.g., CAS No. 1127442-82-3. In some embodiments of any of the aspects, the Wnt inhibitor is IWR-1, XAV939, 53AH, or C59.

“Protein kinase C” or “PKC” refers to a family of protein kinases that phosphorylate serine and threonine residues on target proteins. PKC inhibitors are known in the art and include Go6983 (also referred to herein as “Gö6983”), Go7874 (also referred to herein as “G67874”), Go6976 (also referred to herein as “G66976”), LY317615, GSK590693, HA-1077, Ro320432, Calophostin C, AEB071 (sotrastaurin), NSC-301739 (mitoxantrone), AM-2282 (staurosporine), GF109203X (bisindolylmaleimide I), Ro 31-8220 (bisindolylmaleimide IX), daphnetin, dequalinium chlrodie, A-3 hydrochloride, N-desmethyltamoxifen, bisindolylmaleimide VIII, H-1152, HA-100, rottlerin, bisindolylmaleimide IV, VTX-27, valrubicin, PKC-theta inhibitor, ML-7, LY333531, PKC412, NSC 646662, WAY-301398, epsilon-VI-2, chelerythrine, hypocrellin A, hypericin, oncrasin-1, LXS-196, NAS-301, 2-methoxy-1,4-naphthoquinone, quercetin, myricitrin, and methyl-hesperidin. In some embodiments of any of the aspects, the PKC inhibitor is G06983 or G06976. In some embodiments of any of the aspects, the PKC inhibitor is G06983.

Further PKC inhibitors and Wnt inhibitors are known in the art and readily identified by one of skill in the art, e.g., in the bindingdb.org database.

As described herein, the inventors have recognized that maintenance of stem cells, e.g., expanding or culturing stem cells in vitro requires a transferrin that is appropriately matched to the origin of the stem cell. That is, mammalian transferrin cannot support maintenance, expansion, or culture of oviparous stem cells—rather, an oviparous ovotransferrin is required. Species-matched ovotransferrin can provide even more effective results, e.g., when the cell and the ovotransferrin originate from the same species. Accordingly, in some embodiments of any of the aspects, an oviparous stem cell of a first oviparous species or oviparous blastodermal cell of a first ovirparous species is contacted with ovotransferrin of the first oviparous species. In some embodiments of any of the aspects, an oviparous stem cell of a first oviparous species or oviparous blastodermal cell of a first oviparous species is contacted with ovotransferrin comprising a naturally occurring ovotransferrin sequence found in the first oviparous species.

In some embodiments of any of the aspects, the ovotransferrin has a sequence with at least 70% sequence homology to an endogenous ovotransferrin encoded in a genome of the at least one oviparous stem cell or oviparous blastodermal cell. In some embodiments of any of the aspects, the ovotransferrin has a sequence with at least 80% sequence homology to an endogenous ovotransferrin encoded in a genome of the at least one oviparous stem cell or oviparous blastodermal cell. In some embodiments of any of the aspects, the ovotransferrin has a sequence with at least 85% sequence homology to an endogenous ovotransferrin encoded in a genome of the at least one oviparous stem cell or oviparous blastodermal cell. In some embodiments of any of the aspects, the ovotransferrin has a sequence with at least 90% sequence homology to an endogenous ovotransferrin encoded in a genome of the at least one oviparous stem cell or oviparous blastodermal cell. In some embodiments of any of the aspects, the ovotransferrin has a sequence with at least 95% sequence homology to an endogenous ovotransferrin encoded in a genome of the at least one oviparous stem cell or oviparous blastodermal cell.

In some embodiments of any of the aspects, the ovotransferrin has a sequence with at least 80% sequence identity to an endogenous ovotransferrin encoded in a genome of the at least one oviparous stem cell or oviparous blastodermal cell. In some embodiments of any of the aspects, the ovotransferrin has a sequence with at least 85% sequence identity to an endogenous ovotransferrin encoded in a genome of the at least one oviparous stem cell or oviparous blastodermal cell. In some embodiments of any of the aspects, the ovotransferrin has a sequence with at least 90% sequence identity to an endogenous ovotransferrin encoded in a genome of the at least one oviparous stem cell or oviparous blastodermal cell. In some embodiments of any of the aspects, the ovotransferrin has a sequence with at least 95% sequence identity to an endogenous ovotransferrin encoded in a genome of the at least one oviparous stem cell or oviparous blastodermal cell. In some embodiments of any of the aspects, the ovotransferrin has a sequence identical to an endogenous ovotransferrin encoded in a genome of the at least one oviparous stem cell or oviparous blastodermal cell.

In some embodiments of any of the aspects, an oviparous stem cell of a first oviparous species or oviparous blastodermal cell of a first oviparous species is not contacted with ovotransferrin comprising a naturally occurring ovotransferrin sequence found in a second oviparous species. In some embodiments of any of the aspects, an oviparous stem cell of a first oviparous species or oviparous blastodermal cell of a first oviparous species is not contacted with mammalian transferrin. In some embodiments of any of the aspects, an oviparous stem cell of a first oviparous species or oviparous blastodermal cell of a first oviparous species is not contacted with transferrin comprising a naturally occurring transferrin sequence found in a mammalian species.

In some embodiments of any of the aspects, an oviparous stem cell of a first avian species or oviparous blastodermal cell of a first avian species is contacted with ovotransferrin of an avian species (e.g., any avian species). In some embodiments of any of the aspects, an oviparous stem cell of a first avian species or oviparous blastodermal cell of a first avian species is contacted with ovotransferrin comprising a naturally occurring ovotransferrin sequence found in an avian species (e.g., any avian species).

In some embodiments of any of the aspects, an oviparous stem cell of a first fish species or oviparous blastodermal cell of a first fish species is contacted with ovotransferrin of a fish species (e.g., any fish species). In some embodiments of any of the aspects, an oviparous stem cell of a first fish species or oviparous blastodermal cell of a first fish species is contacted with ovotransferrin comprising a naturally occurring ovotransferrin sequence found in a fish species (e.g., any fish species).

As used herein “ovotransferrin” refers to a member of the transferrin and metalloproteinase glycoprotein found in egg white albumen. The structure and function of ovotransferrin is known in the art, e.g., Acero-Lopez Food Research International 2012 46:480-487; which is incorporated by reference herein in its entirety. Sequences of ovotransferrin for a number of species are known in the art, e.g., chicken ovotransferrin (NBCI Gene ID: 396241), ostrich (NCBI Gene ID: 104138946), duck (NCBI Gene ID: 101795303), quail (NCBI Gene ID: 107317970), and turkey (NCBI Gene ID: 100125333). In some embodiments of any of the aspects, the ovotransferrin is ovotransferrin polypeptide. In some embodiments of any of the aspects, the ovotransferrin comprises the N and C terminal lobes of ovotransferrin.

In some embodiments of any of the aspects, the ovotrasferrin is an ovotransferrin from an oviparous species. In some embodiments of any of the aspects, the ovotransferrin is an avian ovotransferrin. In some embodiments of any of the aspects, the ovotransferrin is an Gallus gallus ovotransferrin. In some embodiments of any of the aspects, the ovotransferrin is an Gallus gallus domesticus ovotransferrin. In some embodiments of any of the aspects, the ovotransferrin comprises chicken ovotransferrin (e.g., SEQ ID NO: 1). In some embodiments of any of the aspects, the ovotransferrin consists of chicken ovotransferrin (e.g., SEQ ID NO: 1).

In some embodiments of any of the aspects, the ovotransferrin comprises a sequence with at least 70% sequence homology to SEQ ID NO: 1. In some embodiments of any of the aspects, the ovotransferrin comprises a sequence with at least 75% sequence homology to SEQ ID NO: 1. In some embodiments of any of the aspects, the ovotransferrin comprises a sequence with at least 80% sequence homology to SEQ ID NO: 1. In some embodiments of any of the aspects, the ovotransferrin comprises a sequence with at least 85% sequence homology to SEQ ID NO: 1. In some embodiments of any of the aspects, the ovotransferrin comprises a sequence with at least 90% sequence homology to SEQ ID NO: 1. In some embodiments of any of the aspects, the ovotransferrin comprises a sequence with at least 95% sequence homology to SEQ ID NO: 1. In some embodiments of any of the aspects, the ovotransferrin retains the wild-type metal-binding activity of a natural-occurring reference ovotransferrin.

In some embodiments of any of the aspects, the ovotransferrin comprises a sequence with at least 80% sequence identity to SEQ ID NO: 1. In some embodiments of any of the aspects, the ovotransferrin comprises a sequence with at least 85% sequence identity to SEQ ID NO: 1. In some embodiments of any of the aspects, the ovotransferrin comprises a sequence with at least 90% sequence identity to SEQ ID NO: 1. In some embodiments of any of the aspects, the ovotransferrin comprises a sequence with at least 95% sequence identity to SEQ ID NO: 1. In some embodiments of any of the aspects, the ovotransferrin retains the wild-type metal-binding activity of a natural-occurring reference ovotransferrin.

In some embodiments of any of the aspects, the ovotransferrin comprises an ovotransferrin from the same species as the at least one avian stem cell or avian blastodermal cell.

(chicken ovotransferrin, mature polypeptide)
SEQ ID NO: 1
a ppksvirwct isspeekkcn nlrdltqqer isltcvqkat
yldcikaian neadaisldg gqvfeaglap yklkpiaaev yehtegstts yyavavvkkg
teftvndlqg ktschtglgr sagwnipigt lihrgaiewe giesgsvega vakffsascv
pgatieqklc rqckgdpktk carnapysgy sgafhclkdg kgdvafvkht tvnenapdqk
deyellcldg srqpvdnykt cnwarvaaha vvarddskve diwsflskaq sdfgvdtksd
fhlfgppgkk dpvlkdllfk dsaimlkrvp slmdsqlylg feyysaiqsm rkdqltpspr
enriqwcavg kdekskcdrw svvsngdvec tvvdetkdci ikimkgeada valdgglvyt
agvcglvpvm aeryddesqc sktderpasy favavarkds nvnwnnlkgk kschtavgrt
agwvipmgli hnrtgtonfd eyfsegcapg sppnsrlcql cqgsggippe rcvassheky
fgytgalrcl vekgdvafvq hstveentgg knkadwaknl qmddfellct dgrranvmdy
recnlaevpt havvvrpeka nkirdllerq ekrfgvngse kskfmmfesq nkdllfkdlt
kclfkvregt tykeflgdkf ytvisslktc npsdilqmcs flegk

Ovotransferrin sequences for other oviparous species are known in the art. By way of non-limiting example, sequences for the ovotransferrins of the following species are readily available in the NCBI database. Where reference is made herein to NCBI ID numbers, the sequence data available as of Feb. 14, 2023 is referred to.

| | |
|---|---|
| Chicken | NP_990635.2 |
| Emu | Genbank BAJ23165.1 |
| Ruddy duck | XP_035190671.1 |
| Tufted duck | XP_032049143 |
| Mallard | P56410.1 |
| Ring-necked pheasant | XP_031452059 |

In embodiments relating to fish, ovotranferrin is known in the art as “serotransferrin.” Serotransferrin sequences for fishes are known in the art. By way of non-limiting example, sequences for the transferrins of the following species are readily available in the Uniprot database. Where reference is made herein to Uniprot ID numbers, the sequence data available as of Feb. 14, 2023 is referred to.
Danio rerio (Zebrafish): A8WGJ5
Acanthochromis polyacanthus (spiny chromis): A0A3Q1F6T2
Acipenser gueldenstaedtii (Russian sturgeon): A0A7S6IKM2
Acipenser ruthenus (Sterlet sturgeon): A0A444U8B4
Ammodytes marinus (lesser sand-eel): Q9DDI9
Anabas testudineus (Climbing perch): A0A7N6A7T7
Carassius auratus (Goldfish): I6QMX9
Carassius cuvieri (Japanese white crucian carp): Q7T1G7
Labrus bergylta (ballan wrasse): A0A3Q3G7M3
Scophthalmus maximus (Turbot): A0A2U9BK56

Ovotransferrin is found in egg white. Accordingly, contacting a cell with ovotransferrin can comprise contacting a cell with an egg extract comprising ovotransferrin. As used herein, “egg extract” refers to a composition comprising any portion or fraction of the material found inside the shell of an egg. In some embodiments of any of the aspects, an egg extract comprises egg yolk. In some embodiments of any of the aspects, an egg extract comprises egg white. In some embodiments of any of the aspects, an egg extract consists of egg white. In some embodiments of any of the aspects, contacting a cell with ovotransferrin can comprise contacting a cell with an egg white extract comprising ovotransferrin.

In some embodiments of any of the aspects, contacting acell with a protein described herein, e.g, ovotransferrin, comprises adding the protein to a cell culture medium, or contacting the cell with a cell culture medium comprising the protein.

In some embodiments of any of the aspects, contacting a cell with a protein described herein, e.g, ovotransferrin, comprises ectopically expressing the protein in the cell. Ectopic expression can be accomplished with a vector, e.g, an integrating or non-integrating vector, using a constitute or inducible promoter. In some embodiments of any of the aspects, contacting the oviparous stem cell or oviparous blastodermal cell with ovotransferrin comprises ectopically expressing the ovotransferrin in the oviparous stem cell or oviparous blastodermal cell

In some embodiments of any of the aspects, contacting a cell with a protein described herein, e.g, ovotransferrin, comprises expressing the protein in a feeder cell e.g., via paracrine signaling. Expression can be endogenous expression, or accomplished with a vector, e.g, an integrating or non-integrating vector, using a constitute or inducible promoter. In some embodiments of any of the aspects, contacting the oviparous stem cell or oviparous blastodermal cell with ovotransferrin comprises culturing the cell with a feeder cell expressing the ovotransferrin. In some embodiments of any of the aspects, contacting the oviparous stem cell or oviparous blastodermal cell with ovotransferrin comprises culturing the cell with a feeder cell ectopically expressing the ovotransferrin.

In some embodiments of any of the aspects, feeder cells can be fibroblasts, embryonic fibroblasts, or derived from/engineered from fibroblasts or embryonic fibroblasts. In some embodiments of any of the aspects, the feeder cells can be of an of oviparous species. In some embodiments of any of the aspects, the feeder cells can be of the same species as the oviparous stem cell or oviparous blastodermal cell.

As demonstrated in the Examples herein, further contacting the at least one oviparous stem cell or oviparous blastodermal cell with an activin receptor-like kinase 4, -5, and/or -7 inhibitor decreases the rate and/or incidence of differentiation. Accordingly, in some embodiments of any of the aspects, the at least one oviparous stem cell or oviparous blastodermal cell is further contacted with an activin receptor-like kinase 4, -5, and -7 inhibitor. In some embodiments of any of the aspects, the at least one oviparous stem cell or oviparous blastodermal cell is further contacted with an activin receptor-like kinase 4, -5, and -7 inhibitor while being contacted with at least one of a Wnt inhibitor; a protein kinase C (PKC inhibitor); and ovotransferrin.

The activin receptor-like kinases ALK4, ALK5 (also known as TGFBR1) and ALK7 transduce TGF-beta superfamily growth and differentiation factors. The ligands, signaling partners, and downstream effects of ALKs are known in the art.

Inhibitors that are selective for one or more ALKs are known in the art. In some embodiments of any of the aspects, the activin receptor-like kinase 4, -5, and/or -7 inhibitor inhibits ALK4. In some embodiments of any of the aspects, the activin receptor-like kinase 4, -5, and/or -7 inhibitor inhibits ALK5. In some embodiments of any of the aspects, the activin receptor-like kinase 4, -5, and/or -7 inhibitor inhibits ALK7. In some embodiments of any of the aspects, the activin receptor-like kinase 4, -5, and/or -7 inhibitor inhibits ALK4 and ALK5. In some embodiments of any of the aspects, the activin receptor-like kinase 4, -5, and/or -7 inhibitor inhibits ALK4 and ALK7. In some embodiments of any of the aspects, the activin receptor-like kinase 4, -5, and/or -7 inhibitor inhibits ALK7 and ALK5. In some embodiments of any of the aspects, the activin receptor-like kinase 4, -5, and/or -7 inhibitor inhibits ALK4, ALK5, and ALK7. In some embodiments of any of the aspects, the activin receptor-like kinase 4, -5, and/or -7 inhibitor does not inhibit ALKs other than ALK4, ALK5, and ALK7.

Exemplary, but non-limiting activin receptor-like kinase 4, -5, and -7 inhibitors include SB431452, A 83-01, SB505124, and AZ12601011. In some embodiments of any of the aspects, the activin receptor-like kinase 4, -5, and -7 inhibitor comprises SB431542 (4-[4-(2H-1,3-Benzodioxol-5-yl)-5-(pyridin-2-yl)-1H-imidazol-2-yl]benzamide). In some embodiments of any of the aspects, the activin receptor-like kinase 4, -5, and -7 inhibitor consists of SB431542 (4-[4-(2H-1,3-Benzodioxol-5-yl)-5-(pyridin-2-yl)-1H-imidazol-2-yl]benzamide). In some embodiments of any of the aspects, the activin receptor-like kinase 4, -5, and -7 inhibitor is SB431542 (4-[4-(2H-1,3-Benzodioxol-5-yl)-5-(pyridin-2-yl)-1H-imidazol-2-yl]benzamide).

As demonstrated in the Examples herein, contacting the at least one oviparous stem cell or oviparous blastodermal cell with a LIF polypeptide promotes the self-renewal of at least one oviparous stem cell or oviparous blastodermal cell. Accordingly in some embodiments of any of the aspects, the at least one oviparous stem cell or oviparous blastodermal cell is further contacted with a LIF polypeptide. In some embodiments of any of the aspects, the at least one oviparous stem cell or oviparous blastodermal cell is further contacted with the LIF polypeptide while being contacted with at least one of a Wnt inhibitor; a protein kinase C (PKC inhibitor); and ovotransferrin. In some embodiments of any of the aspects, the at least one oviparous stem cell or oviparous blastodermal cell is further contacted with a LIF polypeptide while being contacted with the activin receptor-like kinase 4, -5, and -7 inhibitor and at least one of a Wnt inhibitor; a protein kinase C (PKC inhibitor); and ovotransferrin.

As used herein “LIF” or “leukemia inhibitor factor” refers to a cytokine bound by the LIF receptor that activated JAK/STAT and MAPK signaling cascades. The structure and function of LIF is known in the art, e.g., Hinds et al. JBC 1998 273:13738-45; which is incorporated by reference herein in its entirety. Sequences of LIF for a number of species are known in the art, e.g., chicken LIF (NBCI Gene ID: 427718), ostrich (NCBI Gene ID: 104141136), duck (NCBI Gene ID: 110353607), quail (NCBI Gene ID: 107321525), and turkey (NCBI Gene ID: 104913573). In some embodiments of any of the aspects, the LIF is LIF polypeptide.

In some embodiments of any of the aspects, the LIF is an oviparous LIF. In some embodiments of any of the aspects, the LIF is an avian LIF. In some embodiments of any of the aspects, the LIF is a Gallus gallus LIF. In some embodiments of any of the aspects, the LIF is a Gallus gallus domesticus LIF. In some embodiments of any of the aspects, the LIF comprises chicken LIF (e.g., SEQ ID NO: 2). In some embodiments of any of the aspects, the LIF consists of chicken LIF (e.g. SEQ ID NO: 2).

In some embodiments of any of the aspects, the LIF comprises a sequence with at least 80% homology to SEQ ID NO: 2. In some embodiments of any of the aspects, the LIF comprises a sequence with at least 85% sequence homology to SEQ ID NO: 2. In some embodiments of any of the aspects, the LIF comprises a sequence with at least 90% sequence homology to SEQ ID NO: 2. In some embodiments of any of the aspects, the LIF comprises a sequence with at least 95% sequence homology to SEQ ID NO: 2. In some embodiments of any of the aspects, the LIF retains the wild-type receptor-binding and/or receptor-activating activity of a natural-occurring reference LIF.

In some embodiments of any of the aspects, the LIF comprises a sequence with at least 80% sequence identity to SEQ ID NO: 2. In some embodiments of any of the aspects, the LIF comprises a sequence with at least 85% sequence identity to SEQ ID NO: 2. In some embodiments of any of the aspects, the LIF comprises a sequence with at least 90% sequence identity to SEQ ID NO: 2. In some embodiments of any of the aspects, the LIF comprises a sequence with at least 95% sequence identity to SEQ ID NO: 2. In some embodiments of any of the aspects, the LIF retains the wild-type receptor-binding and/or receptor-activating activity of a natural-occurring reference LIF.

It is contemplated herein that maintenance of stem cells, e.g., expanding or culturing stem cells in vitro requires a LIF that is appropriately matched to the origin of the stem cell. That is, mammalian LIF cannot optimally support maintenance, expansion, or culture of oviparous stem cells—rather, an oviparous LIF is required. Species-matched LIF can provide even more effective results, e.g., when the cell and the LIF originate from the same species. Accordingly, in some embodiments of any of the aspects, an oviparous stem cell of a first oviparous species or oviparous blastodermal cell of a first oviparous species is contacted with LIF of the first oviparous species. In some embodiments of any of the aspects, an oviparous stem cell of a first oviparous species or oviparous blastodermal cell of a first oviparous species is contacted with LIF comprising a naturally occurring LIF sequence found in the first oviparous species.

In some embodiments of any of the aspects, the LIF has a sequence with at least 70% sequence homology to an endogenous LIF encoded in a genome of the at least one oviparous stem cell or oviparous blastodermal cell. In some embodiments of any of the aspects, the LIF has a sequence with at least 80% sequence homology to an endogenous LIF encoded in a genome of the at least one oviparous stem cell or oviparous blastodermal cell. In some embodiments of any of the aspects, the LIF has a sequence with at least 85% sequence homology to an endogenous LIF encoded in a genome of the at least one oviparous stem cell or oviparous blastodermal cell. In some embodiments of any of the aspects, the LIF has a sequence with at least 90% sequence homology to an endogenous LIF encoded in a genome of the at least one oviparous stem cell or oviparous blastodermal cell. In some embodiments of any of the aspects, the LIF has a sequence with at least 95% sequence homology to an endogenous LIF encoded in a genome of the at least one oviparous stem cell or oviparous blastodermal cell.

In some embodiments of any of the aspects, the LIF has a sequence with at least 80% sequence identity to an endogenous LIF encoded in a genome of the at least one oviparous stem cell or oviparous blastodermal cell. In some embodiments of any of the aspects, the LIF has a sequence with at least 85% sequence identity to an endogenous LIF encoded in a genome of the at least one oviparous stem cell or oviparous blastodermal cell. In some embodiments of any of the aspects, the LIF has a sequence with at least 90% sequence identity to an endogenous LIF encoded in a genome of the at least one oviparous stem cell or oviparous blastodermal cell. In some embodiments of any of the aspects, the LIF has a sequence with at least 95% sequence identity to an endogenous LIF encoded in a genome of the at least one oviparous stem cell or oviparous blastodermal cell. In some embodiments of any of the aspects, the LIF has a sequence identical to an endogenous LIF encoded in a genome of the at least one oviparous stem cell or oviparous blastodermal cell.

In some embodiments of any of the aspects, an oviparous stem cell of a first oviparous species or oviparous blastodermal cell of a first oviparous species is not contacted with LIF comprising a naturally occurring LIF sequence found in a second oviparous species. In some embodiments of any of the aspects, an oviparous stem cell of a first oviparous species or oviparous blastodermal cell of a first oviparous species is not contacted with mammalian LIF. In some embodiments of any of the aspects, an oviparous stem cell of a first oviparous species or oviparous blastodermal cell of a first oviparous species is not contacted with LIF comprising a naturally occurring LIF sequence found in a mammalian species.

In some embodiments of any of the aspects, an oviparous stem cell of a first avian species or oviparous blastodermal cell of a first avian species is contacted with LIF of an avian species (e.g, any avian species). In some embodiments of any of the aspects, an oviparous stem cell of a first avian species or oviparous blastodermal cell of a first avian species is contacted with LIF comprising a naturally occurring LIF sequence found in an avian species (e.g., any avian species).

In some embodiments of any of the aspects, an oviparous stem cell of a first fish species or oviparous blastodermal cell of a first fish species is contacted with LIF of a fish species (e.g., any fish species). In some embodiments of any of the aspects, an oviparous stem cell of a first fish species or oviparous blastodermal cell of a first fish species is contacted with LIF comprising a naturally occurring LIF sequence found in a fish species (e.g., any fish species).

In some embodiments of any of the aspects, the LIF comprises an LIF from the same species as the at least one oviparous stem cell or at least one oviparous blastodermal cell.

| (chicken LIF) | |
|---|---|
| SEQ ID NO: 2 | |
| 1 | mrlipagvvp fvallllqrr pvsgrallgt ssacptnglc ranvleqtrr qvallnataq |
| 61 | dlfslylkcq gepfssesdr lcspsgiffp pfhvnrtter kevmvamykl faflnaslgn |
| 121 | itrdqeelnp makellnrlh nttkttrgli snltcllckh ynifqvdvsy gesskdksaf |
| 181 | kkkqqgcqvl rkyvqviaqa arvllphlsp a |

In some embodiments of any of the aspects, contacting a cell with a protein described herein, e.g, LIF, comprises adding the protein to a cell culture medium, or contacting the cell with a cell culture medium comprising the protein.

In some embodiments of any of the aspects, contacting a cell with a protein described herein, e.g, LIF, comprises ectopically expressing the protein in the cell. Ectopic expression can be accomplished with a vector, e.g, an integrating or non-integrating vector, using a constitute or inducible promoter. In some embodiments of any of the aspects, contacting the oviparous stem cell or oviparous blastodermal cell with LIF comprises ectopically expressing the LIF in the oviparous stem cell or oviparous blastodermal cell

In some embodiments of any of the aspects, contacting a cell with a protein described herein, e.g, LIF, comprises expressing the protein in a feeder cell e.g., via paracrine signaling. Expression can be endogenous expression, or accomplished with a vector, e.g, an integrating or non-integrating vector, using a constitute or inducible promoter. In some embodiments of any of the aspects, contacting the oviparous stem cell or oviparous blastodermal cell with LIF comprises culturing the cell with a feeder cell expressing the LIF. In some embodiments of any of the aspects, contacting the oviparous stem cell or oviparous blastodermal cell with LIF comprises culturing the cell with a feeder cell ectopically expressing the LIF.

Ovotransferrin is a component of the egg white. Thus, in some embodiments of any of the aspects, the at least one oviparous stem cell or at least one oviparous blastodermal cell is contacted with egg white. Furthermore, in some embodiments of any of the aspects, the at least one oviparous stem cell or at least one oviparous blastodermal cell is contacted by the liquid fraction of the whole egg. In some embodiments of any of the aspects, the liquid fraction of the entire egg is of avian origin. In some embodiments of any of the aspects, the liquid fraction from the entire egg is of non-avian origin.

In some embodiments of any of the aspects, contacting the at least one oviparous stem cell or at least one oviparous blastodermal cell with ovotransferrin can comprise contacting the at least one oviparous stem cell or at least one oviparous blastodermal cell with egg white. In some embodiments of any of the aspects, contacting the at least one oviparous stem cell or at least one oviparous blastodermal cell with ovotransferrin can comprise contacting the at least one oviparous stem cell or at least one oviparous blastodermal cell with the liquid fraction of a whole egg.

As described in the Examples herein, inclusion of egg yolk in the growth media improves the growth of the at least one oviparous stem cell or at least one oviparous blastodermal cell. Accordingly, in some embodiments of any of the aspects, the at least one oviparous stem cell or at least one oviparous blastodermal cell is further contacted with egg yolk. In some embodiments of any of the aspects, the at least one oviparous stem cell or at least one oviparous blastodermal cell is further contacted with egg yolk while being contacted with at least one of a Wnt inhibitor; a protein kinase C (PKC inhibitor); and ovotransferrin. In some embodiments of any of the aspects, the at least one oviparous stem cell or at least one oviparous blastodermal cell is further contacted with egg yolk while being contacted with LIF and at least one of a Wnt inhibitor; a protein kinase C (PKC inhibitor); and ovotransferrin; and LIF. In some embodiments of any of the aspects, the at least one oviparous stem cell or at least one oviparous blastodermal cell is further contacted with egg yolk while being contacted with the activin receptor-like kinase 4, -5, and -7 inhibitor and at least one of a Wnt inhibitor; a protein kinase C (PKC inhibitor); and ovotransferrin. In some embodiments of any of the aspects, the at least one oviparous stem cell or at least one oviparous blastodermal cell is further contacted with egg yolk while being contacted with LIF, the activin receptor-like kinase 4, -5, and -7 inhibitor, and at least one of a Wnt inhibitor; a protein kinase C (PKC inhibitor); and ovotransferrin.

It is contemplated herein that maintenance of stem cells, e.g., expanding or culturing stem cells in vitro is promoted by egg yolk that is appropriately matched to the origin of the stem cell. Species-matched egg yolk can provide even more effective results, e.g., when the cell and the egg yolk originate from the same species. Accordingly, in some embodiments of any of the aspects, an oviparous stem cell of a first oviparous species or oviparous blastodermal cell of a first oviparous species is contacted with egg yolk of the first oviparous species. In some embodiments of any of the aspects, an oviparous stem cell of a first oviparous species or oviparous blastodermal cell of a first oviparous species is not contacted with egg yolk of a second oviparous species.

In some embodiments of any of the aspects, an oviparous stem cell of a first avian species or oviparous blastodermal cell of a first avian species is contacted with egg yolk of an avian species (e.g, any avian species). In some embodiments of any of the aspects, the egg yolk is avian egg yolk.

In some embodiments of any of the aspects, an oviparous stem cell of a first fish species or oviparous blastodermal cell of a first fish species is contacted with egg yolk of a fish species (e.g., any fish species).

In some embodiments of any of the aspects, the egg yolk is whole egg yolk of an oviparous species. In some embodiments of any of the aspects, the egg yolk comprises a 50-100 kDa fraction of avian egg yolk. In some embodiments of any of the aspects, the egg yolk consists of a 50-100 kDa fraction of avian egg yolk. In some embodiments of any of the aspects, the avian egg yolk comprises egg yolk from the same species as the at least one oviparous stem cell or oviparous blastodermal cell. In some embodiments of any of the aspects, the egg yolk consists of egg yolk from the same species as the at least one oviparous stem cell or oviparous blastodermal cell.

In some embodiments of any of the aspects, the at least one oviparous stem cell or at least one oviparous blastodermal cell can be a primer cell. In some embodiments of any of the aspects, the at least one avian stem cell can be a primary cell. In some embodiments of any of the aspects, the at least one oviparous blastodermal cell can be a primary cell. In some embodiments of any of the aspects, the at least one oviparous blastodermal cell can be a primary cell isolated from an oviparous embryo. In some embodiments of any of the aspects, the method can further comprise a step of isolating a blastodermal cell from an oviparous embryo prior to contacting the at least one oviparous blastodermal cell with at least one of a Wnt inhibitor; a protein kinase C (PKC inhibitor); and ovotransferrin.

Avian embryo development is described in the art according to EGK stages (see, e.g., Eyal-Giladi and Kochav. Dev Biol 1976 49:321-337, which is incorporated by reference herein in its entirety). In some embodiments of any of the aspects, the avian embryo is a EGK.X to EGK.XII stage embryo. In some embodiments of any of the aspects, the avian embryo is a EGK.X stage embryo. In some embodiments of any of the aspects, the avian embryo is a EGK.XI stage embryo. In some embodiments of any of the aspects, the avian embryo is a EGK.XII stage embryo.

As demonstrated herein, the methods described herein are applicable and effective for a number of oviparous species. Oviparous species, as incorporated by reference herein, include any animal species whose native embryonic development occurs in ovo, i.e. within the shell of an extracorporeal egg. Oviparous species include those designated as avian as well as certain species designated as amphibian, reptilian and fish species. The embodiments described herein refer to oviparous species.

In some embodiments of any of the aspects, the oviparous stem cells, at least one oviparous stem cell, and/or at least one oviparous blastodermal cell is of an avian species or avian. In some embodiments of any of the aspects, the avian species or avian is Gallus gallus, Meleagris gallopavo, Gallus gallus domesticus, a Phasiandiae; Phasianus colchicus; Coturnix coturnix; a Galliformes; Anas platyrhynchos, Anser anser, (including duck and goose); an Anseriformes; an Anatinae; Meleagris gallopavo domesticus; or Struthio camelus, a Struthioformes; or a peafowl (e.g, Indian peafowl (Pavo cristatus), green peafowl (Pavo muticus), or Congo peafowl (Afropavo congensis). In some embodiments of any of the aspects, the avian is Gallus gallus. In some embodiments of any of the aspects, the avian is Meleagris Gallopavo. In some embodiments of any of the aspects, the avian is Gallus gallus domesticus. In some embodiments of any of the aspects, the avian is a Phasiandiae. In some embodiments of any of the aspects, the avian is Phasianus colchicus. In some embodiments of any of the aspects, the avian is Coturnix coturnix. In some embodiments of any of the aspects, the avian is Anas platyrhynchos, In some embodiments of any of the aspects, the avian is Anser anser, In some embodiments of any of the aspects, the avian is an Anseriformes. In some embodiments of any of the aspects, the avian is an Anatinae. In some embodiments of any of the aspects, the avian is Meleagris gallopavo. In some embodiments of any of the aspects, the avian is Meleagris gallopavo domesticus. In some embodiments of any of the aspects, the avian is Struthio camelus.

In somembodiments of any of the aspects, the species is non-avian and oviparous.

As described in the examples herein, chicken, quail, goose, pheasant, duck and/or turkey cells display high growth, self-renewal, and pluripotency when cultured or maintained by methods utilizing at least one Wnt inhibitor, at least one PKC inhibitor, at least one activin receptor-like kinase 4, -5, and/or -7 inhibitor, and LIF. Accordingly, in embodiments where the at least one avian stem cell or avian blastodermal cell is a chicken, quail, goose, pheasant, duck and/or turkey cell, the method can comprise contacting with at least one Wnt inhibitor, at least one PKC inhibitor, at least one activin receptor-like kinase 4, -5, and/or -7 inhibitor, and LIF; or the composition or combination can comprise at least one Wnt inhibitor, at least one PKC inhibitor, at least one activin receptor-like kinase 4, -5, and/or -7 inhibitor, and LIF.

As described in the examples herein, ostrich cells display high growth, self-renewal, and pluripotency when cultured or maintained by methods utilizing at least one Wnt inhibitor, at least one PKC inhibitor, at least one activin receptor-like kinase 4, -5, and/or -7 inhibitor, and ovotransferrin. Accordingly, in embodiments where the at least one avian stem cell or avian blastodermal cell is an ostrich cell, the method can comprise contacting with at least one Wnt inhibitor, at least one PKC inhibitor, at least one activin receptor-like kinase 4, -5, and/or -7 inhibitor, and ovotransferrin; or the composition or combination can comprise at least one Wnt inhibitor, at least one PKC inhibitor, at least one activin receptor-like kinase 4, -5, and/or -7 inhibitor, and ovotransferrin.

In some embodiments of any of the aspects, the contacting step occurs in a cell culture. In some embodiments of any of the aspects, the contacting step comprises contacting the at least one oviparous stem cell or at least one oviparous blastodermal cell with a cell culture medium comprising at least one of a Wnt inhibitor, a protein kinase C (PKC inhibitor), and ovotransferrin; and optionally one or more of LIF, egg extract, egg yolk, and activin receptor-like kinase 4, -5, and -7 inhibitor. In some embodiments of any of the aspects, the contacting step comprises contacting the at least one oviparous stem cell or at least one oviparous blastodermal cell with a cell culture medium comprising at least one of a Wnt inhibitor, a protein kinase C (PKC inhibitor), and egg extract; and optionally one or more of LIF, egg yolk, and activin receptor-like kinase 4, -5, and -7 inhibitor.

In some embodiments of any of the aspects, the cell culture medium is a serum-free medium. In some embodiments of any of the aspects, the cell culture medium is a basal medium including a solution of biological molecules such as fatty acids, lipids, amino acids, vitamins minerals, sugars and other relevant molecules (e.g. Dulbecco's Modified Eagle Medium/Nutrient Mixture F-12 (DMEM/F12)). In some embodiments of any of the aspects, the cell culture medium is a serum-free basal medium.

In some embodiments of any of the aspects, the cell culture medium is a serum-free medium comprising Dulbecco's Modified Eagle Medium/Nutrient Mixture F-12 (DMEM/F12), Neuralbasal, N2, and B27.

In some embodiments of any of the aspects, the contacting step is conducted for at least one passage. In some embodiments of any of the aspects, the contacting step is conducted for at least two passages.

In some embodiments of any of the aspects, the contacting step is conducted for at least three passages. In some embodiments of any of the aspects, the contacting step is conducted for at least four passages.

In some embodiments of any of the aspects, the contacting step is conducted for at least 5 days. In some embodiments of any of the aspects, the contacting step is conducted for at least 10 days. In some embodiments of any of the aspects, the contacting step is conducted for at least 12 days. In some embodiments of any of the aspects, the contacting step is conducted for at least 2 weeks. In some embodiments of any of the aspects, the contacting step is conducted for at least 3 weeks. In some embodiments of any of the aspects, the contacting step is conducted for at least 4 weeks.

In some embodiments of any of the aspects, the contacting step comprises adding at least one of a Wnt inhibitor, a protein kinase C (PKC inhibitor), and ovotransferrin; and optionally one or more of LIF, egg extract, egg yolk, and activin receptor-like kinase 4, -5, and -7 inhibitor to a cell culture medium repeatedly. In some embodiments of any of the aspects, the contacting step comprises adding at least one of a Wnt inhibitor, a protein kinase C (PKC inhibitor), and ovotransferrin; and optionally one or more of LIF, egg extract, egg yolk, and activin receptor-like kinase 4, -5, and -7 inhibitor to a cell culture medium frequently enough that the cell culture medium continuously comprises a detectable level (or other level specified herein) of the at least one of a Wnt inhibitor, a protein kinase C (PKC inhibitor), and ovotransferrin; and optionally one or more of LIF, egg extract egg yolk, and activin receptor-like kinase 4, -5, and -7 inhibitor.

In some embodiments of any of the aspects, the contacting step comprises culturing the at least one oviparous stem cell or at least one oviparous blastodermal cell with repeated medium changes. In some embodiments of any of the aspects, the contacting step comprises culturing the at least one oviparous stem cell or at least one oviparous blastodermal cell with daily medium changes. In some embodiments of any of the aspects, the medium change comprises providing fresh cell culture medium comprising the at least one of a Wnt inhibitor, a protein kinase C (PKC inhibitor), and ovotransferrin; and optionally one or more of LIF, egg extract, egg yolk, and activin receptor-like kinase 4, -5, and -7 inhibitor.

In some embodiments of any of the aspects, the contacting step comprises culturing the at least one oviparous stem cell or at least one oviparous blastodermal cell at 38° C. in 5% CO₂ with daily medium changes. In some embodiments of any of the aspects, the contacting step comprises culturing the at least one oviparous stem cell or at least one oviparous blastodermal cell at 38° C. in 5% CO₂.

In some embodiments of any of the aspects, the methods described herein produce, maintain, or expand germline competent and pluripotent stem cells. In some embodiments of any of the aspects, the methods described herein produce, maintain, or expand germline competent stem cells. In some embodiments of any of the aspects, the methods described herein produce, maintain, or expand pluripotent stem cells. In some embodiments of any of the aspects, the methods described herein produce, maintain, or expand morphologically undifferentiated stem cells. In some embodiments of any of the aspects, the methods described herein produce, maintain, or expand cells expressing Nanog mRNA. Pluripotent and lineage-restricted cells can be differentiated in some embodiments by the amount of cytosine which is methylated, with pluripotent cells having lower percentages of methylated cytosine. Accordingly, in some embodiments of any of the aspects, the methods described herein produce, maintain, or expand cells having less than 5%, less than 4%, or less than 3% methyl-cytosine (as a percentage of total cytosine content).

In some embodiments of any of the aspects, the ovotransferrin is at a concentration of from about 1 μg/ml to about 300 μg/ml. In some embodiments of any of the aspects, the ovotransferrin is at a concentration of about 20 μg/ml.

In some embodiments of any of the aspects,
a) the Wnt inhibitor is at a concentration of about 50 nM to about 30 μM;
b) the PKC inhibitor is at a concentration of about 100 nM to about 40 μM;
c) the activin receptor like kinase 4, -5, and -7 inhibitor is at a concentration of about 50 nM to about 35 μM; and/or
d) the LIF polypeptide is at a concentration of about 0.5 ng/ml to about 500 ng/ml.

In some embodiments of any of the aspects, the ovotransferrin is at a concentration of from about 5 μg/ml to about 60 μg/ml. In some embodiments of any of the aspects, the ovotransferrin is at a concentration of about 20 μg/ml.

In some embodiments of any of the aspects,
a) the Wnt inhibitor is at a concentration of about 250 nM to about 6 μM;
b) the PKC inhibitor is at a concentration of about 500 nM to about 8 μM;
c) the activin receptor like kinase 4, -5, and -7 inhibitor is at a concentration of about 0.25 μM to about 7 μM; and/or
d) the LIF polypeptide is at a concentration of about 2.5 ng/ml to about 100 ng/ml.

In some embodiments of any of the aspects, the ovotransferrin is at a concentration of from about 10 μg/ml to about 30 μg/ml. In some embodiments of any of the aspects, the ovotransferrin is at a concentration of about 20 μg/ml.

In some embodiments of any of the aspects,
a) the Wnt inhibitor is at a concentration of about 0.5 μM to about 3p M;
b) the PKC inhibitor is at a concentration of about 1 μM to about 4 μM;
c) the activin receptor like kinase 4, -5, and -7 inhibitor is at a concentration of about 0.5 μM to about 3.5p M; and/or
d) the LIF polypeptide is at a concentration of about 5 ng/ml to about 50 ng/ml.

In some embodiments of any of the aspects,
a) the Wnt inhibitor is at a concentration of about 2.5 μM;
b) the PKC inhibitor is at a concentration of about 2 μM;
c) the activin receptor like kinase 4, -5, and -7 inhibitor is at a concentration of about 2.5 μM; and
d) the LIF polypeptide is at a concentration of about 10 ng/ml or about 40 ng/ml.

In one aspect of any of the embodiments, described herein is a composition or combination comprising: at least one Wnt inhibitor; at least one protein kinase C (PKC) inhibitor; and ovotransferrin. In one aspect of any of the embodiments, described herein is a composition or combination comprising: at least one Wnt inhibitor and at least one protein kinase C (PKC) inhibitor.

In one aspect of any of the embodiments, described herein is a composition or combination comprising: at least one Wnt inhibitor and ovotransferrin. In one aspect of any of the embodiments, described herein is a composition or combination comprising: at least one protein kinase C (PKC) inhibitor and ovotransferrin.

In some embodiments of any of the aspects, the composition or combination further comprises at least one activin receptor-like kinase 4, -5, and/or -7 inhibitor. In some embodiments of any of the aspects, the composition or combination further comprises a leukemia inhibitory factor (LIF) polypeptide. In some embodiments of any of the aspects, the composition or combination further comprises egg yolk. In some embodiments of any of the aspects, the composition or combination further comprises at least one activin receptor-like kinase 4, -5, and/or -7 inhibitor and a leukemia inhibitory factor (LIF) polypeptide. In some embodiments of any of the aspects, the composition or combination further comprises at least one activin receptor-like kinase 4, -5, and/or -7 inhibitor and egg yolk. In some embodiments of any of the aspects, the composition or combination further comprises a leukemia inhibitory factor (LIF) polypeptide and egg yolk. In some embodiments of any of the aspects, the composition or combination further comprises at least one activin receptor-like kinase 4, -5, and/or -7 inhibitor; a leukemia inhibitory factor (LIF) polypeptide; and egg yolk. In some embodiments of any of the aspects, egg extract is used in place of egg yolk.

In some embodiments of any of the aspects, the composition or combination further comprises cell culture medium. In some embodiments of any of the aspects, the composition or combination further comprises serum-free cell culture medium. In some embodiments of any of the aspects, the composition or combination further comprises basal cell culture medium. In some embodiments of any of the aspects, the composition or combination further comprises serum-free basal cell culture medium. In some embodiments of any of the aspects, the composition or combination further comprises a basal medium including a solution of biological molecules such as fatty acids, lipids, amino acids, vitamins minerals, sugars and other relevant molecules (e.g. medium comprising N2B27 medium comprising Dulbecco's Modified Eagle Medium/Nutrient Mixture F-12 (DMEM/F12):Neuralbasal (1:1) 0.5×N2, and 0.5× B27). In some embodiments of any of the aspects, the composition or combination further comprises cell culture medium comprising N2B27 medium comprising Dulbecco's Modified Eagle Medium/Nutrient Mixture F-12 (DMEM/F12):Neuralbasal (1:1) 0.5×N2, and 0.5×B27.

In some embodiments of any of the aspects, the composition or combination further comprises at least one oviparous stem cell or at least one oviparous blastodermal cell. In some embodiments of any of the aspects, the composition or combination further comprises at least one avian stem cell or at least one avian blastodermal cell. In some embodiments of any of the aspects, the composition or combination further comprises at least one oviparous stem cell. In some embodiments of any of the aspects, the composition or combination further comprises at least one avian stem cell. In some embodiments of any of the aspects, the composition or combination further comprises at least one oviparous blastodermal cell. In some embodiments of any of the aspects, the composition or combination further comprises at least one avian blastodermal cell.

In some embodiments, the elements of a combination are provided in the same formulation. In some embodiments, the elements of a combination are provided in separate formulations or containers. As used herein “combination” refers to a group of two or more substances or elements for use together, e.g., for use in maintain or culturing cells. The two or more substances can be present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the two or more substances can be comprised by the same or different superstructures, e.g., nanoparticles, liposomes, vectors, cells, scaffolds, or the like, and said superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be mixed or brought into contact with each other when cell growth and/or culture is to be performed.

A kit is an assemblage of materials or components, including at least one element or reagent described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to culture cells. Still in accordance with the present invention, “instructions for use” may include a tangible expression describing the preparation of at least one element described herein, such as dilution, mixing, or dosing instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition containing a volume of at least one reagent described herein. The packaging material generally has an external label which indicates the contents and/or purpose of the kit and/or its components.

Disclosed herein are bona fide ESC lines established from oviparous species, e.g., avian species, exemplified herein by chicken ESCs. Chicken ESCs express core pluripotency markers and can efficiently differentiate into cells of all three germ layers. Furthermore, chicken ESCs can form high rates of chimerism when injected into chicken embryos both ex ovo and in ovo. More importantly, chicken ESCs can give rise to germ cells both in vitro and in ovo, indicating that chicken ESCs are germline competent. Interspecies chimeras can also be generated by injecting other avian ESCs into chicken embryos. Establishment of germline competent avian ESCs opens up a new avenue for producing genetically engineered avian species.

According to various embodiments of the present disclosure, a method of deriving embryonic stem cells (ESCs) from avian species includes culturing an embryo extracted from an avian egg in a culture medium to harvest cells from yolk of the avian egg; dissociating cells from the cultured embryo; isolating a morphologically undifferentiated ESC colony from the dissociated cells in a culture medium supplemented with a Wnt inhibitor and a protein kinase C (PKC) inhibitor; and culturing the isolated ESC colony in the presence of ovotransferrin, thereby deriving ESCs. According to various embodiments of the present disclosure, a method of deriving embryonic stem cells (ESCs) from avian species includes culturing an embryo extracted from an avian egg in a culture medium to harvest cells from the germinal disk of the vitelline membrane of the avian egg; dissociating cells from the cultured embryo; isolating a morphologically undifferentiated ESC colony from the dissociated cells in a culture medium supplemented with a Wnt inhibitor and a protein kinase C (PKC) inhibitor; and culturing the isolated ESC colony in the presence of ovotransferrin, thereby deriving ESCs. The derived ESCs are germline competent and pluripotent.

In some embodiments, the Wnt inhibitor includes Wnt/β-catenin signaling inhibitor IWR-1. In some embodiments, the PKC inhibitor includes Gö6983. In some embodiments, the ESCs derived in the presence of the Wnt inhibitor and the PKC inhibitor express Nanog mRNA.

In some embodiments, the method further includes promoting ESC self-renewal by culturing the ESCs in the presence of ovotransferrin, IWR-1, Gö6983, an inhibitor of activin receptor-like kinases-4, -5, and -7, and a leukemia inhibitory factor (LIF). For example, the LIF is chicken LIF. In some embodiments, the inhibitor of activin receptor-like kinases-4, -5, and -7 includes SB431542. In some embodiments, the ovotransferrin is same as ovotransferrin present in the yolk.

In some embodiments, a culture medium used for ESC maintenance includes a serum-free medium comprising Dulbecco's Modified Eagle Medium/Nutrient Mixture F-12 (DMEM/F12), Neuralbasal, N2, B27, and ovotransferrin. In some embodiments, ESCs are continuously passaged beyond 2 passages while remaining morphologically undifferentiated by supplementing with ovotransferrin, IWR-1, Gö6983, SB431542, and chicken LIF. In some embodiments, 100% of the ESCs remain morphologically undifferentiated when supplemented with ovotransferrin, IWR-1, Gö6983, SB431542, and chicken LIF. In some embodiments, at least 90% of the ESCs remain morphologically undifferentiated. In some embodiments, at least 95% of the ESCs remain morphologically undifferentiated. For example, the avian species includes chicken, and derivation and long-term self-renewal of ESCs is supported by ovotransferrin, IWR-1, and Gö6983.

In some embodiments of any of the aspects, the species is oviparous. In some embodiments of any of the aspects, the species is avian. In some embodiments, the avian species includes chicken, quail, goose, or pheasant, and derivation and self-renewal of ESCs is supported at least 2 weeks after culturing the isolated ESC colony by ovotransferrin, IWR-1, Gö6983, SB431542, and chicken LIF. In some embodiments, the avian species further includes duck or turkey. In some embodiments, ESCs are cultured at 38° C. in 5% CO2 with daily medium change.

In some embodiments, the method of deriving ESCs fromoviparous (e.g, avian) species further includes promoting ESC self-renewal by culturing the ESCs in the presence of ovotransferrin, IWR-1, Gö6983, an inhibitor of activin receptor-like kinases-4, -5, and -7. For example, the inhibitor of activin receptor-like kinases-4, -5, and -7 includes SB431542. For example, the ovotransferrin is same as ovotransferrin present in the egg. In some embodiments, a culture medium used for ESC maintenance comprises a serum-free medium comprising Dulbecco's Modified Eagle Medium/Nutrient Mixture F-12 (DMEM/F12), Neuralbasal, N2, B27, and ovotransferrin. In some embodiments, a culture medium used for ESC maintenance comprises a serum-free basal medium including a solution of biological molecules such as fatty acids, lipids, amino acids, vitamins minerals, sugars and other relevant molecules (e.g. Dulbecco's Modified Eagle Medium/Nutrient Mixture F-12 (DMEM/F12), Neuralbasal, N2, B27, and ovotransferrin. In some embodiments, ESCs are continuously passaged beyond 2 passages while remaining morphologically undifferentiated by supplementing with ovotransferrin, IWR-1, Gö6983, and SB431542. In some embodiments, the avian species includes ostrich. In some embodiments, derivation and long-term self-renewal of ESCs is supported by ovotransferrin, IWR-1, and Gö6983.

In some embodiments, the culture medium is used for derivation and/or propagation of ESCs from chicken, quail, goose, pheasant, duck and/or turkey.

According to various embodiments of the present disclosure, a culture medium includes a Wnt inhibitor; a protein kinase C (PKC) inhibitor; ovotransferrin; one or more inhibitors of activin receptor-like kinases-4, -5, and/or -7; and a leukemia inhibitory factor (LIF). In some embodiments, the Wnt inhibitor includes Wnt/β-catenin signaling inhibitor IWR-1. In some embodiments, the PKC inhibitor includes Gö6983. In some embodiments, the ovotransferrin is derived from chicken or is the same as ovotransferrin present in yolk of an avian egg. For example, the avian egg is a chicken egg. For example, the LIF is chicken LIF.

In some embodiments, the inhibitor of activin receptor-like kinases-4, -5, and -7 includes SB431542. In some embodiments, the culture medium further includes N2B27 medium composed of Dulbecco's Modified Eagle Medium/Nutrient Mixture F-12 (DMEM/F12):Neuralbasal (1:1) plus 0.5×N2, 0.5×B27 supplement. In some embodiments, the culture medium further includes a basal medium including a solution of biological molecules such as fatty acids, lipids, amino acids, vitamins minerals, sugars and other relevant molecules. In some embodiments the basal medium is N2B27 medium composed of Dulbecco's Modified Eagle Medium/Nutrient Mixture F-12 (DMEM/F12):Neuralbasal (1:1) plus 0.5×N2, 0.5×B27 supplement.

In some embodiments, a concentration of the ovotransferrin is in a range of about 1.0 μg/ml to about 300 μg/ml. In some embodiments, a concentration of the ovotransferrin is in a range of about 5 μg/ml to about 60 μg/ml. In some embodiments, a concentration of the ovotransferrin is in a range of about 10 μg/ml to about 30 μg/ml. For example, the concentration of the ovotransferrin is about 20 μg/ml.

In some embodiments, in the culture medium, a concentration of IWR-1 is in a range of about 50 nM to about 30 μM; a concentration of Gö6983 is in a range of about 0.1 μM to about 40 μM; a concentration of SB431542 is in a range of about 50 μM to about 35 μM; and a concentration of chicken LIF is in a range of about 0.5 ng/ml to about 0.5 μg/ml. In some embodiments, in the culture medium, a concentration of IWR-1 is in a range of about 0.25 μM to about 6 μM; a concentration of Gö6983 is in a range of about 0.5 μM to about 8 μM; a concentration of SB431542 is in a range of about 250 μM to about 7 μM; and a concentration of chicken LIF is in a range of about 2.5 ng/ml to about 2.5 ng/ml. In some embodiments, in the culture medium, a concentration of IWR-1 is in a range of about 0.5 μM to about 3 μM; a concentration of Gö6983 is in a range of about 1 μM to about 4 μM; a concentration of SB431542 is in a range of about 0.5 μM to about 3.5p M; and a concentration of chicken LIF is in a range of about 5 ng/ml to about 50 ng/ml. For example, the concentration of IWR-1 is about 2.5 μM; the concentration of Gö6983 is about 2 μM; the concentration of SB431542 is about 2 μM; and the concentration of chicken LIF is about 10 ng/ml or about 40 ng/ml.

In some embodiments, the culture medium is used for derivation and/or propagation of ESCs from ostrich species.

According to various embodiments of the present disclosure, a culture medium includes a Wnt inhibitor; a protein kinase C (PKC) inhibitor; ovotransferrin; and one or more inhibitors of activin receptor-like kinases-4, -5, and/or -7. For example, the Wnt inhibitor includes Wnt/β-catenin signaling inhibitor IWR-1. For example, the PKC inhibitor includes Gö6983. For example, the ovotransferrin is derived from chicken or is the same as ovotransferrin present in yolk of an avian egg. In some embodiments of any of the aspects, the ovotransferrin can be recombinant ovotransferrin. For example, the inhibitor of activin receptor-like kinases-4, -5, and -7 includes SB431542. In some embodiments, the culture medium further includes N2B27 medium composed of Dulbecco's Modified Eagle Medium/Nutrient Mixture F-12 (DMEM/F12):Neuralbasal (1:1) plus 0.5×N2, 0.5×B27 supplement.

In some embodiments, a concentration of the ovotransferrin is in a range of about 0.1 μg/ml to about 300 μg/ml. In some embodiments, a concentration of the ovotransferrin is in a range of about 5 μg/ml to about 60 μg/ml. In some embodiments, a concentration of the ovotransferrin is in a range of about 10 μg/ml to about 30 μg/ml.

In some embodiments, the concentration of the ovotransferrin is about 20 μg/ml. In some embodiments, a concentration of IWR-1 is in a range of about 50 nM to about 30 μM; a concentration of Gö6983 is in a range of about 0.1 μM to about 40 μM; and a concentration of SB431542 is in a range of about 50 nM to about 35 μM. In some embodiments, the concentration of the ovotransferrin is about 20 μg/ml. In some embodiments, a concentration of IWR-1 is in a range of about 0.25 μM to about 6 μM; a concentration of Gö6983 is in a range of about 0.5 μM to about 8 M; and a concentration of SB431542 is in a range of about 0.25 μM to about 7 μM. In some embodiments, the concentration of the ovotransferrin is about 20 μg/ml. In some embodiments, a concentration of IWR-1 is in a range of about 0.5 μM to about 3 μM; a concentration of Gö6983 is in a range of about 1 μM to about 4 μM; and a concentration of SB431542 is in a range of about 0.5 μM to about 3.5 μM.

In some embodiments, the concentration of IWR-1 is about 2.5 μM; the concentration of Gö6983 is about 2 μM; and the concentration of SB431542 is about 2 μM.

It is further demonstrated herein that the methods and compositions for producing an oviparious stem cell are suitable for producing an oviparous induced pluripotent stem cell, when combined with contacting an oviparous cell, e.g., an avian cell, with reprogramming factors. iPSC induction for mammalian cells is known in the art and typically accomplished with retroviral transduction of cells. Oviparous cells, e.g., avian cells, can be contacted with reprogramming factors by any method known in the art. In some embodiments of any of the aspects, a oviparous cell which is to be reprogrammed is a lineage-restricted cell, e.g., a lineage-restricted somatic cell. In some embodiments of any of the aspects, a oviparous cell which is to be reprogrammed is a fibroblast. In some embodiments of any of the aspects, a oviparous cell which is to be reprogrammed is an embryonic fibroblast.

In one aspect of any of the embodiments, described herein is a method of producing an oviparous induced pluripotent stem cell, the method comprising contacting an oviparous cell (e.g., a lineage-restricted oviparous cell) with one or more of: Oct 4, Sox2, Sox3, Klf4, c-Myc, Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3. In some embodiments of any of the aspects, a method described herein can further comprise producing an iPSC, the producing comprising contacting an oviparous cell with one or more of: Oct 4, Sox2, Sox3, Klf4, c-Myc, Nanog, Noto, Cdx2, Lin28B, Gnot2, Gata4, Vgll1, Vgll2, Hand 1, Sp5, Otx2, Dbx2, Dmrt3, Tfap2A, Sp6, Eomes, and Tbx3.

In one aspect of any of the embodiments, described herein is a method of producing an oviparous induced pluripotent stem cell, the method comprising contacting an oviparous cell with:
a) Oct4;
b) Sox2 or Sox3;
c) Klf4; and
d) c-Myc; and optionally
e) one or more of Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3.

In some embodiments of any of the aspects, a method described herein can further comprise producing an iPSC, the producing comprising contacting an oviparous cell with:
a) Oct4;
b) Sox2 or Sox3;
c) Klf4; and
d) c-Myc; and optionally
e) one or more of Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3.

In one aspect of any of the embodiments, described herein is a method of producing an oviparous induced pluripotent stem cell, the method comprising contacting an oviparous cell with:
a) Oct4;
b) Sox2 or Sox3;
c) Klf4; and
d) c-Myc.

In some embodiments of any of the aspects, a method described herein can further comprise producing an iPSC, the producing comprising contacting an oviparous cell with:
a) Oct4;
b) Sox2 or Sox3;
c) Klf4; and
d) c-Myc.

In some embodiments of any of the aspects, the oviparous cell is contacted with Sox3. In some embodiments of any of the aspects, the oviparous cell is contacted with Sox3 and not contacted with Sox2. In some embodiments of any of the aspects, the oviparous cell is contacted with exogenous Oct4; Sox2 or Sox3; Klf4; and c-Myc. In some embodiments of any of the aspects, the oviparous cell is contacted with ectopic Oct4; Sox2 or Sox3; Klf4; and c-Myc. In some embodiments of any of the aspects, the Oct4; Sox2 or Sox3; Klf4; and c-Myc are oviparous Oct4; Sox2 or Sox3; Klf4; and c-Myc. In some embodiments of any of the aspects, the Oct4; Sox2 or Sox3; Klf4; and c-Myc are oviparous Oct4; Sox2 or Sox3; Klf4; and c-Myc from or of the same species as the oviparous cell. In some embodiments of any of the aspects, the Oct4; Sox2 or Sox3; Klf4; and c-Myc are avian Oct4; Sox2 or Sox3; Klf4; and c-Myc. In some embodiments of any of the aspects, the Oct4; Sox2 or Sox3; Klf4; and c-Myc are avian Oct4; Sox2 or Sox3; Klf4; and c-Myc from or of the same species as the avian cell. In some embodiments of any of the aspects, the Oct4; Sox2 or Sox3; Klf4; and c-Myc are fish Oct4; Sox2 or Sox3; Klf4; and c-Myc. In some embodiments of any of the aspects, the Oct4; Sox2 or Sox3; Klf4; and c-Myc are fish Oct4; Sox2 or Sox3; Klf4; and c-Myc from or of the same species as the fish cell.

In some embodiments of any of the aspects, the oviparous cell is an oviparous somatic cell. In some embodiments of any of the aspects, the oviparous somatic cell is a fibroblast or embryonic fibroblast. In some embodiments of any of the aspects, the avian cell is an avian somatic cell. In some embodiments of any of the aspects, the avian somatic cell is a fibroblast or embryonic fibroblast. In some embodiments of any of the aspects, the contacting an oviparous cell with:
a) Oct4;
b) Sox2 or Sox3;
c) Klf4; and
d) c-Myc; and optionally
e) one or more of Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3
   is performed concurrently with contacting the oviparous cell with at least one of: a Wnt inhibitor; a protein kinase C (PKC) inhibitor; and ovotransferrin as described herein. In some embodiments of any of the aspects, the contacting an oviparous cell with:
a) Oct4;
b) Sox2 or Sox3;
c) Klf4; and
d) c-Myc; and optionally
e) one or more of Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3
   is performed concurrently with contacting the oviparous cell with a composition comprising least one of: a Wnt inhibitor; a protein kinase C (PKC) inhibitor; and ovotransferrin as described herein.

In some embodiments of any of the aspects, a cell described herein is not contacted with a MEK inhibitor or a GSK3 inhibitor. In some embodiments of any of the aspects, a cell described herein is not contacted with a MEK inhibitor. In some embodiments of any of the aspects, a cell described herein is not contacted with a GSK3 inhibitor. In some embodiments of any of the aspects, a cell described herein is not contacted with a MEK inhibitor or a GSK3 inhibitor during a contacting or culturing step described herein. In some embodiments of any of the aspects, a composition or combination described herein does not comprise a MEK inhibitor or a GSK3 inhibitor.

In some embodiments of any of the aspects, an oviparous induced pluripotent stem cell produced according to the methods described herein expresses LMNA. In some embodiments of any of the aspects, an oviparous induced pluripotent stem cell produced according to the methods described herein expresses LTRA1. In some embodiments of any of the aspects, an oviparous induced pluripotent stem cell produced according to the methods described herein expresses PHLDA1. In some embodiments of any of the aspects, an oviparous induced pluripotent stem cell produced according to the methods described herein expresses FGF1. In some embodiments of any of the aspects, an oviparous induced pluripotent stem cell produced according to the methods described herein expresses GASK1B. In some embodiments of any of the aspects, an oviparous induced pluripotent stem cell produced according to the methods described herein expresses one or more of LMNA, LTRA1, PHLDA1, GFG1, and GASK1B. In some embodiments of any of the aspects, an oviparous induced pluripotent stem cell produced according to the methods described herein expresses LMNA, LTRA1, PHLDA1, GFG1, and GASK1B.

In some embodiments of any of the aspects, an oviparous stem cell produced, maintained, or expanded according to the methods described herein expresses LMNA. In some embodiments of any of the aspects, an oviparous stem cell produced, maintained, or expanded according to the methods described herein expresses LTRA1. In some embodiments of any of the aspects, an oviparous stem cell produced, maintained, or expanded according to the methods described herein expresses PHLDA1. In some embodiments of any of the aspects, an oviparous stem cell produced, maintained, or expanded according to the methods described herein expresses FGF1. In some embodiments of any of the aspects, an oviparous stem cell produced, maintained, or expanded according to the methods described herein expresses GASK1B. In some embodiments of any of the aspects, an oviparous stem cell produced, maintained, or expanded according to the methods described herein expresses one or more of LMNA, LTRA1, PHLDA1, GFG1, and GASK1B. In some embodiments of any of the aspects, an oviparous stem cell produced, maintained, or expanded according to the methods described herein expresses LMNA, LTRA1, PHLDA1, GFG1, and GASK1B.

In one aspect of any of the embodiments, described herein is an oviparous stem cell produced, maintained, or expanded according to a method described herein.

The stem cells produced, maintained, or expanded according to a method described herein can be utilized for diagnostics, therapeutics, biomanufacturing or the like. The stem cells can also be engineered, modified, and/or differentiated to provide cells useful for various purposes. Methods for differentiating or genetically engineering/modifying cells are well known in the art.

For example, in one aspect of any of the embodiments, described herein is a method of veterinary treatment, the method comprising administering a stem cell produced, maintained, or expanded according to a method described herein, or a cell differentiated from the stem cell, to a non-human animal. In one aspect of any of the embodiments, described herein is a method of preparing a veterinary therapeutic, the method comprising producing, maintain, or expanding a stem cell according to a method described herein, and optionally, differentiating a lineage restricted cell from the stem cell. In some embodiments of any of the aspects, the method further comprises genetically engineering or genetically modifying the stem cell or the cell differentiated from the stem cell.

In one aspect of any of the embodiments, described herein is a method of biomanufacturing, the method comprising purifying or collecting a biomaterial produced by a stem cell produced, maintained, or expanded according to a method described herein, or from a cell differentiated from the stem cell. In some embodiments of any of the aspects, the method further comprises engineering the stem cell, oviparous stem cell, oviparous blastodermal cell, or oviparous cell to produce the biomaterial. Biomaterials can be secreted by the stem cell. Non-limiting exemplary biomaterials include a cytokine, growth factor, hormone, collagen, or protein (e.g., egg or milk protein), and/or a recombinant protein or polypeptide.

In one aspect of any of the embodiments, described herein is method of cellular agriculture, the method comprising purifying or collecting a food material produced by a stem cell produced, maintained, or expanded according to a method described herein, or from a cell differentiated from the stem cell. The food material can be a meat tissue or other edible tissue comprising the stem cell or cells differentiated from the stem cell.

In one aspect of any of the embodiments, described herein is a method of somatic cell nuclear transfer, the method comprising transferring nuclear material from a stem cell produced, maintained, or expanded according to a method described herein to a somatic cell.

In one aspect of any of the embodiments, described herein is a method of producing a lineage restricted cell, the method comprising differentiating a stem cell produced, maintained, or expanded according to a method described herein to a lineage restricted cell. In some embodiments of any of the aspects, the lineage restricted cell is a myoblast, a preadipocyte, a chondrocyte, an osteoblast, an osteoclast, a fibroblast, a pericyte, a keratinocyte, a mesenchymal stem cell, a neuron, a haematopoetic stem cell, or a primordial germ cell.

In one aspect of any of the embodiments, described herein is a method of producing a chimeric or recombinant non-human animal, the method comprising contacting a blastoderm with a stem cell produced, maintained, or expanded according to a method described herein. In some embodiments of any of the aspects, the method further comprises engineering the stem cell, oviparous stem cell, oviparous blastodermal cell, or oviparous cell, e.g., genetically engineering.
in one aspect of any of the embodiments, described herein is a method comprising:
   producing an induced stem cell from an oviparous cell, optionally according to a method described herein;
   producing, maintaining, or expanding the induced stem cell according to a method described herein; and
   differentiating a lineage restricted cell from the induced stem cell, optionally into an organoid.
      In some embodiments of any of the aspects, the method further comprises measuring or observing a phenotype of the lineage restricted cell or organoid. In some embodiments of any of the aspects, the method further comprises genetically engineering or genetically modifying the stem cell produced, maintained, or expanded according to a method described herein, or the cell differentiated from the stem cell.

It is to be understood that the present compounds, compositions, articles, devices, and/or methods are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.” and “for example” set off lists of one or more non-limiting examples, instances, or illustrations.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The components, steps, features, objects, benefits and advantages which have been discussed are merely illustrative. None of them, nor the discussions relating to them, are intended to limit the scope of protection in any way. Numerous other embodiments are also contemplated. These include embodiments which have fewer, additional, and/or different components, steps, features, objects, benefits and advantages. These also include embodiments in which the components and/or steps are arranged and/or ordered differently.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The terms “stem cell” or “undifferentiated cell” as used herein, refer to a cell in an undifferentiated or partially differentiated state that has the property of self-renewal and has the developmental potential to differentiate into multiple cell types. A stem cell is capable of proliferation and giving rise to more such stem cells while maintaining its functional potential, e.g., while functionally maintaining its ontological potential for tissue lineage specification. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

The term “stem cells” refers to cells that have the ability to divide for indefinite periods, (i.e. self-renewal) and to give rise to virtually all of the tissues (e.g., of the avian or oviparous body), including specialized cells of the body and embryonic adnexa. The stem cells include pluripotent cells, which upon undergoing further specialization become multipotent progenitor cells that can give rise to cells of somatic and extraembryonic tissue lineages, including functional cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue.

As used herein, the terms “induced pluripotent stem cell” and “iPSC,” refer to a pluripotent cell artificially derived from a differentiated somatic cell. iPSCs are capable of self-renewal and differentiation into cell fate-committed stem cells as well as various types of mature cells.

As used herein, the term “blastodermal” refers to cells founds in a flat disc of embryonic epithelial tissue in the embryo of an oviparous species, e.g., an avian embryo.

In some embodiments of any of the aspects, the cells are primary cells. In some embodiments of any of the aspects, the cells are cell lines.

In some embodiments of any of the aspects, the cells are obtained from or derived from diseased primary cells or an animal or embryo having a disease affecting the cells. In some embodiments of any of the aspects, the cells can be genetically modified or engineered to express a reporter construct. The function of the reporter construct is to produce a detectable signal when a certain cellular activity or state occurs. In some embodiments of any of the aspects, a reporter construct can be used to quantify the concentration, strength, or activity of the cellular activity or state. In some embodiments of any of the aspects, the reporter component comprises a reporter gene, e.g, a gene expressing a detectable signal or label.

As used herein, a cell population that is “enriched” in a particular cell type refers to a population in which the relative proportion of cells of a particular type has increased in comparison with a previous population of cells (for example, in comparison with a population of cells prior to treatment with one or more of a Wnt inhibitor; a protein kinase C (PKC) inhibitor; and ovotransferrin).

As used herein, the term “self-renewal” refers to the ability of a stem cell to produce daughter stem cells with the same phenotype, characteristics and functional potential as the original stem cell. In particular, self-renewal, as used herein, is defined as the ability to continue proliferation while maintaining an undifferentiated multi-potent stem cell state.

As used herein, the phrases “preserve” or “maintain” multi-potency or pluripotency refer to a process by which the degree of multi-potency or pluripotency of a population of cells is preserved or renewed over a period of time. The degree of multi-potency or pluripotency of a population of cells describes the number and identity of differentiated cell types into which a population of cells can differentiate. For example, a population of cells exhibiting multi-potency that has been maintained over a period of two days ex vivo (e.g., in culture) is capable of differentiating into at least the same number of different cell types as the population was capable of differentiating into at the beginning of the cell culture period.

As used herein, the term “inhibitor” refers to any compound, natural or synthetic, which can antagonize or reduce the activity of a target protein or signaling pathway. An inhibitor can be, for example, a peptide, a protein, an antibody, a peptidomimetic, an amino acid, an amino acid analog, a polynucleotide, a polynucleotide analog, an aptamer, a nucleotide, a nucleotide analog, an organic or inorganic compound. An inhibitor may attenuate or prevent the activity of a target protein either directly or indirectly. Direct inhibition can be obtained, for instance, by binding to a protein and preventing the protein from interacting with an endogenous molecule (i.e. ligand), such as an enzyme, an ion, a hormone, a growth factor, a cytokine, a substrate, or other binding partner, thereby diminishing the activity of the protein. For instance, an inhibitor may bind an enzyme active site and sterically preclude binding of an endogenous substrate at this location, thus decreasing the enzymatic activity of the protein. Alternatively, indirect inhibition can be obtained, for instance, by binding to a protein that promotes the activity of a target protein by inducing a conformational change or catalyzing a chemical modification of the target protein. For instance, indirect inhibition of a target protein may be achieved by binding and inactivating a kinase that catalyzes the phosphorylation of, and thus activates, the target protein.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

All articles, patents, patent applications, and other publications that have been cited in this disclosure are incorporated herein by reference.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. Degenerate versions include both tri-nucleotide gene sequences that have been modified for translational efficiency of amino-acid incorporation into peptides or proteins from their RNA templates (i.e. codon-optimization) and their unmodified versions. The phrase nucleotide sequence that encodes a protein or an RNA may also include introns to the extent that the nucleotide sequence encoding the protein may in some version contain an intron(s).

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Illustrative embodiments are now described. Other embodiments may be used in addition or instead. Details that may be apparent to a person of ordinary skill in the art may have been omitted. Some embodiments may be practiced with additional components or steps and/or without all of the components or steps that are described.

The details of one or more embodiments of the invention are set forth in the accompanying drawings and the description herein. Other features, objects, and advantages of the invention will be apparent from the description and drawings, and from the claims.

In some embodiments, the present technology may be defined in any of the following numbered paragraphs:

1. A method of deriving embryonic stem cells (ESCs) from avian species, the method comprising:
culturing an embryo extracted from an avian egg in a culture medium to harvest cells from yolk of the avian egg;
dissociating cells from the cultured embryo;
isolating a morphologically undifferentiated ESC colony from the dissociated cells in a culture medium supplemented with a Wnt inhibitor and a protein kinase C (PKC) inhibitor; and culturing the isolated ESC colony in the presence of ovotransferrin, thereby deriving ESCs.2.
   The method of paragraph 1, wherein the derived ESCs are germline competent and pluripotent.

3. The method of paragraph 1 or paragraph 2, wherein the Wnt inhibitor comprises Wnt/0-catenin signaling inhibitor IWR-1.

4. The method of any one of paragraphs 1-3, wherein the PKC inhibitor comprises Gö6983.

5. The method of any one of paragraphs 1-4, wherein the ESCs derived in the presence of the Wnt inhibitor and the PKC inhibitor express Nanog mRNA.

6. The method of any one of paragraphs 3-5, further comprising promoting ESC self-renewal by culturing the ESCs in the presence of ovotransferrin, IWR-1, Gö6983, an inhibitor of activin receptor-like kinases-4, -5, and -7, and a leukemia inhibitory factor (LIF).

7. The method of paragraph 6, wherein the LIF is chicken LIF.

8. The method of paragraph 6, wherein the inhibitor of activin receptor-like kinases-4, -5, and -7 comprises SB431542.

9. The method of paragraph 6, wherein the ovotransferrin is same as ovotransferrin present in the yolk.

10. The method of paragraph 6, wherein a culture medium used for ESC maintenance comprises a serum-free medium comprising Dulbecco's Modified Eagle Medium/Nutrient Mixture F-12 (DMEM/F12), Neuralbasal, N2, B27, and ovotransferrin.

11. The method of any one of paragraphs 7-10, wherein ESCs are continuously passaged beyond 2 passages while remaining morphologically undifferentiated by supplementing with ovotransferrin, IWR-1, Gö6983, SB431542, and chicken LIF.

12. The method of any one of paragraphs 3-5, further comprising promoting ESC self-renewal by culturing the ESCs in the presence of ovotransferrin, IWR-1, Gö6983, an inhibitor of activin receptor-like kinases-4, -5, and -7.

13. The method of paragraph 12, wherein the inhibitor of activin receptor-like kinases-4, -5, and -7 comprises SB431542.

14. The method of paragraph 12, wherein the ovotransferrin is same as ovotransferrin present in the yolk.

15. The method of paragraph 12, wherein a culture medium used for ESC maintenance comprises a serum-free medium comprising Dulbecco's Modified Eagle Medium/Nutrient Mixture F-12 (DMEM/F12), Neuralbasal, N2, B27, and ovotransferrin.

16. The method of any one of paragraphs 12-15, wherein ESCs are continuously passaged beyond 2 passages while remaining morphologically undifferentiated by supplementing with ovotransferrin, IWR-1, Gö6983, and SB431542.

17. The method of any one of paragraphs 1-5 and paragraphs 12-16, wherein the avian species comprises ostrich,

18. The method of any one of paragraphs 1-5 and paragraphs 12-17, wherein derivation and long-term self-renewal of ESCs is supported by ovotransferrin, IWR-1, and Gö6983.

19. The method of any one of paragraphs 1-5, wherein the avian species comprises chicken, and derivation and long-term self-renewal of ESCs is supported by ovotransferrin, IWR-1, and Go6983.

20. The method of any one of paragraphs 1-5, wherein the avian species comprises chicken, quail, goose, or pheasant, and derivation and self-renewal of ESCs is supported at least 2 weeks after culturing the isolated ESC colony by ovotransferrin, IWR-1, Gö6983, SB431542, and chicken LIF.

21. The method of paragraph 20, wherein the avian species further comprises duck or turkey.

22. The method of paragraph 1, wherein ESCs are cultured at 38° C. in 5% CO₂ with daily medium change.

23. A culture medium, comprising:
a Wnt inhibitor;
a protein kinase C (PKC) inhibitor;
ovotransferrin;
one or more inhibitors of activin receptor-like kinases-4, -5, and/or -7; and a leukemia inhibitory factor (LIF).

24. The culture medium of paragraph 23, wherein the Wnt inhibitor comprises Wnt/β-catenin signaling inhibitor IWR-1.

25. The culture medium of paragraph 23 or paragraph 24, wherein the PKC inhibitor comprises Gö6983.

26. The culture medium of any one of paragraphs 23-25, wherein the ovotransferrin is derived from chicken or is the same as ovotransferrin present in yolk of an avian egg.

27. The culture medium of any one of paragraphs 23-26, wherein the LIF is chicken LIF.

28. The culture medium of any one of paragraphs 23-27, wherein the inhibitor of activin receptor-like kinases-4, -5, and -7 comprises SB431542.

29. The culture medium of any one of paragraphs 23-28, further comprising N2B27 medium composed of Dulbecco's Modified Eagle Medium/Nutrient Mixture F-12 (DMEM/F12):Neuralbasal (1:1) plus 0.5×N2, 0.5×B27 supplement.

30. The culture medium of any one of paragraphs 23-29, wherein a concentration of the ovotransferrin is in a range of about 10 μg/ml to about 30 μg/ml.

31. The culture medium of paragraph 30, wherein the concentration of the ovotransferrin is about 20 μg/ml.

32. The culture medium of any one of paragraphs 29-31, wherein:
a concentration of IWR-1 is in a range of about 0.5 μM to about 3 μM;
a concentration of Gö6983 is in a range of about 1 μM to about 4 μM;
a concentration of SB431542 is in a range of about 0.5 μM to about 3.5 μM; and
a concentration of chicken LIF is in a range of about 5 ng/ml to about 50 ng/ml.

33. The culture medium of paragraph 32, wherein:
the concentration of IWR-1 is about 2.5 μM;
the concentration of Gö6983 is about 2 μM;
the concentration of SB431542 is about 2 μM; and
the concentration of chicken LIF is about 10 ng/ml or about 40 ng/ml.

34. The culture medium of any one of paragraphs 23-33, wherein the culture medium is used for derivation and/or propagation of ESCs from chicken, quail, goose, pheasant, duck and/or turkey.

35. A culture medium, comprising:
a Wnt inhibitor;
a protein kinase C (PKC) inhibitor;
ovotransferrin; and
one or more inhibitors of activin receptor-like kinases-4, -5, and/or -7.

36. The culture medium of paragraph 35, wherein the Wnt inhibitor comprises Wnt/β-catenin signaling inhibitor IWR-1.

37. The culture medium of paragraph 35 or paragraph 36, wherein the PKC inhibitor comprises Gö6983.

38. The culture medium of any one of paragraphs 35-37, wherein the ovotransferrin is derived from chicken or is the same as ovotransferrin present in yolk of an avian egg.

39. The culture medium of any one of paragraphs 35-38, wherein the inhibitor of activin receptor-like kinases-4, -5, and -7 comprises SB431542.

40. The culture medium of any one of paragraphs 35-39, further comprising N2B27 medium composed of Dulbecco's Modified Eagle Medium/Nutrient Mixture F-12 (DMEM/F12):Neuralbasal (1:1) plus 0.5×N2, 0.5×B27 supplement.

41. The culture medium of any one of paragraphs 35-40, wherein a concentration of the ovotransferrin is in a range of about 10 μg/ml to about 30 μg/ml.

42. The culture medium of paragraph 41, wherein the concentration of the ovotransferrin is about 20 μg/ml.

43. The culture medium of any one of paragraphs 35-42, wherein:
a concentration of IWR-1 is in a range of about 0.5 μM to about 3 μM;
a concentration of Gö6983 is in a range of about 1 μM to about 4 μM; and a concentration of SB431542 is in a range of about 0.5 μM to about 3.5 μM.

44. The culture medium of paragraph 43, wherein:
the concentration of IWR-1 is about 2.5 μM;
the concentration of Gö6983 is about 2 μM; and the concentration of SB431542 is about 2 μM.

45. The culture medium of any one of paragraphs 35-44, wherein the culture medium is used for derivation and/or propagation of ESCs from ostrich species.

In some embodiments, the present technology may be defined in any of the following numbered paragraphs:

1. A method of deriving oviparous embryonic stem cells (ESCs), the method comprising:
culturing an embryo extracted from an extracorporeal egg in a culture medium to harvest cells from the germinal disc of the vitelline membrane within the extracoporeal egg;
dissociating cells from the cultured embryo;
isolating a morphologically undifferentiated ESC colony from the dissociated cells in a culture medium supplemented with a Wnt inhibitor and a protein kinase C (PKC) inhibitor; and culturing the isolated ESC colony in the presence of ovotransferrin, thereby deriving ESCs.

2. The method of paragraph 1, wherein the derived ESCs are germline competent and pluripotent.

3. The method of paragraph 1 or paragraph 2, wherein the Wnt inhibitor comprises Wnt/0-catenin signaling inhibitor IWR-1.

4. The method of any one of paragraphs 1-3, wherein the PKC inhibitor comprises Gö6983.

5. The method of any one of paragraphs 1-4, wherein the ESCs derived in the presence of the Wnt inhibitor and the PKC inhibitor express Nanog mRNA.

6. The method of any one of paragraphs 3-5, further comprising promoting ESC self-renewal by culturing the ESCs in the presence of ovotransferrin, IWR-1, Gö6983, an inhibitor of activin receptor-like kinases-4, -5, and -7, and a leukemia inhibitory factor (LIF).

7. The method of paragraph 6, wherein the LIF is chicken LIF.

8. The method of paragraph 6, wherein the LIF is homologous to the species of the cultivated ESCs.

9. The method of any one of paragraphs 6-8, wherein the inhibitor of activin receptor-like kinases-4, -5, and -7 comprises SB431542.

10. The method of any one of paragraphs 6-9, wherein the ovotransferrin is same as ovotransferrin present in the yolk.

11. The method of any one of paragraphs 6-10, wherein a culture medium used for ESC maintenance comprises a basal medium

12. The method of any one of paragraphs 6-11, wherein a culture medium used for ESC maintenance comprises a serum-free medium, e.g., Dulbecco's Modified Eagle Medium/Nutrient Mixture F-12 (DMEM/F12), Neuralbasal, N2, B27, and ovotransferrin.

13. The method of any one of paragraphs 6-12, wherein ESCs are continuously passaged beyond 2 passages while remaining morphologically undifferentiated by supplementing with ovotransferrin, IWR-1, Gö6983, SB431542, and LIF.

14. The method of any one of paragraphs 3-5, further comprising promoting ESC self-renewal by culturing the ESCs in the presence of ovotransferrin, IWR-1, Gö6983, an inhibitor of activin receptor-like kinases-4, -5, and -7.

15. The method of any one of paragraphs 6-14, wherein the inhibitor of activin receptor-like kinases-4, -5, and -7 comprises SB431542.

16. The method of any one of paragraphs 6-15, wherein the ovotransferrin is homologous to the ovotransferrin present in the yolk.

17. The method of any one of the preceding paragraphs, wherein a culture medium used for ESC maintenance comprises a serum-free medium comprising Dulbecco's Modified Eagle Medium/Nutrient Mixture F-12 (DMEM/F12), Neuralbasal, N2, B27, and ovotransferrin.

18. The method of any one of the preceding paragraphs, wherein ESCs are continuously passaged beyond 2 passages while remaining morphologically undifferentiated by supplementing with ovotransferrin, IWR-1, Gö6983, and SB431542.

19. The method of any one of the preceding paragraphs, wherein the oviparous species is avian.

20. The method of any one of the preceding paragraphs, wherein the avian species comprises ostrich.

21. The method of any one of the preceding paragraphs, wherein derivation and long-term self-renewal of ESCs is supported by ovotransferrin, IWR-1, and Gö6983.

22. The method of any one of paragraphs 19-21, wherein the avian species comprises chicken, and derivation and long-term self-renewal of ESCs is supported by ovotransferrin, IWR-1, and Gö6983.

23. The method of any one of paragraphs 19-22, wherein the avian species comprises chicken, quail, goose, or pheasant, and derivation and self-renewal of ESCs is supported at least 2 weeks after culturing the isolated ESC colony by ovotransferrin, IWR-1, Gö6983, SB431542, and LIF.

24. The method of any one of paragraphs 19-23, wherein the avian species is duck or turkey.

25. The method of any one of the preceding paragraphs, wherein ESCs are cultured at 38° C. in 5% CO₂ with daily medium change.

26.A method of deriving oviparous induced pluripotent stem cells (iPSCs), the method compromising:
culturing oviparous somatic or lineage-restricted cells;
ectopically contacting the oviparous somatic or lineage-restricted cells with one or more
transcription factors selected from the group consisting of:
   Pou5f3 (Oct4), Sox3, Nanog, Noto, Cdx2, Klf4, Lin28B, Gnot2, Gata4, Vgll1, Vgll2,
   Hand1, Sp5, Otx2, Dbx2, Dmrt3, Tfap2A, Sp6, Eomes, Tbx3; and culturing contacted cells until they are reprogrammed to provide oviparous IPSCs.

27. A culture medium, comprising:
a Wnt inhibitor;
a protein kinase C (PKC) inhibitor;
ovotransferrin;
one or more inhibitors of activin receptor-like kinases-4, -5, and/or -7; and
a leukemia inhibitory factor (LIF).

28. The culture medium of paragraph 27, wherein the Wnt inhibitor comprises Wnt/β-catenin signaling inhibitor IWR-1.

29. The culture medium of paragraph 27 or paragraph 28, wherein the PKC inhibitor comprises Gö6983.

30. The culture medium of any one of paragraphs 27-29, wherein the ovotransferrin is derived from chicken.

31. The culture medium of any one of paragraphs 27-30, wherein the ovotransferrin is homologous to the ovotransferrin present in yolk of an egg of the species from which the cells were derived.

32. The culture medium of any one of paragraphs 27-31, wherein the LIF is chicken LIF.

33. The culture medium of any one of paragraphs 27-32, wherein the LIF is at least 70%, at least 80%, at least 85%, at least 90%, at least 95%, or 100% identical to the LIF of the species from which the ESCs were derived

34. The culture medium of any one of paragraphs 27-33, wherein the inhibitor of activin receptor-like kinases-4, -5, and -7 comprises SB431542.

35. The culture medium of any one of paragraphs 27-34, further comprising basal cell culture medium.

36. The culture medium of any one of paragraphs 27-35, further comprising a serum free basal cell culture medium.

37. The culture medium of any one of paragraphs 27-36, further comprising N2B27 medium composed of Dulbecco's Modified Eagle Medium/Nutrient Mixture F-12 (DMEM/F12):Neuralbasal (1:1) plus 0.5×N2, 0.5×B27 supplement.

38. The culture medium of any one of paragraphs 27-37, wherein a concentration of the ovotransferrin is in a range of about 0,1 μg/ml to about 300 μg/ml.

39. The culture medium of paragraph 38, wherein the concentration of the ovotransferrin is about 20 μg/ml.

40. The culture medium of any one of paragraphs 27-38, wherein:
a concentration of IWR-1 is in a range of about 50 nM to about 30 μM;
a concentration of Gö6983 is in a range of about 0.1 μM to about 40 μM;
a concentration of SB431542 is in a range of about 50 nM to about 35 μM; and
a concentration of LIF is in a range of about 0.5 ng/ml to about 500 ng/ml.

41. The culture medium of paragraph 40, wherein:
the concentration of IWR-1 is about 2.5 μM;
the concentration of Gö6983 is about 2 μM;
the concentration of SB431542 is about 2 μM; and
the concentration of LIF is about 10 ng/ml or about 40 ng/ml.

42. The culture medium of any one of paragraphs 27-41, wherein the culture medium is used for derivation and/or propagation of ESCs from chicken, quail, goose, pheasant, duck and/or turkey.

43. A culture medium, comprising:
a Wnt inhibitor;
a protein kinase C (PKC) inhibitor;
ovotransferrin; and
one or more inhibitors of activin receptor-like kinases-4, -5, and/or -7.

44. The culture medium of paragraph 43, wherein the Wnt inhibitor comprises Wnt/β-catenin signaling inhibitor IWR-1.

45. The culture medium of paragraph 35 or paragraph 36, wherein the PKC inhibitor comprises Gö6983.

46. The culture medium of any one of paragraphs 43-45, wherein the ovotransferrin is derived from chicken or is homologous to the ovotransferrin present in yolk of the egg from which the cultured embryonic cells were derived.

47. The culture medium of any one of paragraphs 43-46, wherein the inhibitor of activin receptor-like kinases-4, -5, and -7 comprises SB431542.

48. The culture medium of any one of paragraphs 43-47, further comprising, a basal cell culture medium, a serum-free basal cell culture medium, and/or N2B27 medium composed of Dulbecco's Modified Eagle Medium/Nutrient Mixture F-12 (DMEM/F12):Neuralbasal (1:1) plus 0.5×N2, 0.5×B27 supplement.

49. The culture medium of any one of paragraphs 43-48, wherein a concentration of the ovotransferrin is in a range of about 0.1 μg/ml to about 300 μg/ml.

50. The culture medium of paragraph 49, wherein the concentration of the ovotransferrin is about 20 μg/ml.

51. The culture medium of any one of paragraphs 43-50, wherein:
a concentration of IWR-1 is in a range of about 50 nM to about 30 μM;
a concentration of Gö6983 is in a range of about 0.1 μM to about 40 μM; and
a concentration of SB431542 is in a range of about 50 nM to about 35 μM.

52. The culture medium of paragraph 51, wherein:
the concentration of IWR-1 is about 2.5 μM;
the concentration of Gö6983 is about 2 μM; and
the concentration of SB431542 is about 2 μM.

53. The culture medium of any one of paragraphs 43-52, wherein the culture medium is used for derivation and/or propagation of ESCs from ostrich species.

54. The culture medium of any one of paragraphs 43-53, wherein the culture medium is used for derivation and/or propagation of ESCs from non-avian oviparous species.

In some embodiments, the present technology may be defined in any of the following numbered paragraphs:
1. A method of producing, maintaining, or expanding oviparous stem cells, the method comprising:
   contacting at least one oviparous stem cell or oviparous blastodermal cell with at least one of:
      a Wnt inhibitor; a protein kinase C (PKC) inhibitor; and ovotransferrin.
2. The method of paragraph 1, wherein the contacting comprises contacting the at least one oviparous stem cell or oviparous blastodermal cell with a cell culture medium comprising at least one of: a Wnt inhibitor; a protein kinase C (PKC inhibitor); and ovotransferrin.
3. The method of any one of the preceding paragraphs, wherein the contacting comprises contacting the at least one oviparous stem cell or avian blastodermal cell with a cell culture medium comprising at least one of: a Wnt inhibitor; a protein kinase C (PKC inhibitor); and egg extract comprising ovotransferrin.
4. The method of any one of the preceding paragraphs, wherein the at least one oviparous stem cell or oviparous blastodermal cell is contacted with a Wnt inhibitor and a PKC inhibitor.
5. The method of any one of the preceding paragraphs, wherein the at least one oviparous stem cell or oviparous blastodermal cell is contacted with a Wnt inhibitor and ovotransferrin.
6. The method of any one of the preceding paragraphs, wherein the at least one oviparous stem cell or oviparous blastodermal cell is contacted with a PKC inhibitor and ovotransferrin.
7. The method of any one of the preceding paragraphs, wherein the at least one oviparous stem cell or oviparous blastodermal cell is contacted with a Wnt inhibitor; a PKC inhibitor; and ovotransferrin.
8. The method of any one of the preceding paragraphs, wherein the Wnt inhibitor is selected from the group consisting of:
   IWR-1, E7449, AZ6102, WIKI4, 53AH, C59, XAV-939, ICG-001, triptonide, IWP-2, M2912 (MSC2504877), CCT251545, KY02111, M2912, Box5, KY05009, Heparan sulfate, Tetramethylthiuram disulfide (TMTD), JW55, MN64, RK-287107, G007-LK, and adavivint (SM04690).
9. The method of any one of the preceding paragraphs, wherein the Wnt inhibitor is IWR-1.
10. The method of any one of the preceding paragraphs, wherein the PKC inhibitor is selected from the group consisting of:
   Go6983, Go7874, Go6976, LY317615, GSK590693, HA-1077, Ro320432, Calophostin C, AEB071 (sotrastaurin), NSC-301739 (mitoxantrone), AM-2282 (staurosporine), GF109203X (bisindolylmaleimide I), Ro 31-8220 (bisindolylmaleimide IX), daphnetin, dequalinium chlrodie, A-3 hydrochloride, N-desmethyltamoxifen, bisindolylmaleimide VIII, H-1152, HA-100, rottlerin, bisindolylmaleimide IV, VTX-27, valrubicin, PKC-theta inhibitor, ML-7, LY333531, PKC412, NSC 646662, WAY-301398, epsilon-VI-2, chelerythrine, hypocrellin A, hypericin, oncrasin-1, LXS-196, NAS-301, 2-methoxy-1,4-naphthoquinone, quercetin, myricitrin, and methyl-hesperidin.
11. The method of any one of the preceding paragraphs, wherein the PKC inhibitor is Go6983.
12. The method of any one of the proceeding paragraphs, wherein the ovotrasferrin is an oviparous ovotransferrin.
13. The method of any one of the preceding paragraphs, wherein the ovotransferrin is an avian ovotransferrin.
14. The method of any one of the preceding paragraphs, wherein the ovotransferrin is a Gallus gallus ovotransferrin.
15. The method of any one of the preceding paragraphs, wherein the ovotransferrin is a Gallus gallus domesticus ovotransferrin.
16. The method of any one of the preceding paragraphs, wherein the ovotransferrin has a sequence homology of at least 70% to SEQ ID NO: 1.
17. The method of any one of the preceding paragraphs, wherein the ovotransferrin has a sequence homology of at least 70% to an endogenous ovotransferrin encoded in a genome of the at least one oviparous stem cell or oviparous blastodermal cell.
18. The method of any one of the preceding paragraphs, wherein the ovotransferrin has a sequence at least 80% identical to an endogenous ovotransferrin encoded in a genome of the at least one oviparous stem cell or oviparous blastodermal cell.
19. The method of any one of the proceeding paragraphs wherein contacting the oviparous stem cell or oviparous blastodermal cell with ovotransferrin comprises ectopically expressing the ovotransferrin in the oviparous stem cell or oviparous blastodermal cell.
20. The method of any one of the proceeding paragraphs wherein contacting the oviparous stem cell or oviparous blastodermal cell with ovotransferrin comprises culturing the oviparous stem cell or oviparous blastodermal cell with a feeder cell expressing the ovotransferrin.
21. The method of any one of the preceding paragraphs, wherein the at least one oviparous stem cell or oviparous blastodermal cell is further contacted with an activin receptor-like kinase 4, -5, and -7 inhibitor.
22. The method of any one of the preceding paragraphs, wherein the at least one oviparous stem cell or oviparous blastodermal cell is further contacted with an activin receptor-like kinase 4, -5, and -7 inhibitor while being contacted with at least one of a Wnt inhibitor; a protein kinase C (PKC inhibitor); and ovotransferrin.
23. The method of any one of paragraphs 21-22, wherein the activin receptor-like kinase 4, -5, and -7 inhibitor is SB431542.
24. The method of any one of the preceding paragraphs, wherein the at least one oviparous stem cell or oviparous blastodermal cell is further contacted with a LIF polypeptide.
25. The method of paragraph 24, wherein the at least one oviparous stem cell or oviparous blastodermal cell is further contacted with the LIF polypeptide while being contacted with at least one of a Wnt inhibitor; a protein kinase C (PKC inhibitor); and ovotransferrin.
26. The method of any one of paragraphs 24-25, wherein the at least one oviparous stem cell or oviparous blastodermal cell is further contacted with a LIF polypeptide while being contacted with the activin receptor-like kinase 4, -5, and -7 inhibitor and at least one of a Wnt inhibitor; a protein kinase C (PKC inhibitor); and ovotransferrin.
27. The method of any one of paragraphs 24-26, wherein the LIF polypeptide is an oviparous LIF polypeptide.
28. The method of any one of paragraphs 24-27, wherein the LIF polypeptide is an avian LIF polypeptide.
29. The method of any one of paragraphs 24-28, wherein the LIF polypeptide is an Gallus gallus LIF polypeptide.
30. The method of any one of paragraphs 24-29, wherein the LIF polypeptide is an Gallus gallus domesticus LIF polypeptide.
31. The method of any one of paragraphs 24-30, wherein the LIF has a sequence homology of at least 70% to SEQ ID NO: 2.
32. The method of any one of paragraphs 24-31, wherein the LIF has a sequence homology of at least 70% to an endogenous LIF encoded in a genome of the at least one oviparous stem cell or oviparous blastodermal cell.
33. The method of any one of paragraphs 24-32, wherein the LIF has a sequence at least 80% identical to an endogenous LIF encoded in a genome of the at least one oviparous stem cell or oviparous blastodermal cell.
34. The method of any one of paragraphs 24-33, wherein contacting the oviparous stem cell or oviparous blastodermal cell with LIF comprises ectopically expressing the LIF in the oviparous stem cell or oviparous blastodermal cell.
35. The method of any one of paragraphs 24-34, wherein contacting the oviparous stem cell or oviparous blastodermal cell with LIF comprises culturing the oviparous stem cell or oviparous blastodermal cell with a feeder cell expressing the LIF.
36. The method of any one of the preceding paragraphs, wherein the at least one oviparous stem cell or oviparous blastodermal cell is further contacted with egg yolk ex ovo.
37. The method of any one of the preceding paragraphs, wherein the at least one oviparous stem cell or oviparous blastodermal cell is further contacted with oviparous egg yolk ex ovo while being contacted with at least one of a Wnt inhibitor; a protein kinase C (PKC inhibitor); and ovotransferrin.
38. The method of any one of paragraphs 36-37, wherein the egg yolk is a 50-100 kDa fraction of egg yolk.
39. The method of any one of the preceding paragraphs, wherein the at least one oviparous stem cell or oviparous blastodermal cell is further contacted with egg extract ex ovo.
40. The method of any one of the preceding paragraphs, wherein the oviparous stem cells or at least one oviparous stem cell is an embryonic stem cell (ESC), an induced pluripotent stem cell (iPSC), multipotent stem cell, or pluripotent stem cell.
41. The method of any one of the preceding paragraphs, wherein the at least one oviparous stem cell is a reprogrammed somatic cell or extra-embryonic cell.
42. The method of any one of the preceding paragraphs, wherein the oviparous stem cells, at least one oviparous stem cell, or at least one oviparous blastodermal cell is an avian cell(s).
43. The method of any one of the preceding paragraphs, wherein the oviparous stem cells, at least one oviparous stem cell, or at least one oviparous blastodermal cell is a fish cell(s).
44. The method of any one of the preceding paragraphs, wherein the oviparous stem cells, at least one oviparous stem cell, or at least one oviparous blastodermal cell is a reptile cell(s).
45. The method of any one of the preceding paragraphs, further comprising a step of isolating an oviparous blastodermal cell from an oviparous embryo prior to contacting the at least one oviparous blastodermal cell with at least one of a Wnt inhibitor; a protein kinase C (PKC inhibitor); and ovotransferrin.
46. The method of 45, wherein the oviparous embryo is an EGK.X to EGK.XII stage avian embryo.
47. The method of any one of paragraphs 1-44, wherein the at least one oviparous stem cell is an induced pluripotent stem cell and the method further comprises a step of producing an induced pluripotent stem cell from an oviparous lineage-restricted cell prior to contacting the at least one oviparous stem cell with at least one of a Wnt inhibitor; a protein kinase C (PKC inhibitor); and ovotransferrin.
48. The method of paragraph 47, wherein the producing comprises contacting the at least one lineage-restricted cell with one or more of: Oct 4, Sox2, Sox3, Klf4, c-Myc, Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3.
49. The method of any one of paragraphs 47-48, wherein the lineage-restricted cell is a lineage-restricted somatic cell.
50. The method of any one of paragraph 47-49, wherein the lineage-restricted cell is a fibroblast.
51. The method of paragraph 50, wherein the fibroblast is an embryonic fibroblast.
52. The method of any one of paragraphs 47-51, wherein the producing step occurs concurrently with contacting the lineage-restricted cell with at least one of: a Wnt inhibitor; a protein kinase C (PKC) inhibitor; and ovotransferrin.
53. The method of any one of the preceding paragraphs, wherein the oviparous stem cell or oviparous blastodermal cell is a Gallus gallus, Meleagris gallopavo, Phasianus colchicus; Coturnix coturnix; a Galliformes; Anas platyrhynchos, Anser anser, an Anseriformes; Struthio camelus, a Struthioformes. Gallus gallus domesticus, a Phasiandiae; an Anatinae; Meleagris gallopavo domesticus; or Struthio camelus cell.
54. The method of any one of the preceding paragraphs, whereby germline competent and pluripotent stem cells are produced, maintained, or expanded.
55. The method of any one of the preceding paragraphs, whereby morphologically undifferentiated stem cells are produced, maintained, or expanded.
56. The method of any one of the preceding paragraphs, whereby stem cells expressing Nanog mRNA are produced, maintained, or expanded.
57. The method of any one of the preceding paragraphs, whereby stem cells having less than 5% methyl-cytosine (as a percentage of total cytosine content) are produced, maintained or expanded.
58. The method of any one of the preceding paragraphs, whereby stem cells having less than 4% methyl-cytosine (as a percentage of total cytosine content) are produced, maintained or expanded.
59. The method of any one of the preceding paragraphs, whereby stem cells having less than 3% methyl-cytosine (as a percentage of total cytosine content) are produced, maintained or expanded.
60. The method of any one of the preceding paragraphs, wherein the cells are cultured in a basal medium.
61. The method of any one of the preceding paragraphs, wherein the cells are cultured in a serum-free basal medium.
62. The method of any one of the preceding paragraphs, wherein the ovotransferrin is at a concentration of from about 0.1 μg/ml to about 300 μg/ml.
63. The method of paragraph 39, wherein the ovotransferrin is at a concentration of about 20 μg/ml.
64. The method of any one of the preceding paragraphs, wherein:
   a) the Wnt inhibitor is at a concentration of about 50 nM to about 30 μM;
   b) the PKC inhibitor is at a concentration of about 0.1 μM to about 40 μM;
   c) the activin receptor like kinase 4, -5, and -7 inhibitor is at a concentration of about 50 nM to about 35 μM; and/or
   d) the LIF polypeptide is at a concentration of about 0.5 ng/ml to about 500 ng/ml.
65. The method of any one of the preceding paragraphs, wherein:
   a) the Wnt inhibitor is at a concentration of about 2.5 μM;
   b) the PKC inhibitor is at a concentration of about 2 μM;
   c) the activin receptor like kinase 4, -5, and -7 inhibitor is at a concentration of about 2.5 μM; and
   d) the LIF polypeptide is at a concentration of about 10 ng/ml or about 40 ng/ml.
66. The method of any one of the preceding paragraphs, wherein the contacting step is conducted for at least one passage.
67. The method of any one of the preceding paragraphs, wherein the contacting step is conducted for at least two passages.
68. The method of any one of the preceding paragraphs, wherein the contacting step is conducted for at least two weeks.
69. The method of any one of the preceding paragraphs, wherein the contacting step comprises culturing the at least one oviparous stem cell or oviparous blastodermal cell with daily medium changes.
70. The method of any one of the preceding paragraphs, wherein the contacting step comprises culturing the at least one oviparous stem cell or oviparous blastodermal cell at 38° C. in 5% CO₂ with daily medium changes.
71. A composition or combination comprising:
   at least one Wnt inhibitor;
   at least one protein kinase C (PKC) inhibitor; and
   ovotransferrin.
72. The composition or combination of paragraph 71, comprising at least one Wnt inhibitor and at least one PKC inhibitor.
73. The composition or combination of paragraph 71, comprising at least one Wnt inhibitor and ovotransferrin.
74. The composition or combination of paragraph 71, comprising at least one PKC inhibitor and ovotransferrin.
75. The composition or combination of paragraph 71, comprising at least one Wnt inhibitor, at least one PKC inhibitor, and ovotransferrin.
76. The composition or combination of any one of paragraphs 71-75, wherein the Wnt inhibitor is selected from the group consisting of:
   IWR-1, E7449, AZ6102, WIKI4, 53AH, C59, XAV-939, ICG-001, triptonide, IWP-2, M2912 (MSC2504877), CCT251545, KY02111, M2912, Box5, KY05009, Heparan sulfate, Tetramethylthiuram disulfide (TMTD), JW55, MN64, RK-287107, G007-LK, and adavivint (SM04690).
77. The composition or combination of any one of paragraphs 71-75, wherein the Wnt inhibitor is IWR-1.
78. The composition or combination of any one of paragraphs 71-77, wherein the PKC inhibitor is selected from the group consisting of:
Go6983, Go7874, Go6976, LY317615, GSK590693, HA-1077, Ro320432, Calophostin C, AEB071 (sotrastaurin), NSC-301739 (mitoxantrone), AM-2282 (staurosporine), GF109203X (bisindolylmaleimide I), Ro 31-8220 (bisindolylmaleimide IX), daphnetin, dequalinium chlrodie, A-3 hydrochloride, N-desmethyltamoxifen, bisindolylmaleimide VIII, H-1152, HA-100, rottlerin, bisindolylmaleimide IV, VTX-27, valrubicin, PKC-theta inhibitor, ML-7, LY333531, PKC412, NSC 646662, WAY-301398, epsilon-VI-2, chelerythrine, hypocrellin A, hypericin, oncrasin-1, LXS-196, NAS-301, 2-methoxy-1,4-naphthoquinone, quercetin, myricitrin, and methyl-hesperidin.
79. The composition or combination of any one of paragraphs 71-77, wherein the PKC inhibitor is Go6983.
80. The composition or combination of any one of paragraphs 71-79, further comprising at least one oviparous stem cell or oviparous blastodermal cell.
81. The composition or combination of any one of paragraphs 71-80, wherein the ovotrasferrin is an oviparous ovotransferrin.
82. The composition or combination of any one of paragraphs 71-81, wherein the ovotransferrin is an avian ovotransferrin.
83. The composition or combination of any one of paragraphs 71-82, wherein the ovotransferrin is a Gallus gallus ovotransferrin.
84. The composition or combination of any one of paragraphs 71-83, wherein the ovotransferrin is a Gallus gallus domesticus ovotransferrin.
85. The composition or combination of any one of paragraphs 71-84, wherein the ovotransferrin has a sequence homology of at least 70% to SEQ ID NO: 1.
86. The composition or combination of any one of paragraphs 80-85, wherein the ovotransferrin has a sequence homology of at least 70% to an endogenous ovotransferrin encoded in a genome of the at least one oviparous stem cell or oviparous blastodermal cell.
87. The composition or combination of any one of paragraphs 80-86, wherein the ovotransferrin has a sequence at least 80% identical to an endogenous ovotransferrin encoded in a genome of the at least one oviparous stem cell or oviparous blastodermal cell.
88. The composition or combination of any one of paragraphs 80-79, wherein the oviparous stem cell or oviparous blastodermal cell comprises a construct, vector, or modification ectopically expressing the ovotransferrin.
89. The composition or combination of any one of paragraphs 71-88, further comprising a feeder cell expressing the ovotransferrin.
90. The composition or combination of any one of paragraphs 71-89, further comprising at least one activin receptor-like kinase 4, -5, and/or -7 inhibitor.
91. The composition or combination of paragraph 90, wherein the activin receptor-like kinase 4, -5, and -7 inhibitor is SB431542.
92. The composition of combination of any one of paragraphs 71-91, further comprising a leukemia inhibitory factor (LIF) polypeptide.
93. The composition of combination of paragraph 92, wherein the LIF polypeptide is an oviparous LIF polypeptide.
94. The composition of combination of any one of paragraphs 92-93, wherein the LIF polypeptide is an avian LIF polypeptide.
95. The composition of combination of any one of paragraphs 92-94, wherein the LIF polypeptide is an Gallus gallus LIF polypeptide.
96. The composition of combination of any one of paragraphs 92-95, wherein the LIF polypeptide is an Gallus gallus domesticus LIF polypeptide.
97. The composition of combination of any one of paragraphs 92-96, wherein the LIF has a sequence homology of at least 70% to SEQ ID NO: 2.
98. The composition of combination of any one of paragraphs 92-97, wherein the LIF has a sequence homology of at least 70% to an endogenous LIF encoded in a genome of the at least one oviparous stem cell or oviparous blastodermal cell.
99. The composition of combination of any one of paragraphs 92-98, wherein the LIF has a sequence at least 80% identical to an endogenous LIF encoded in a genome of the at least one oviparous stem cell or oviparous blastodermal cell.
100. The composition of combination of any one of paragraphs 92-99, wherein the the oviparous stem cell or oviparous blastodermal cell comprises a construct, vector, or modification ectopically expressing the LIF.
101. The composition of combination of any one of paragraphs 92-100, further comprising a feeder cell expressing the LIF.
102. The composition of combination of any one of paragraphs 71-101, further comprising egg yolk.
103. The composition of combination of paragraph 102, wherein the egg yolk is a 50-100 kDa fraction of egg yolk.
104. The composition of combination of any one of paragraphs 71-101, further comprising egg extract.
105. The composition or combination of any one of paragraphs 71-104, further comprising a cell culture medium.
106. The composition or combination of paragraph 105, wherein the cell culture medium comprises a basal medium.
107. The composition or combination of paragraph 105, wherein the cell culture medium comprises a serum-free basal medium.
108. The composition of combination of any one of paragraphs 71-107, wherein the at least one oviparous stem cell is an embryonic stem cell (ESC), an induced pluripotent stem cell (iPSC), multipotent stem cell, or pluripotent stem cell.
109. The composition of combination of any one of paragraphs 71-108, wherein the at least one oviparous stem cell is a reprogrammed somatic cell or extra-embryonic cell.
110. The composition of combination of any one of paragraphs 71-109, wherein the oviparous stem cells, at least one oviparous stem cell, or at least one oviparous blastodermal cell is an avian cell(s).
111. The composition of combination of any one of paragraphs 71-109, wherein the oviparous stem cells, at least one oviparous stem cell, or at least one oviparous blastodermal cell is a fish cell(s).
112. The composition of combination of any one of paragraphs 71-109, wherein the oviparous stem cells, at least one oviparous stem cell, or at least one oviparous blastodermal cell is a reptile cell(s).
113. The composition of combination of any one of paragraphs 71-112, further comprising one or more of: Oct 4, Sox2, Sox3, Klf4, c-Myc, Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3.
114. The composition of combination of any one of paragraphs 71-113, wherein the oviparous stem cell or oviparous blastodermal cell is a Gallus gallus, Meleagris gallopavo, Phasianus colchicus; Coturnix coturnix; a Galliformes; Anas platyrhynchos, Anser anser, an Anseriformes; Struthio camelus, a Struthioformes. Gallus gallus domesticus, a Phasiandiae; an Anatinae; Meleagris gallopavo domesticus; or Struthio camelus cell.
115. The composition of combination of any one of paragraphs 71-114, wherein the ovotransferrin is at a concentration of from about 0.1 μg/ml to about 300 μg/ml.
116. The composition of combination of paragraph 115, wherein the ovotransferrin is at a concentration of about 20 μg/ml.
117. The composition of combination of any one of paragraphs 71-116, wherein:
   a) the Wnt inhibitor is at a concentration of about 50 nM to about 30 μM;
   b) the PKC inhibitor is at a concentration of about 0.1 μM to about 40 μM;
   c) the activin receptor like kinase 4, -5, and -7 inhibitor is at a concentration of about 50 nM to about 35 μM; and/or
   d) the LIF polypeptide is at a concentration of about 0.5 ng/ml to about 500 ng/ml.
118. The composition of combination of any one of paragraphs 71-117, wherein:
   a) the Wnt inhibitor is at a concentration of about 2.5 μM;
   b) the PKC inhibitor is at a concentration of about 2 μM;
   c) the activin receptor like kinase 4, -5, and -7 inhibitor is at a concentration of about 2.5 μM; and
   d) the LIF polypeptide is at a concentration of about 10 ng/ml or about 40 ng/ml.
119. A method of producing an avian induced pluripotent stem cell, the method comprising contacting an oviparous cell with one or more of: Oct 4, Sox2, Sox3, Klf4, c-Myc, Nanog, Noto, Cdx2, Lin28B, Gnot2, Gata4, Vgll1, Vgll2, Hand1, Sp5, Otx2, Dbx2, Dmrt3, Tfap2A, Sp6, Eomes, and Tbx3.
120. The method of paragraph 77, wherein the oviparous cell is a lineage-restricted somatic cell.
121. The method of paragraph 78, wherein the lineage-restricted somatic cell is a fibroblast.
122. The method of paragraph 79, wherein the fibroblast is an embryonic fibroblast.
123. The method of any one of paragraphs 77-80, wherein the producing step occurs concurrently with contacting the stem cell with a composition of any one of paragraphs 71-118.
124. A stem cell produced, maintained, or expanded according to any one of paragraphs 1-70.
125. A method of veterinary treatment, the method comprising administering a stem cell produced, maintained, or expanded according to any one of paragraphs 1-70, or a cell differentiated from the stem cell, to a non-human animal.
126. A method of preparing a veterinary therapeutic, the method comprising producing, maintain, or expanding a stem cell according to any one of paragraphs 1-70, and optionally, differentiating a lineage restricted cell from the stem cell.
127. The method of any one of paragraphs 125-126, further comprising genetically engineering or genetically modifying the stem cell produced, maintained, or expanded according to any one of paragraphs 1-70 or the cell differentiated from the stem cell.
128. A method of biomanufacturing, the method comprising purifying or collecting a biomaterial produced by a stem cell produced, maintained, or expanded according to any one of paragraphs 1-70, or from a cell differentiated from the stem cell.
129. The method of paragraph 128, further comprising engineering the stem cell, oviparous stem cell, oviparous blastodermal cell, or oviparous cell to produce the biomaterial.
130. The method of any one of paragraphs 128-129, wherein the biomaterial is secreted by the stem cell.
131. The method of any one of paragraphs 128-130, wherein the biomaterial is a cytokine, growth factor, hormone, collagen, or protein (e.g., egg or milk protein).
132. The method of any one of paragraphs 128-131, wherein the biomaterial is a recombinant protein or polypeptide.
133. A method of cellular agriculture, the method comprising purifying or collecting a food material produced by a stem cell produced, maintained, or expanded according to any one of paragraphs 1-70, or from a cell differentiated from the stem cell.
134. The method of paragraph 133, wherein the food material is a meat tissue comprising the stem cell or cells differentiated from the stem cell.
135. A method of somatic cell nuclear transfer, the method comprising transferring nuclear material from a stem cell produced, maintained, or expanded according to any one of paragraphs 1-70 to a somatic cell.
136. A method of producing a lineage restricted cell, the method comprising differentiating a stem cell produced, maintained, or expanded according to any one of paragraphs 1-70 to a lineage restricted cell.
137. The method of paragraph 136, wherein the lineage restricted cell is a myoblast, a preadipocyte, a chondrocyte, an osteoblast, an osteoclast, a fibroblast, a pericyte, a keratinocyte, a mesenchymal stem cell, a neuron, a haematopoetic stem cell, or a primordial germ cell.
138. A method of producing a chimeric or recombinant non-human animal, the method comprising contacting a blastoderm with a stem cell produced, maintained, or expanded according to any one of paragraphs 1-70.
139. The method of paragraph 138, wherein the method further comprises engineering the stem cell, oviparous stem cell, oviparous blastodermal cell, or oviparous cell.
140. A method comprising:
   producing an induced stem cell from an oviparous cell, optionally according to the method of any one of paragraphs 119-123;
   producing, maintaining, or expanding the induced stem cell according to any one of the methods of paragraphs 1-70; and
   differentiating a lineage restricted cell from the induced stem cell, optionally into an organoid.
141. The method of paragraph 140, further comprising measuring or observing a phenotype of the lineage restricted cell or ogranoid.
142. The method of any one of paragraphs 140-141, further comprising genetically engineering or genetically modifying the stem cell produced, maintained, or expanded according to any one of paragraphs 1-70, or the cell differentiated from the stem cell.

The technology described herein is further illustrated by the following examples which in no way should be construed as being further limiting.

### EXAMPLES

### Example 1

### Development of Culture Conditions for the Derivation of Chicken ESCs.

To develop conditions for the derivation of chicken ESCs, we plated blastodermal cells isolated from freshly laid Rhode Island Red chicken eggs (EGK.X stage) and screened small molecules, growth factors and cytokines that have been shown to promote self-renewal of mouse, rat, or human pluripotent stem cells (Table 1).

TABLE 1
List of compounds tested for culturing chicken ESCs.

| Name | Function |
|---|---|
| Leukemia Inhibitory | activate LIF/STAT3 signaling |
| Factor | |
| PD0325901 | MEK1/2 inhibitor |
| CHIR-99021 | GSK3 α/β inhibitor |
| IWR-1-endo | stabilize Axin-scaffolding destruction complexes |
| 53AH | inhibit Wnt signaling |
| C59 | inhibit porcupine/wnt signaling |
| XAV-939 | inhibit tankyrase1/2, wnt signaling |
| Gö6983 | pan-PKC inhibitor |
| Gö7874 | pan-PKC inhibitor |
| A-83-01 | inhibitor of TGF-β/nodal receptors (ALK5-TD; ALK4-TD; ALK7-TD) |
| PD173074 | FGFR1 inhibitor |
| Fetal bovine serum | activate multiple signalings |
| FGF4 | activate MAPK signaling |
| Vx-11e | ERK inhibitor |
| cyclopamine | Hedgehog signaling pathway antagonist of Smoothened |
| U0126 | MEK1/2 inhibitor |
| BIX02189 | MEK5 inhibitor |
| Vitamin B12 | a water soluble vitamin with a key role in the normal functioning of the brain |
| | and nervous system, and for the formation of blood. |
| Rapamycin | mTOR inhibitor |
| EGF | binds to EGFR |
| RepSox | inhibitor of TGFβR-1/ALK5 |
| HGF | activate ligand for the receptor tyrosine kinase |
| BMP4 | a TGF-β superfamily ligand |
| DMH1 | selective ALK2 inhibitor |
| SB431542 | selective ALK5 inhibitor |
| ICG-001 | antagonizes Wnt/β-catenin/TCF-mediated transcription and specifically binds |
| | to CREB-binding protein |
| Triptonide | inhibit Wnt/β-catenin signaling by targeting the downstream C-terminal |
| | transcription domain of β-catenin or a nuclear component associated with |
| | β-catenin |
| Triptolide | a NF-κB inhibitor with dual actions by disruption of p65/CBP interaction and |
| | by reduction of p65 protein; also activate Wnt/β-catenin signaling by |
| | degrading β-catenin protein |
| Pifithrin-μ | a specific p53 inhibitor |
| Retinoic acid | a ligand for both the retinoic acid receptor (RAR) and the retinoid X receptor |
| | (RXR) |
| DMH4 | selective VEGFR-2 inhibitor |
| Thrombopoietin | binds and activates an 68-78 kDa glycoprotein receptor belonging to the GH |
| | family of cytokine receptors |
| Hepatocyte growth | binds heparan-sulfate proteoglycans and the widely expressed receptor |
| factor | tyrosine kinase, HGF R/c-MET |
| PKCζ Pseudo- | inhibit an atypical type of Protein Kinase C ζ (zeta) |
| substrate inhibitor, | |
| Myristoylated (ZIP) | |
| Vascular endothelial | binds to VEGF receptor R1 and R2 |
| growth factor (VEGF) | |
| M-CSF | binds to M-CSF receptor |
| IFN alpha | a type I interferon |
| GM-CSF | binds to a heterodimeric receptor complex composed of GM-CSF R |
| | alpha/CD116 and beta chain (CD131) |
| Hsp90α | interact with proteins in their native conformation and help to |
| | maintain/stabilize ligand-bound states. |
| bpV (HOpic) | a potent inhibitor of PTEN |
| SP600125 | a broad-spectrum JNK inhibitor |
| SB203580 | a p38 MAPK inhibitor |
| Activin A | a member of TGF-β family of protein |
| DAPT | a γ-secretase inhibitor |
| 2-Deoxy-D-glucose | a glycolytic inhibitor |
| (2-DG) | |
| SKL2001 | agonist of Wnt/β-catenin pathway |
| iCRT14 | a β-catenin/Tcf inhibitor |
| Knockout Serum | a defined, FBS-free formulation designed to replace FBS |
| Replacement | |
| L-ascorbic acid | enzymatic cofactor, antioxidant |
| Wnt3a | activate Wnt signaling |
| IM-12 | a GSK3 inhibitor |
| SU5402 | a potent multi-targeted receptor tyrosine kinase inhibitor |
| IL-6 | signals through a receptor complex of IL-6 R alpha and gp130 |
| Forskolin | increases level of cAMP |
| TGF alpha | a member of the EGF family of cytokines |
| TGF beta1 | a member of the TGF-beta superfamily, binds to a type II ser/thr kinase |
| | receptor (TGF-beta RII) |
| acidic FGF | a 17 kDa nonglycosylated member of the FGF family of mitogenic peptides |
| TNF-alpha | binds to TNF R1 or TNF RII, leads to activation of NF-κB or MAPK |
| | signalings |
| SB590885 | a potent B-Raf inhibitor |
| WH-4-023 | a potent and orally active Lck/Src inhibitor |
| IKK-16 | a selective IκB kinase (IKK) inhibitor |
| SU6656 | a selective Src family kinase inhibitor |
| LY294002 | inhibit PI3Kα/δ/β |
| Triamterene | casein kinase 1 inhibitor; also blocks epithelial Na+ channel |
| IGF-1 | signals through dimeric IGF1R, dimeric Insulin R, or a heterodimer of the |
| | two receptors |
| Chicken serum | activates multiple signalings |

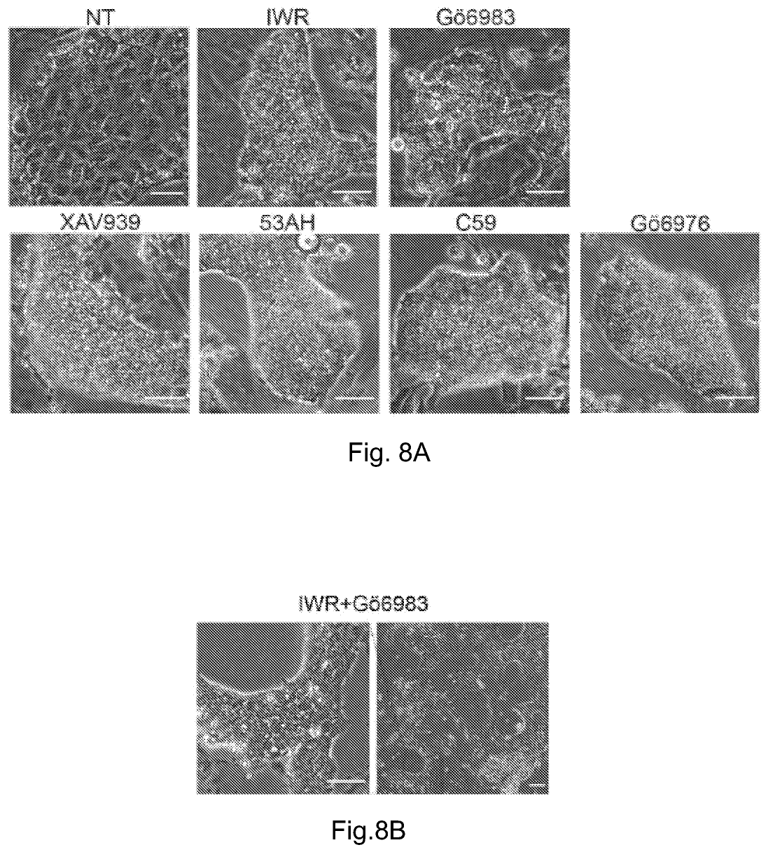

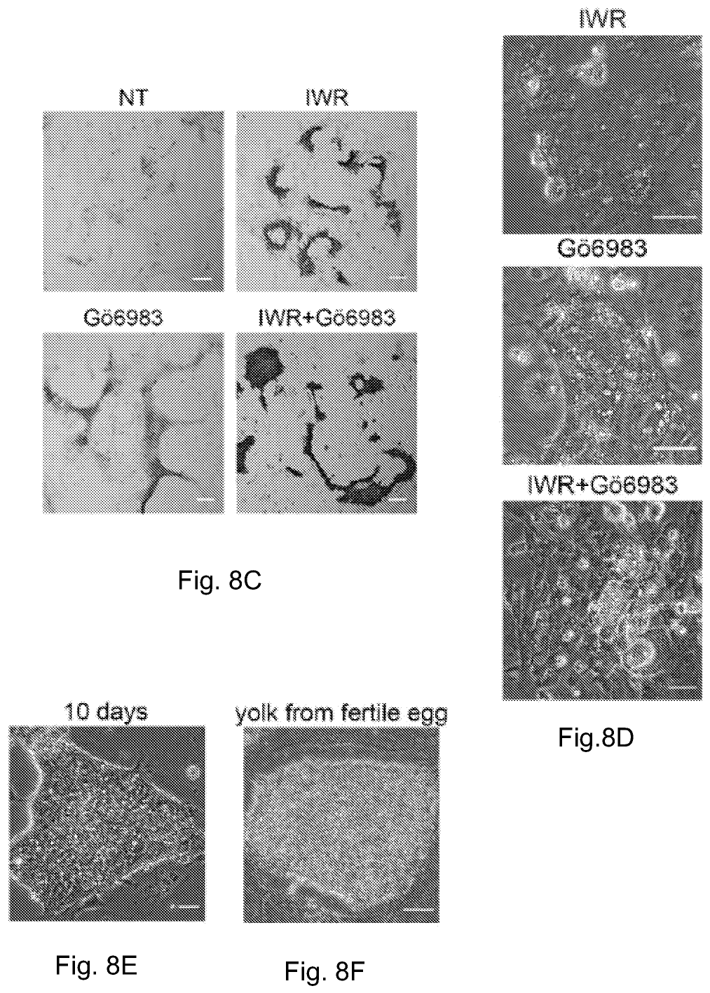

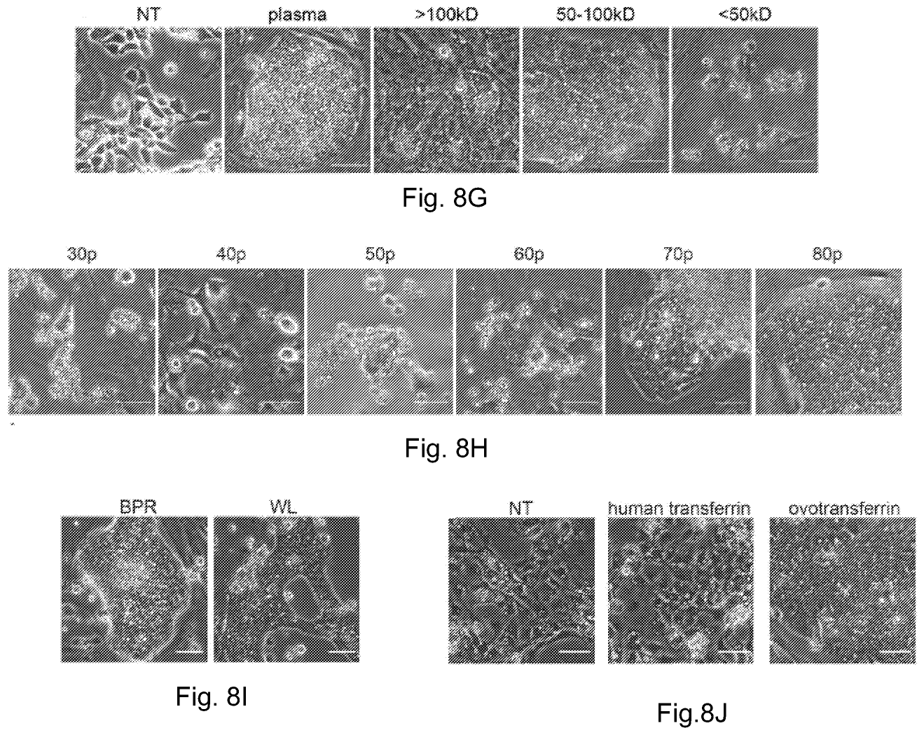

Morphologically undifferentiated ESC colonies emerged in culture supplemented with either Wnt/β-catenin signaling inhibitor IWR-1 or protein kinase C (PKC) inhibitor Gö6983 (FIG. 8A). We observed similar effects from other Wnt inhibitors and PKC inhibitors (FIG. 8A). When combined, IWR-1 and Gö6983 acted synergistically to promote chicken ESC self-renewal (FIG. 8B). Chicken ESCs derived in IWR-1 and Gö6983 expressed Nanog mRNA, a pluripotency marker (FIG. 8C). Nonetheless, chicken ESCs gradually differentiated after passaging and could not be maintained beyond 2 passages in the presence of IWR-1, Gö6983, or both (FIG. 8D).

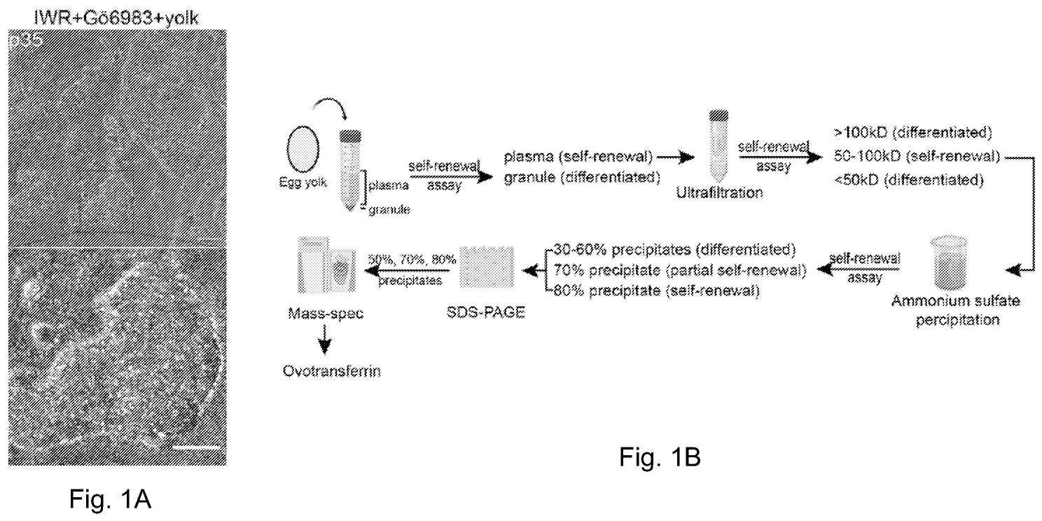

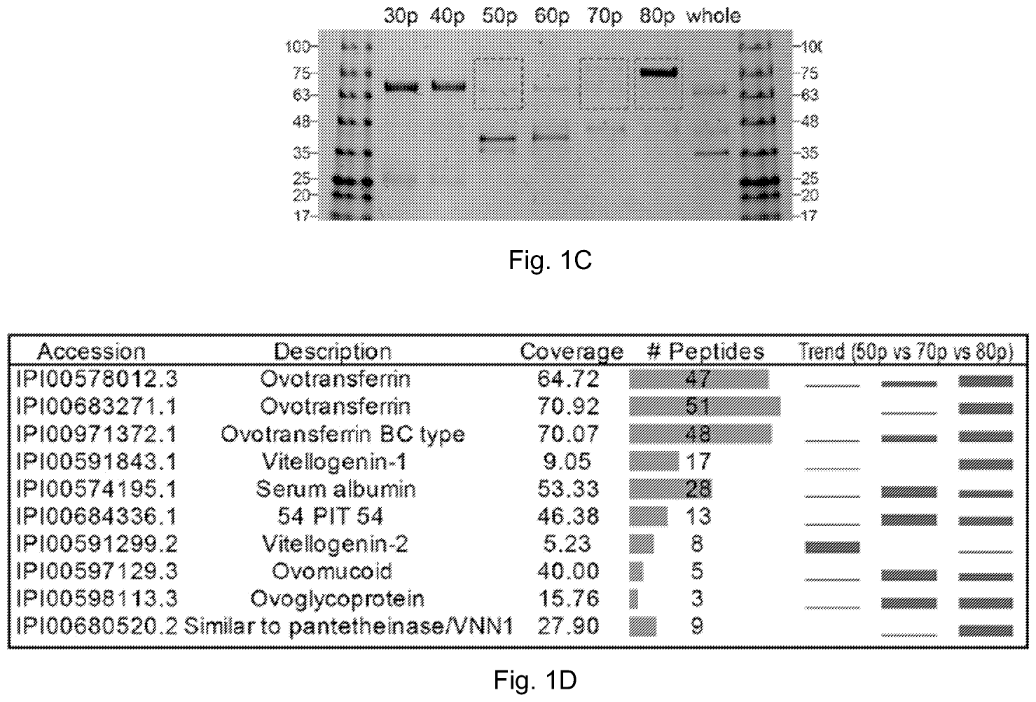

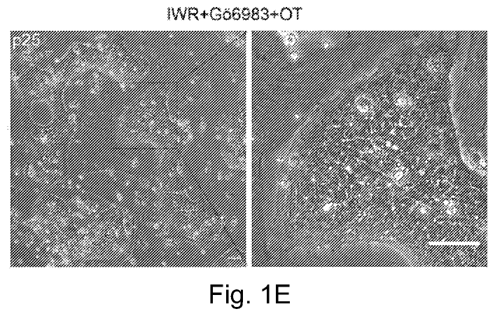

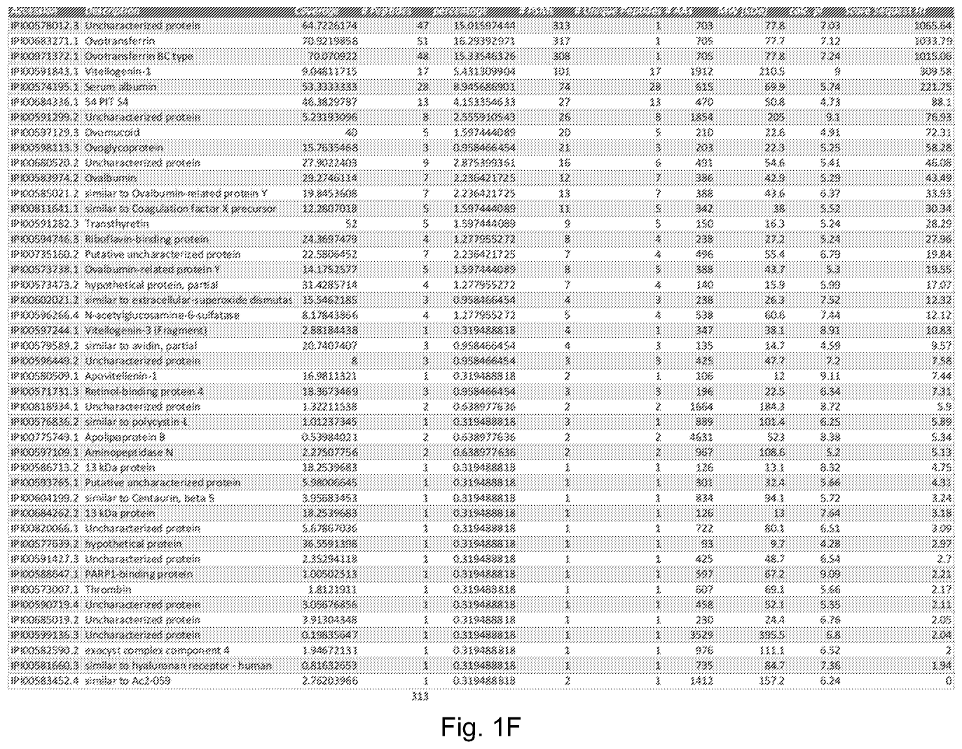

We noticed that chicken ESCs grew better when more egg yolk was carried over during the isolation of embryos. Chicken ESCs remained undifferentiated even 2 weeks after the initial plating if the medium containing residual yolk was left unchanged (FIG. 8E). This phenomenon implies that egg yolk might contain component(s) important for chicken ESC self-renewal. Indeed, chicken ESCs could be continuously passaged while remaining morphologically undifferentiated when supplemented with yolk, IWR-1, and Gö6983 (FIG. 1A). In some embodiments, 100% of the ESCs remain morphologically undifferentiated. In some embodiments, at least 90% of the ESCs remain morphologically undifferentiated. In some embodiments, at least 95% of the ESCs remain morphologically undifferentiated. Yolk from fertilized and unfertilized eggs had the same effect in promoting chicken ESC self-renewal (FIG. 8F).

Next, we sought to identify the ESC self-renewal-promoting component(s) in the yolk using the strategy illustrated in FIG. 1B. Yolk was fractionated into sedimented granules and plasma. Plasma, but not granules, promoted chicken ESC self-renewal, suggesting that the self-renewal-promoting component(s) were soluble (FIG. 8G). We used the molecular-weight cut-off (MWCO) membrane filters to fractionate plasma into three fractions: <50 kDa, 50-100 kDa, and >100 kDa. 50-100 kDa fraction, but not the other two fractions, retained the self-renewal-promoting effect (FIG. 8G). The 50-100 kDa fraction was further fractionated by ammonium sulfate precipitation. The resulting protein precipitates were sequentially recovered at the following ammonium sulfate concentrations: 30%, 40%, 50%, 60%, 70% and 80%. While precipitates from 70% (70p) could partially mimic the effect of yolk plasma, precipitates from 80% (80p) had the strongest effect for the maintenance of chicken ESCs (FIG. 8H). SDS-PAGE analysis revealed an enriched band with a size of ˜75 kDa in 80p (FIG. 1C). This band was identified to be ovo-transferrin by Mass spectrometry analysis (FIG. 1D and Table 2 in FIG. 1F).

Chicken ESCs from different breeds could be efficiently derived and maintained in the presence of ovotransferrin, IWR-1, and Gö6983 (OT/2i hereafter) (FIGS. 1E and 8I), suggesting that the major self-renewal-promoting component in egg yolk is ovo-transferrin. The function of ovotransferrin in OT/2i could not be replaced by its human ortholog, indicating the species-specific effect of ovotransferrin for

chicken ESC self-renewal and survival (FIG. 8J).

### Development of Universal Culture Conditions for the Derivation of Avian ESCs

We tested whether ESCs could be derived from other avian species in OT/2i. We isolated blastodermal cells from fertilized pheasant, duck and turkey eggs and cultured them in OT/2i. ESC colonies emerged 2-3 days after plating, but differentiated rapidly, mainly towards beating cardiomyocytes (FIG. 9A). We examined whether blocking signaling pathways involved in cardiomyocyte differentiation from pluripotent stem cells could improve ESC self-renewal. We found that addition of SB431542, a potent and selective inhibitor of activin receptor-like kinases-4, -5, and -7, in combination with OT/2i (OT/3i hereafter) prevented ESC differentiation towards beating cardiomyocytes (FIG. 9B). However, OT/3i was not sufficient to maintain quail ESC self-renewal long-term, and addition of chicken LIF is necessary (FIG. 9B). Notably, the self-renewal-promoting effect of chicken LIF on quail ESCs cannot be substituted by human LIF, possibly due to the low degree of sequence homology between avian and mammalian LIF (FIG. 9C).

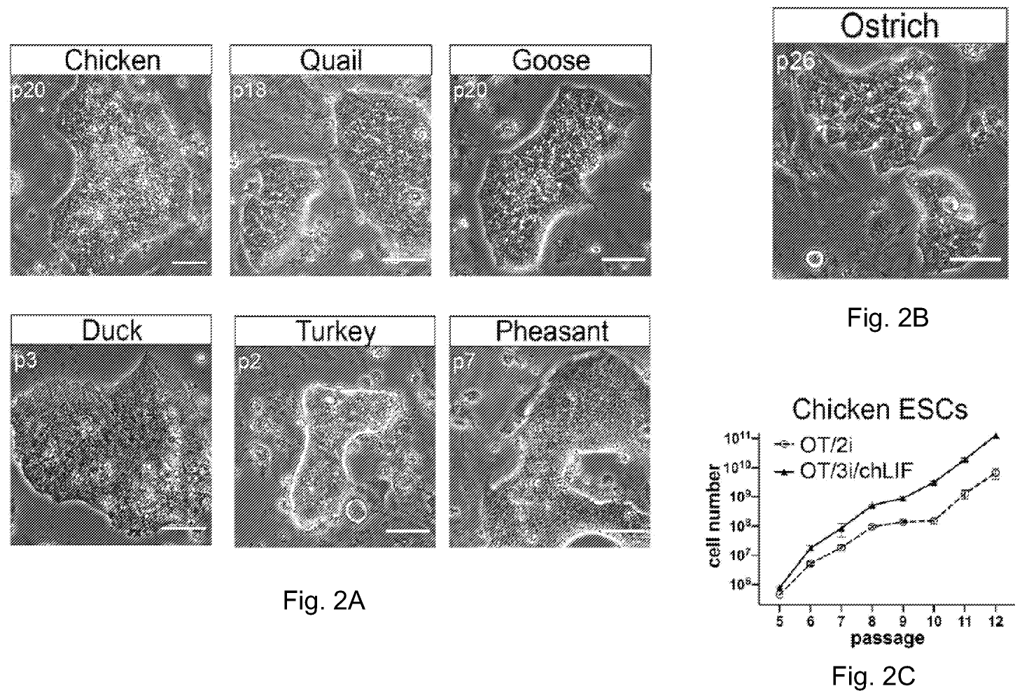

OT/3i/chLIF can support derivation and long-term self-renewal of ESCs from chicken, quail, goose, pheasant, but not ostrich (FIGS. 2A and 9D). Ostrich ESCs could be derived and maintained in OT/3i, and addition of chicken LIF induced differentiation (FIGS. 2B and 9D). Additionally, OT/3i/chLIF could also promote self-renewal of duck and turkey ESCs (FIG. 2A), even though we have not yet evaluated its long-term self-renewal effect in these ESCs.

We then compared OT/3i/chLIF with OT/2i for chicken ESC derivation and maintenance. Both conditions allowed efficient derivation (8 stable ESC lines established from 9 embryos in both conditions) and long term maintenance of chicken ESCs. Markedly, however, chicken ESCs in OT/3i/chLIF propagated more robustly than in OT/2i, suggesting that SB431542 (sometimes referred to as “SB43” herein) and chicken LIF can act synergistically with IWR-1 and Gö6983 to promote chicken ESC self-renewal (FIG. 2C). In some embodiments, 100% of the ESCs remain morphologically undifferentiated when supplemented with ovotransferrin, IWR-1, Gö6983, SB431542, and chicken LIF. In some embodiments, at least 90% of the ESCs remain morphologically undifferentiated. In some embodiments, at least 95% of the ESCs remain morphologically undifferentiated.

In chick embryo, some epiblast cells remain pluripotent during the initial stage of primitive streak formation (HH2 stage). We examined whether these pluripotent cells could be captured and maintained under chicken ESC culture conditions. We isolated cells from HH2-5 stage chick embryos and culture them in OT/2i, OT/3i, or OT/3i/chLIF. All the cells differentiated (FIG. 9E), suggesting that the time window during early chick development from which chicken ESCs can be established under the chicken ESC conditions is very narrow (EGK.X-XII stage).

### Chicken ESCs Derived and Maintained in OT/3i/chLIF are Pluripotent

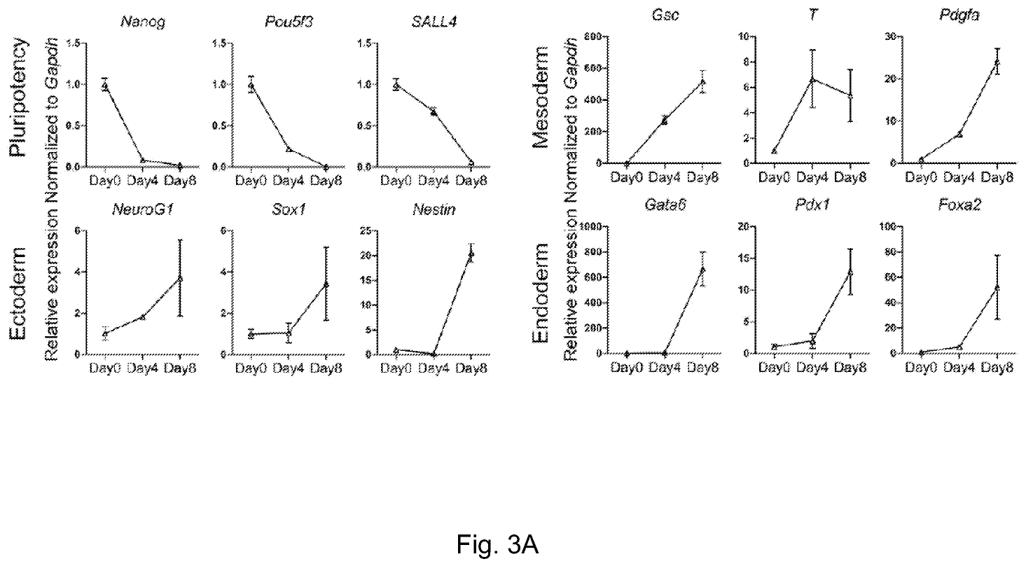

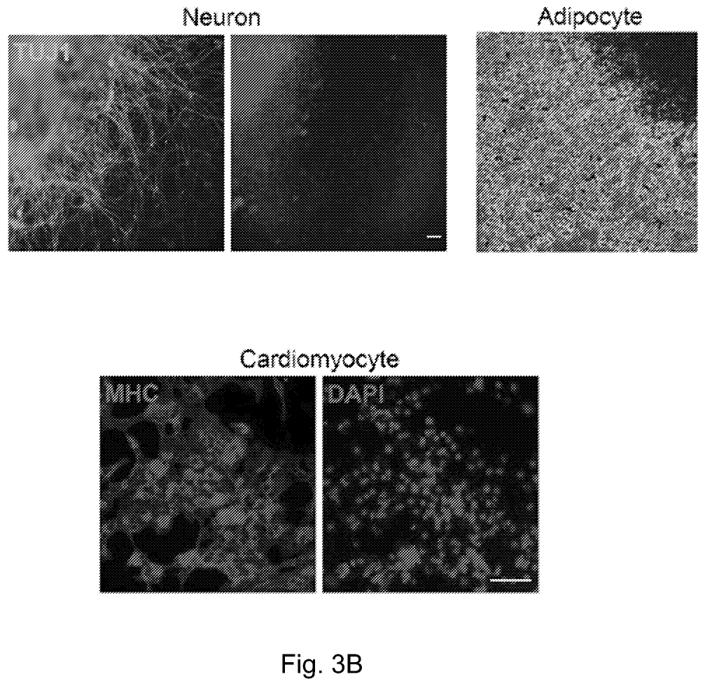

To examine whether chicken ESCs derived and maintained in OT/3i/chLIF are pluripotent, we induced differentiation of chicken ESCs through the formation of embryoid bodies (EBs). Within the EBs, ESCs undergo spontaneous differentiation towards all three germ layers. RT-qPCR analysis showed that during the process of EB formation from chicken ESCs, the expression of pluripotency genes Nanog, Pou5f3, and Sall4 was dramatically downregulated whereas the expression of ectoderm, endoderm, and mesoderm markers was significantly upregulated (FIG. 3A). Immunostaining further confirmed that TUJ1-positive neurons, Oil-red staining-positive adipocytes, and myosin heavy chain (MHC)-positive cardiomyocytes could be generated from chicken ESCs (FIG. 3B). These data suggest that chicken ESCs are pluripotent, retaining the capacity to differentiate into cells of all three germ layers in vitro.

### Transcriptome Comparison Shows Close Relatedness of Chicken ESCs to EGK.X Blastodermal Cells

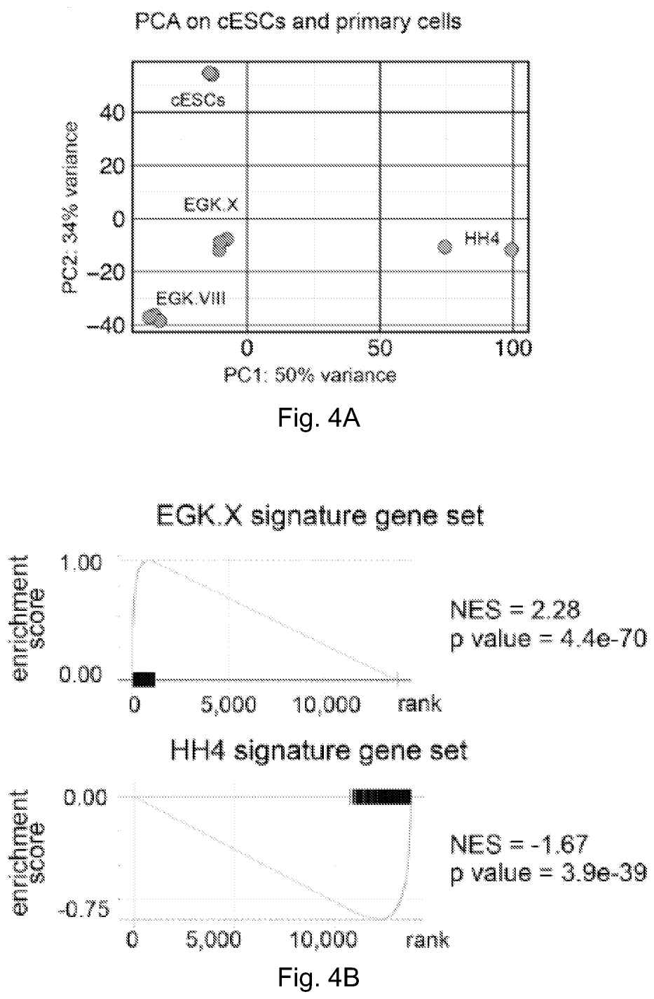

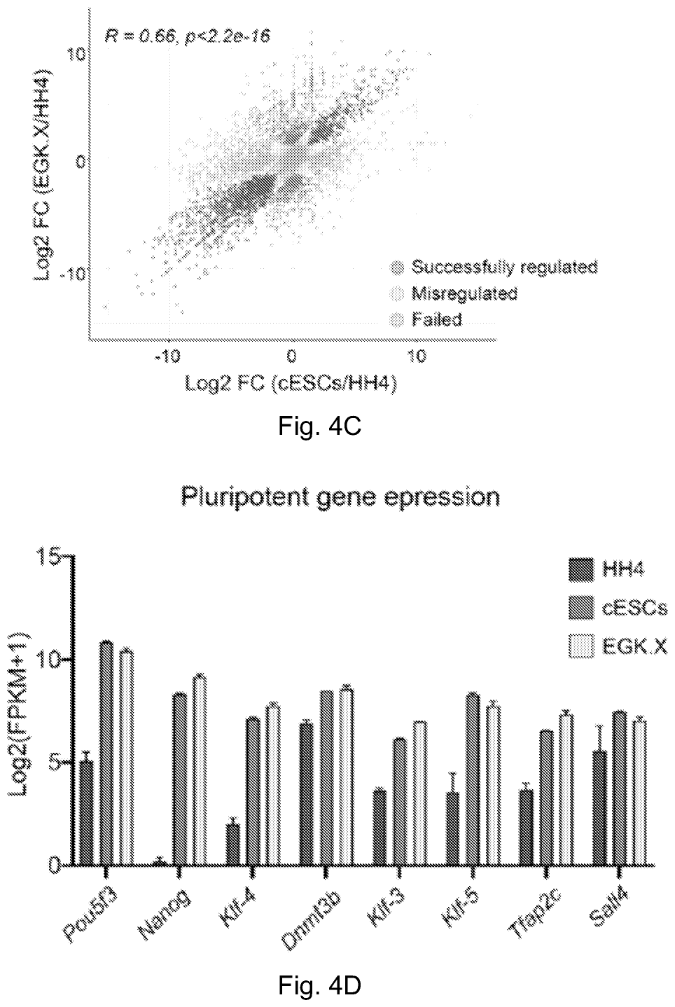

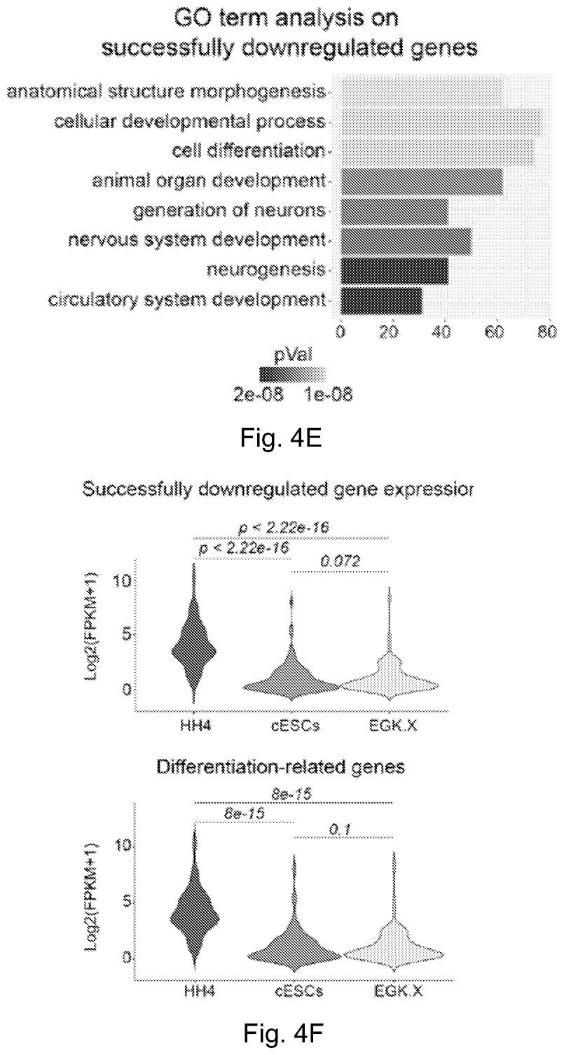

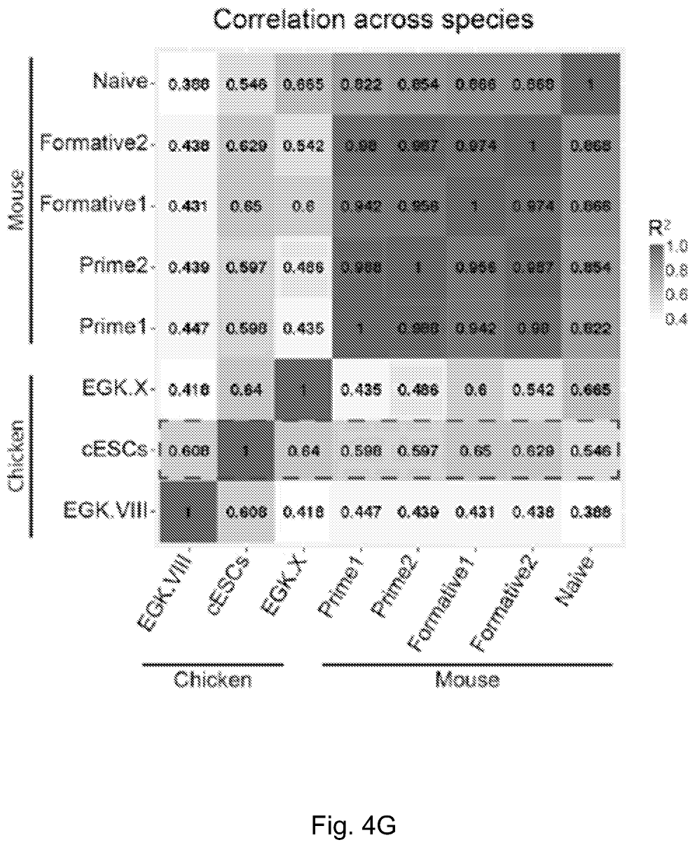

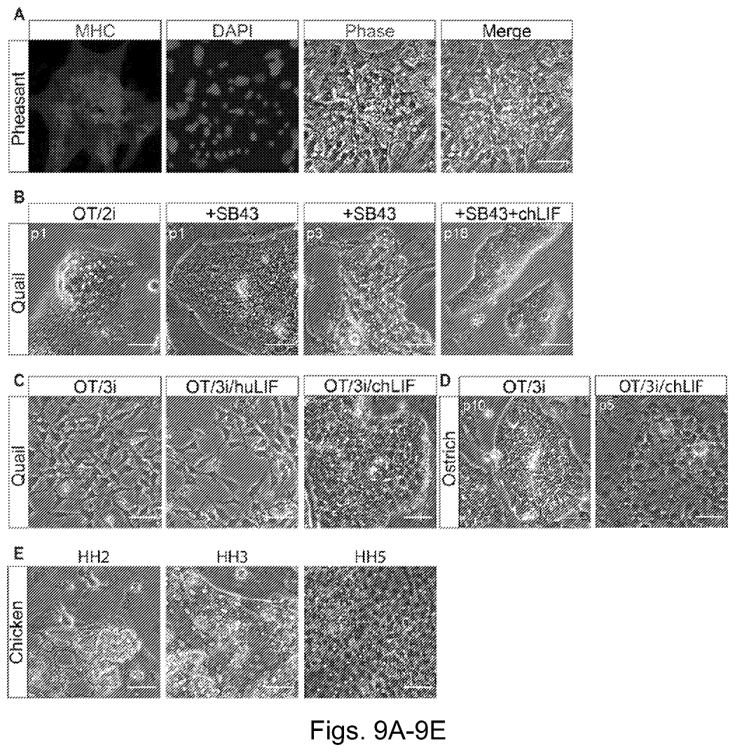

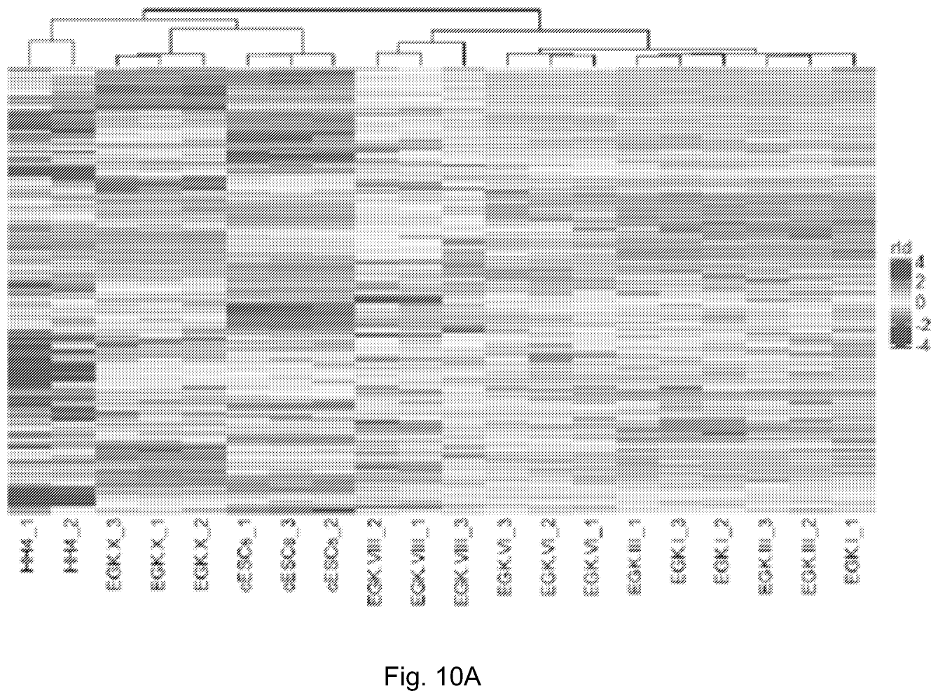

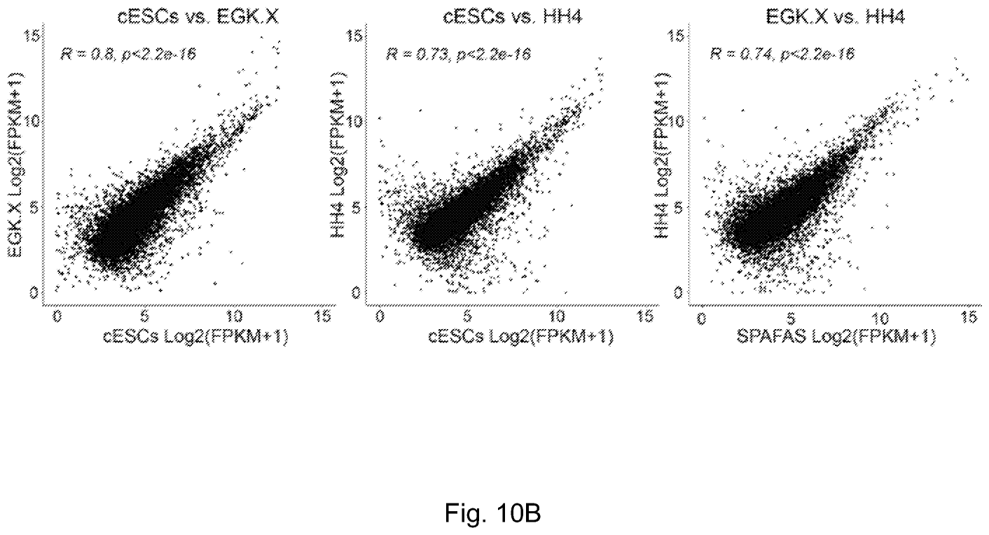

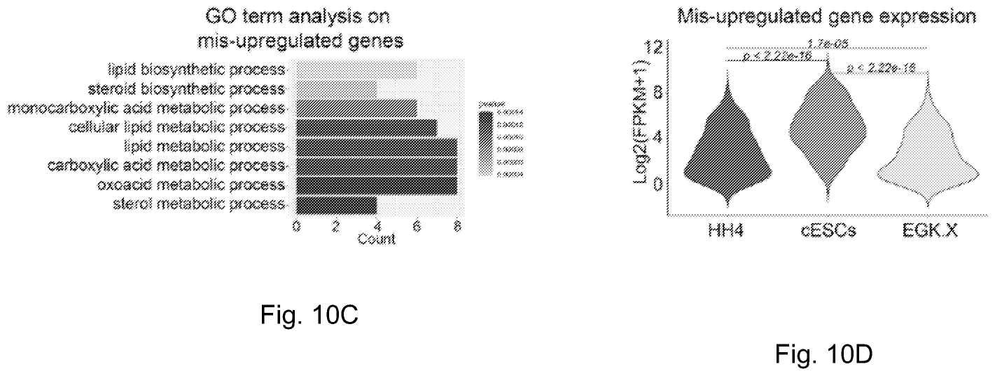

To further characterize the cellular identity, we performed RNA-seq analysis of OT/3i/chLIF chicken ESCs. Unbiased principal component analysis of bulk RNA-seq data from chicken ESCs, compared to cells at EGK.VIII, EGK.X and HH4 stages showed that chicken ESCs are in close relatedness to EGK.X stage blastodermal cells from which chicken ESCs were derived (FIG. 4A, based on PC1). However, differences reside between ESCs and EGK.X cells which separate the two groups on PC2. As an additional test, we identified the signature genes from EGK.I to HH4 stages, heatmap analysis showed that ESCs and EGK.X cells were grouped together and were apart from cells at other developmental stages (FIG. 10A).

Next, we performed a detailed transcriptomic analysis of chicken ESCs and their in vivo counterpart, EGK.X cells together with HH4 cells at gastrulation stage. At the whole transcriptome level, chicken ESCs present a higher linear correlation with EGK.X cells (R=0.8) versus HH4 cells (R=0.74) (FIG. 10B). Gene set enrichment analysis also confirmed that EGK.X, not HH4 signature genes, were highly expressed in chicken ESCs (FIG. 4B). Based on comparison with EGK.X and HH4 embryos, we categorized gene expression in chicken ESCs into three groups as either “successfully regulated”, “mis-regulated” or “failed” (FIG. 4C). Majority of the genes in chicken ESCs belongs to the successfully-regulated group, meaning that these genes were either upregulated or downregulated in both chicken ESCs and EGK.X stage embryos relative to HH4 stage embryos. Many well-known pluripotent genes such as Nanog, Pou5f3 and Klf4 were listed in this group and expressed highly in chicken ESCs (FIG. 4D). Gene Ontology (GO) term enrichment analysis of genes in the successfully-downregulated group revealed many differentiation/development related GO terms, suggesting that OT/3i/chLIF condition effectively blocked ESC differentiation (FIGS. 4E and 4F). In another aspect, 384 genes that have no statistical difference between EGK.X and HH4 stage embryos were inappropriately upregulated in chicken ESCs. GO term analysis showed that these genes were mainly involved in metabolic processes, which could be largely explained by the environmental changes between in vitro and in vivo conditions (FIGS. 10C and 10D).

In the mouse, at least three pluripotent states (naïve, formative, and primed) have been proposed. To access the pluripotent state of chicken ESCs, we performed transcriptome correlation analysis and found that chicken ESCs are more closely related to mouse formative pluripotent stem cells than mouse naïve and primed pluripotent stem cells (FIG. 4G).

Collectively, our results demonstrate that OT/3i/chLIF can capture and retain chicken ESCs in a pluripotent state similar to in vivo chicken EGK.X stage blastodermal cells.

### Chicken ESCs can Efficiently Contribute to Chimera Formation

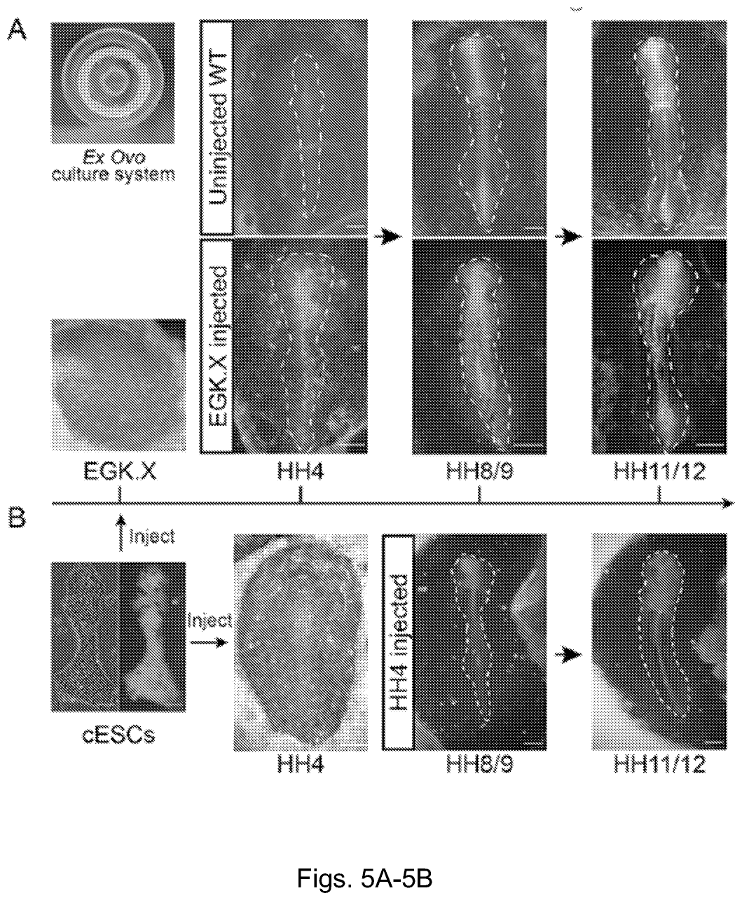

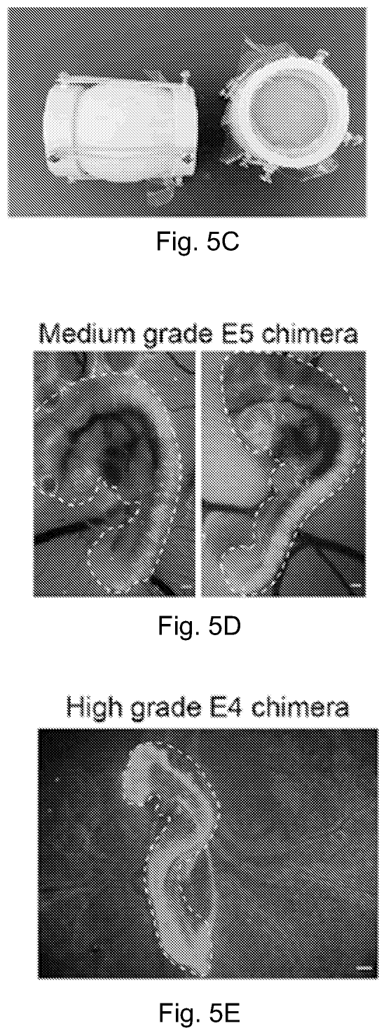

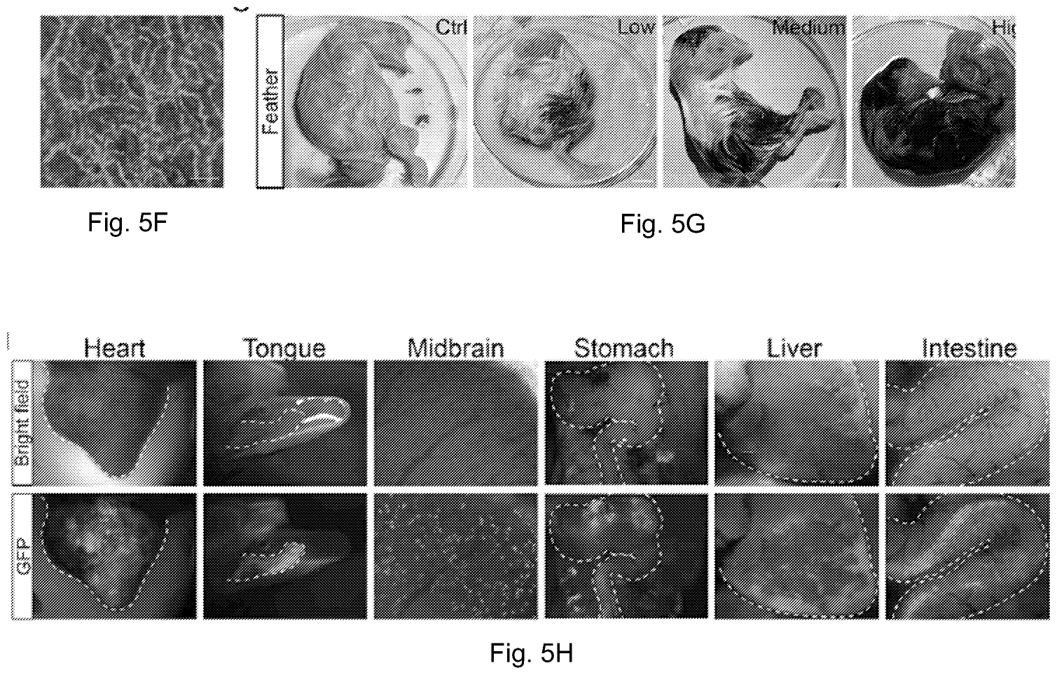

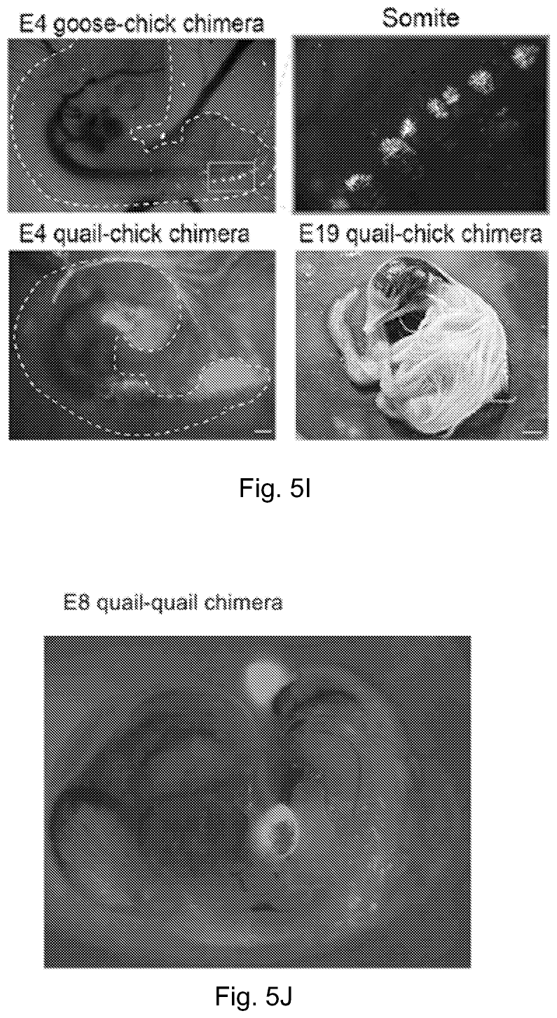

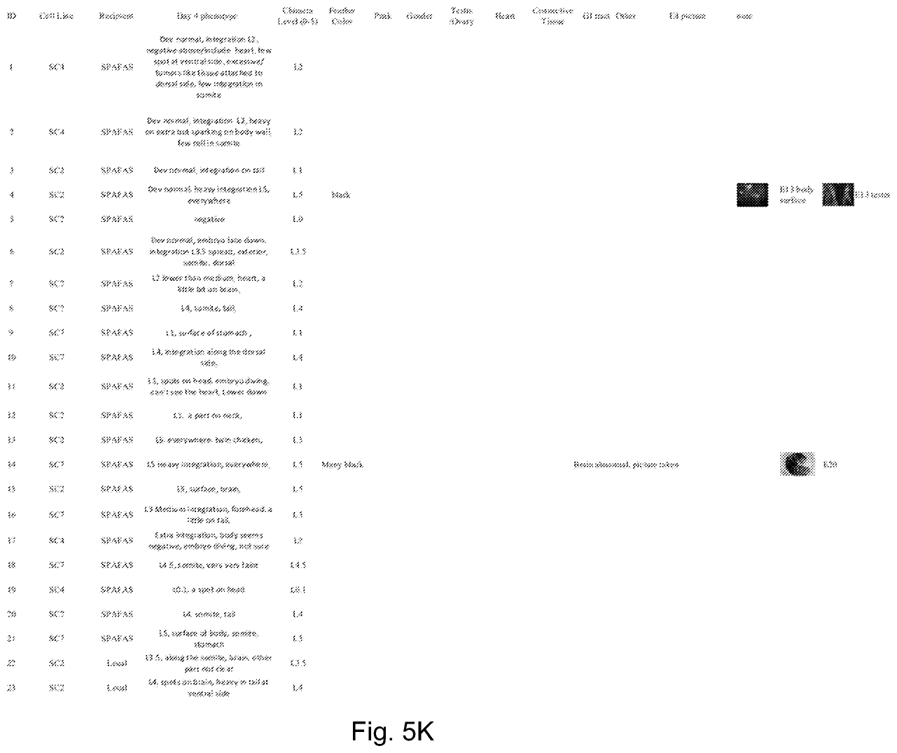

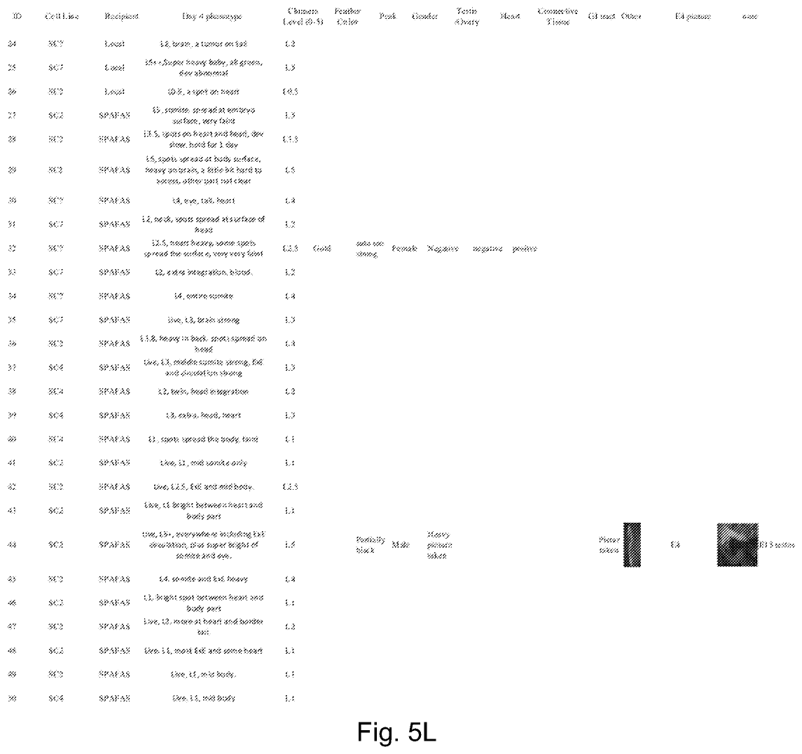

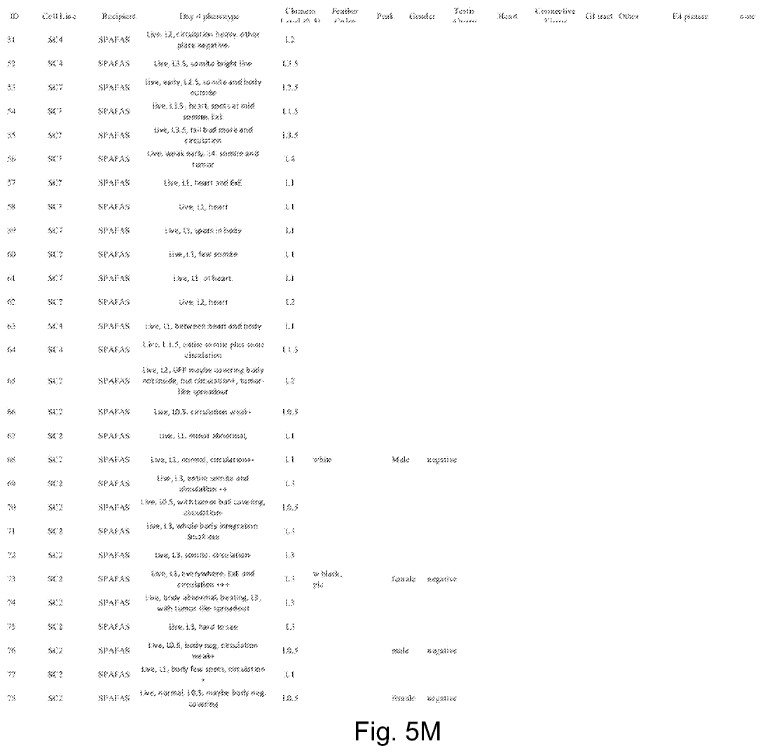

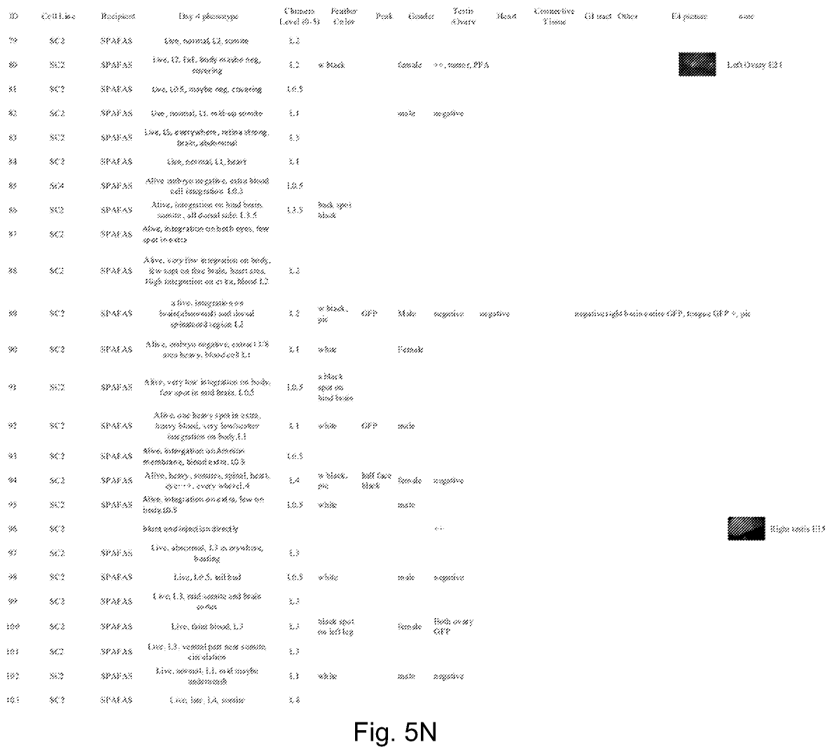

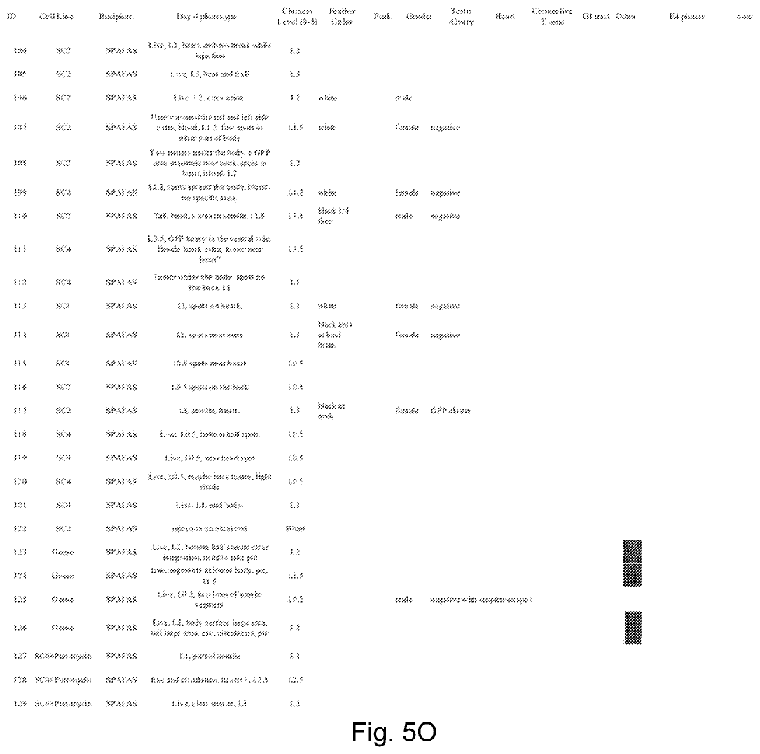

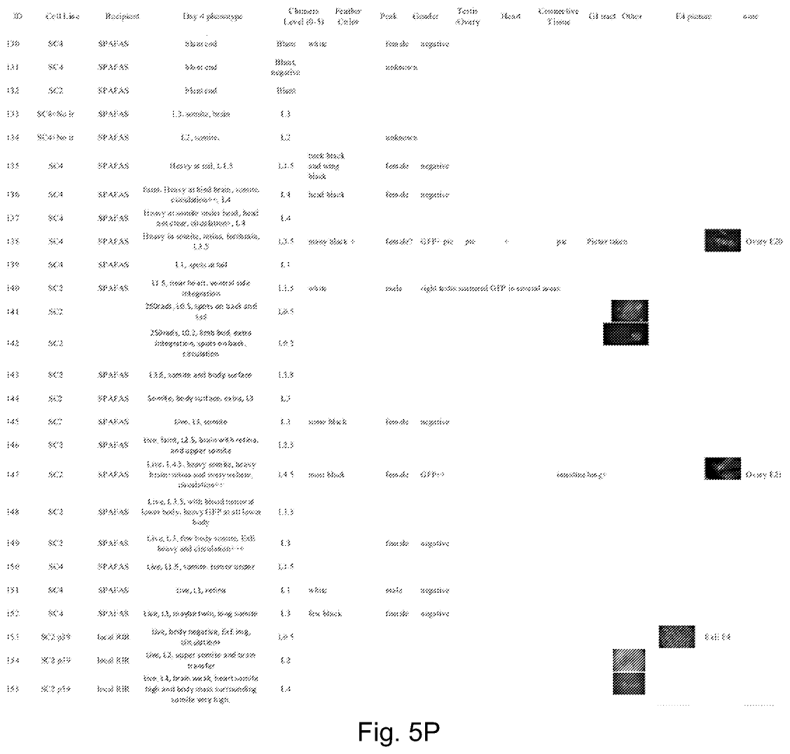

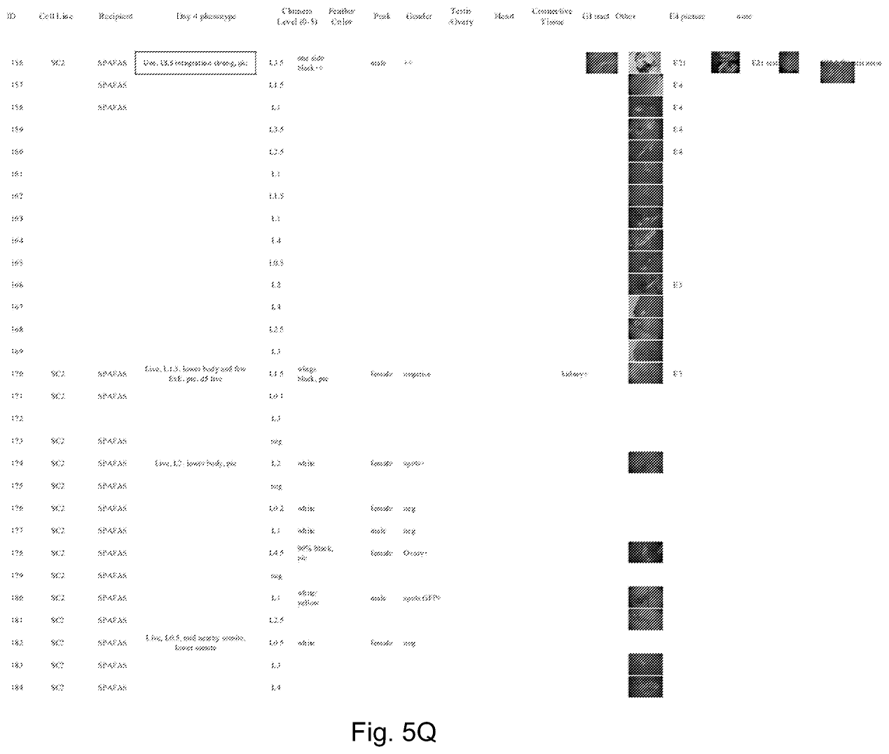

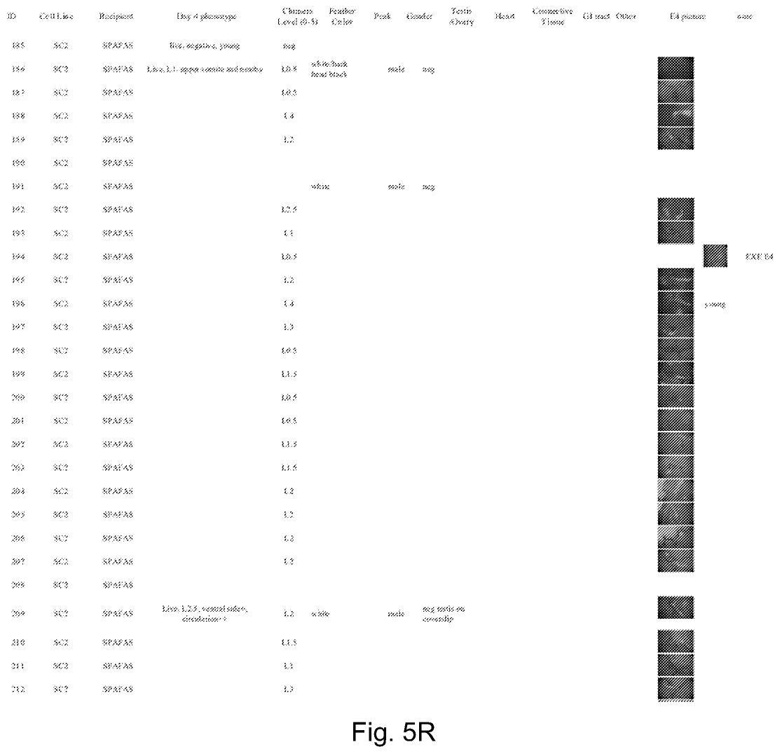

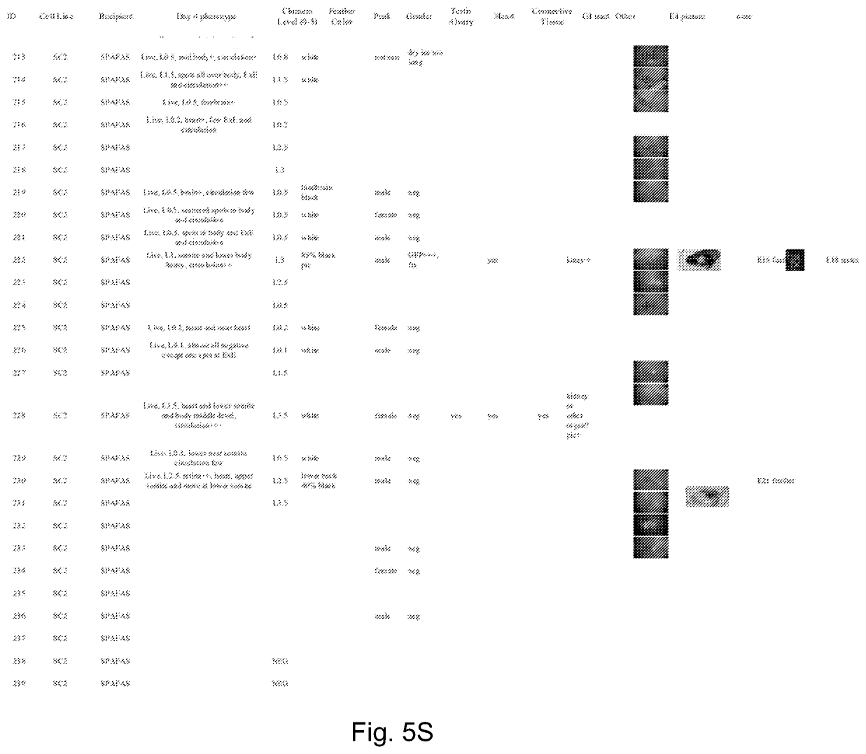

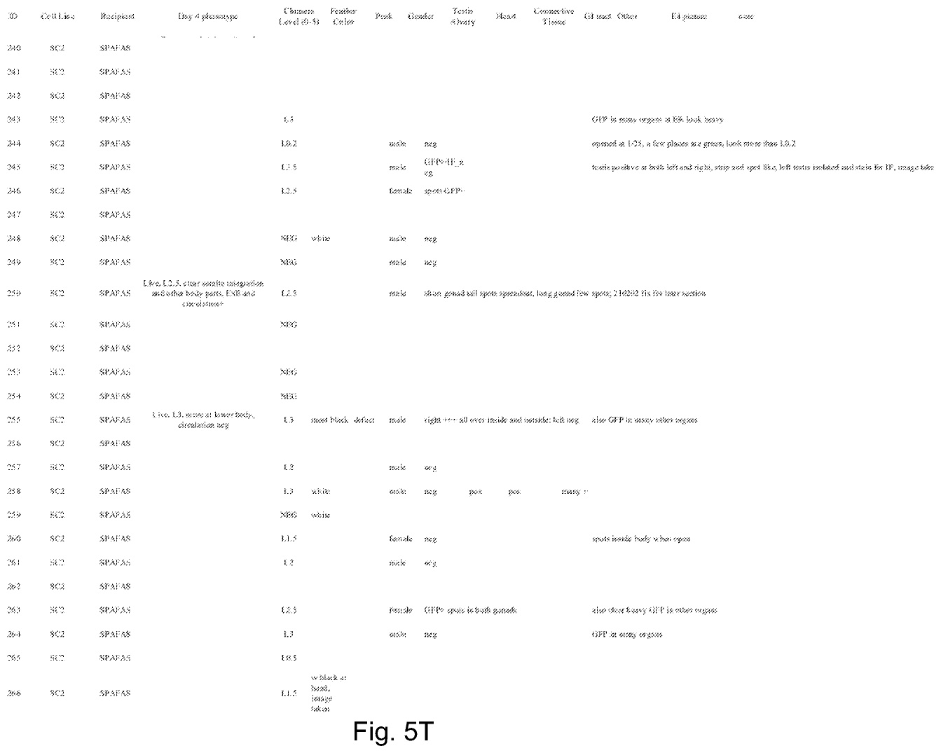

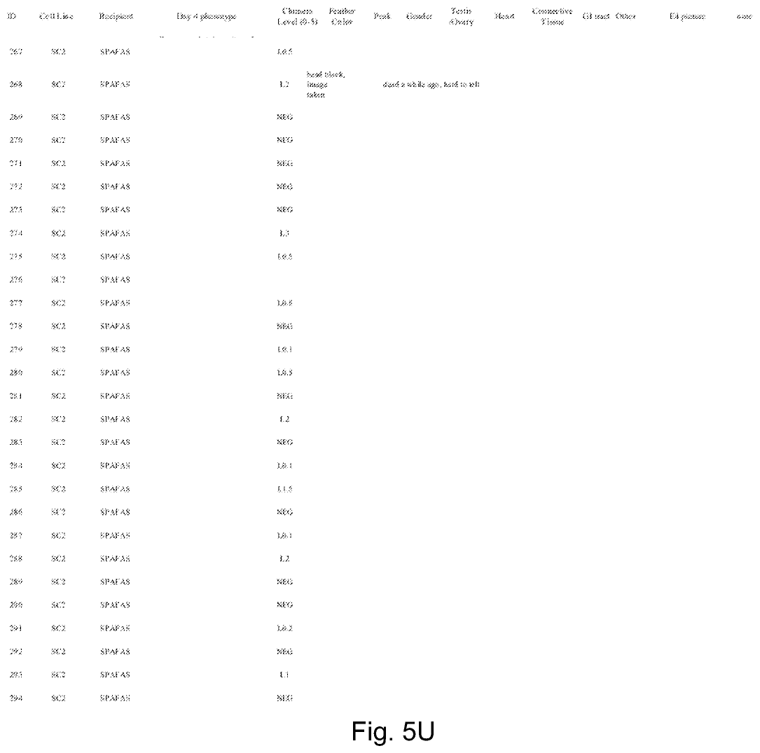

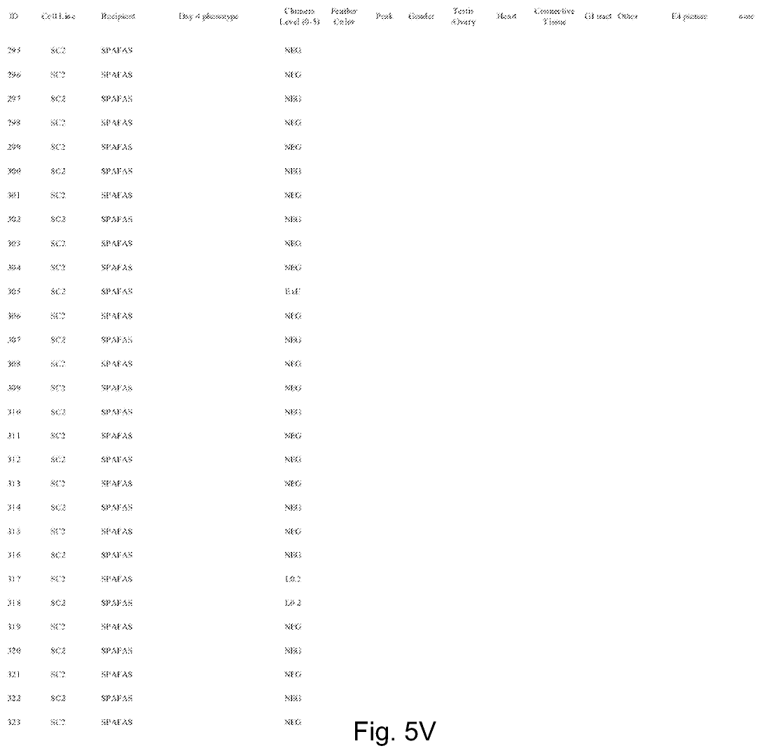

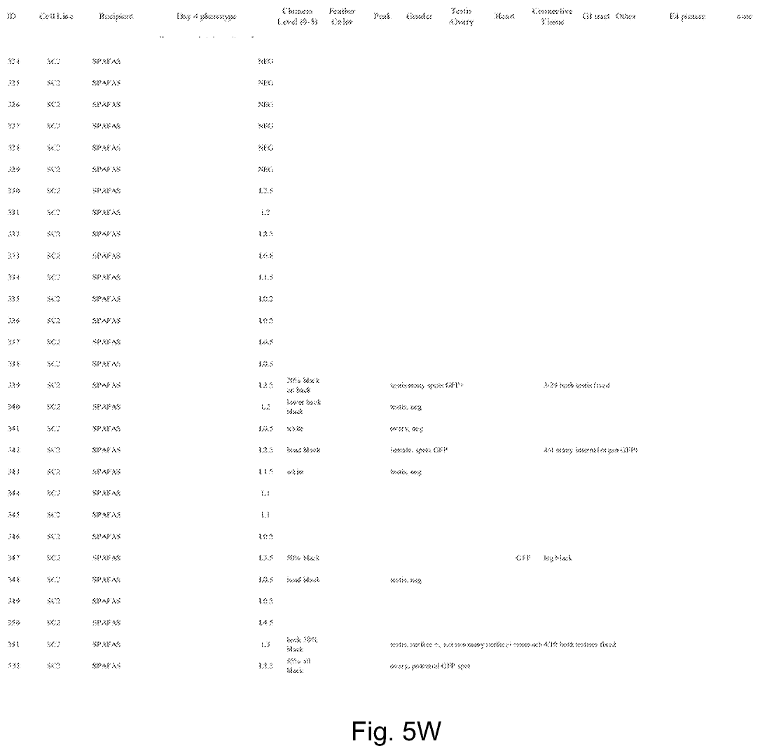

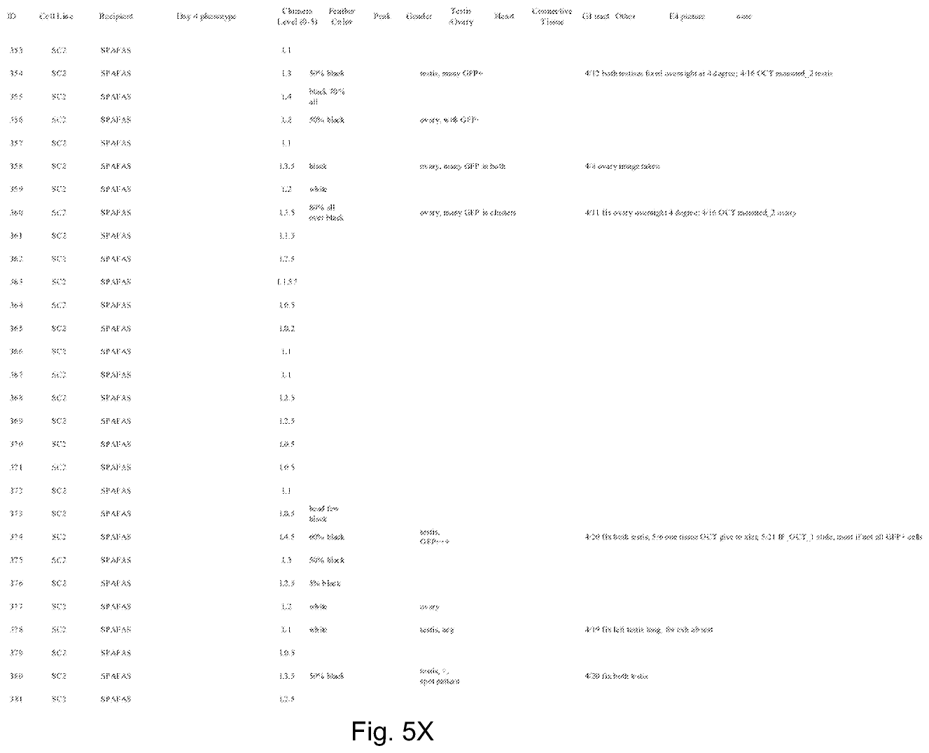

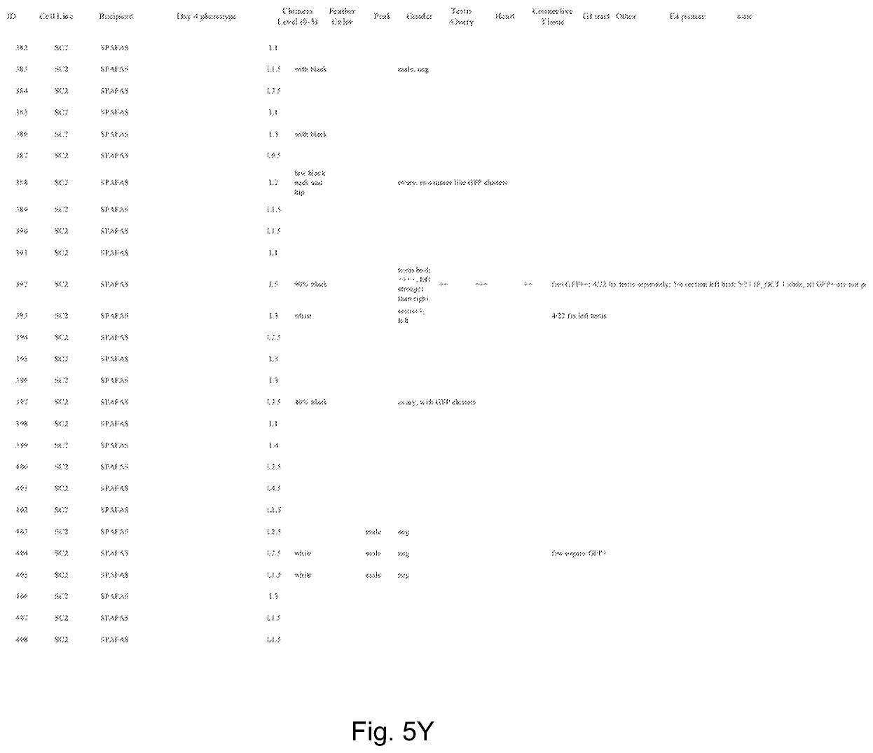

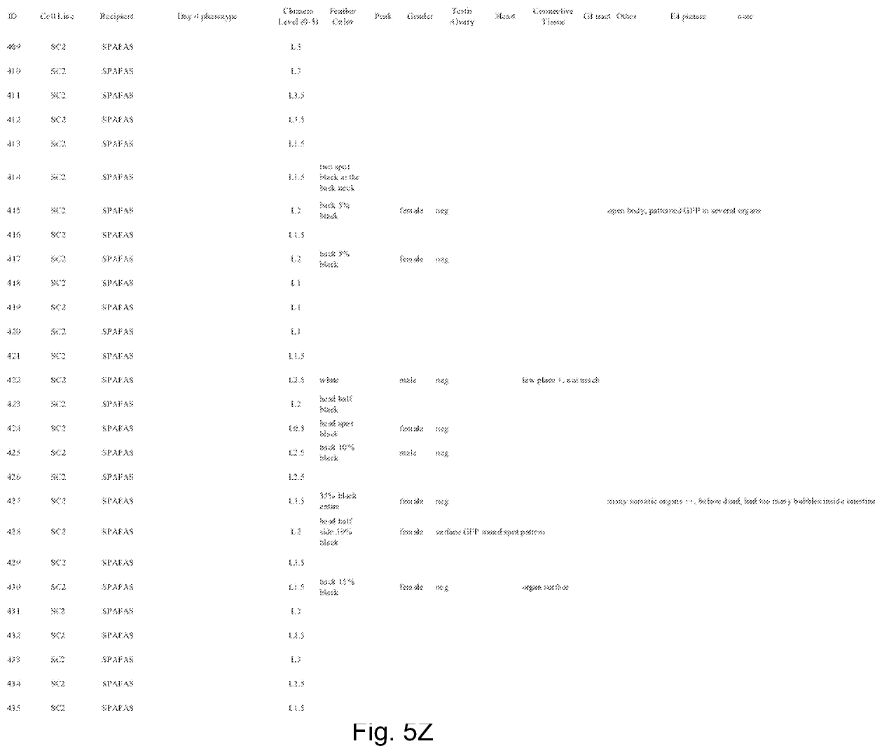

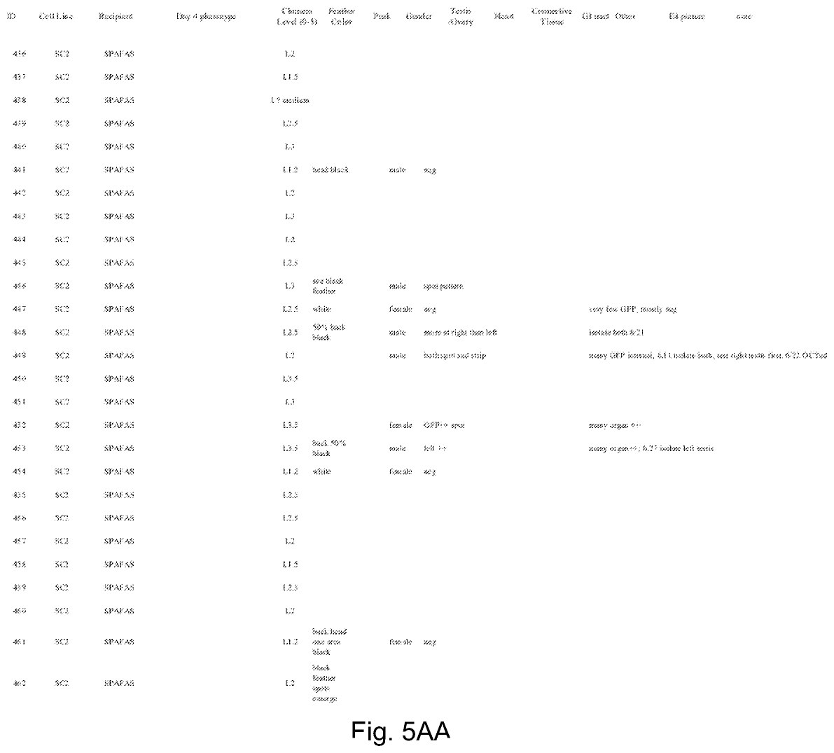

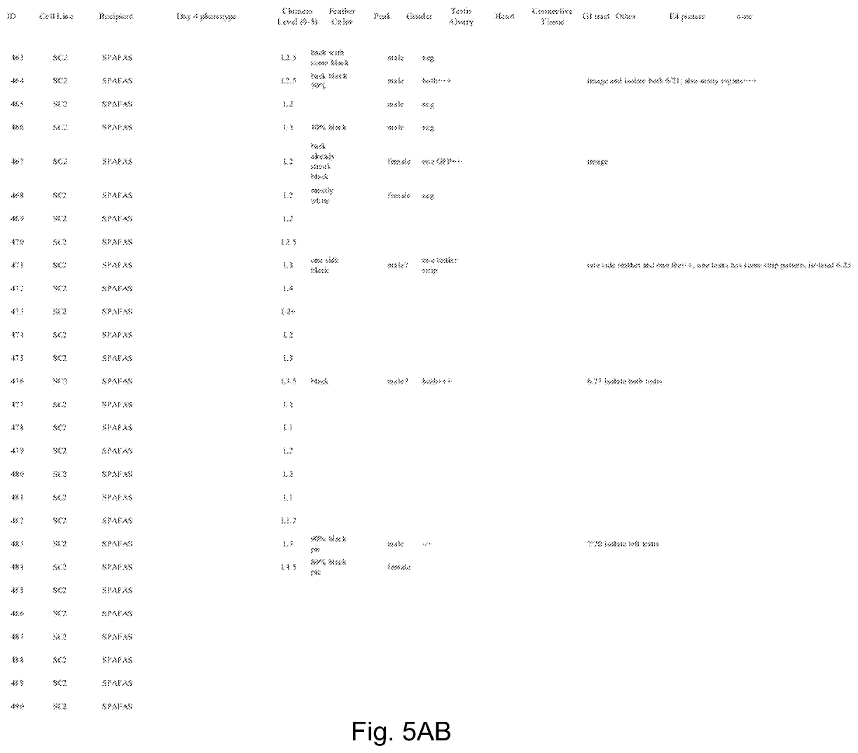

The ability to contribute to chimera formation is the defined feature of authentic ESCs. To test whether OT/3i/chLIF chicken ESCs could contribute to chimera formation, we implanted them into ex ovo incubated chicken embryos (FIG. 5A). Chicken embryos at EGK.X stage can develop normally ex ovo for up to 4 days. When implanted into EGK. X stage embryos, GFP-positive chicken ESCs efficiently integrated into the developing chicken embryos to form chimeric embryos (FIG. 5A and Table 3 in FIG. 5K). In contrast, chicken ESCs failed to integrate when implanted into HH4 stage embryos (FIG. 5B and Table 3), highlighting the importance of matching developmental stages between donor cells and recipient embryos.

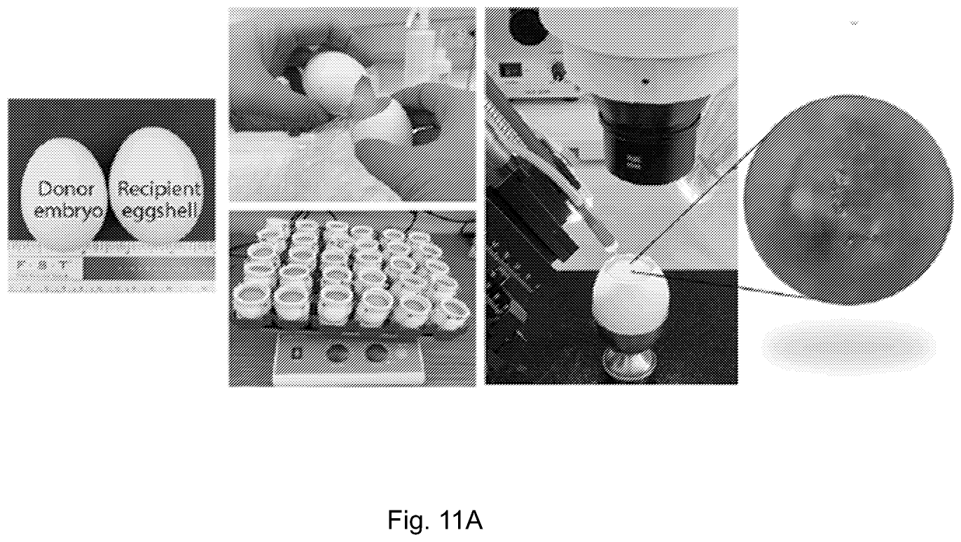

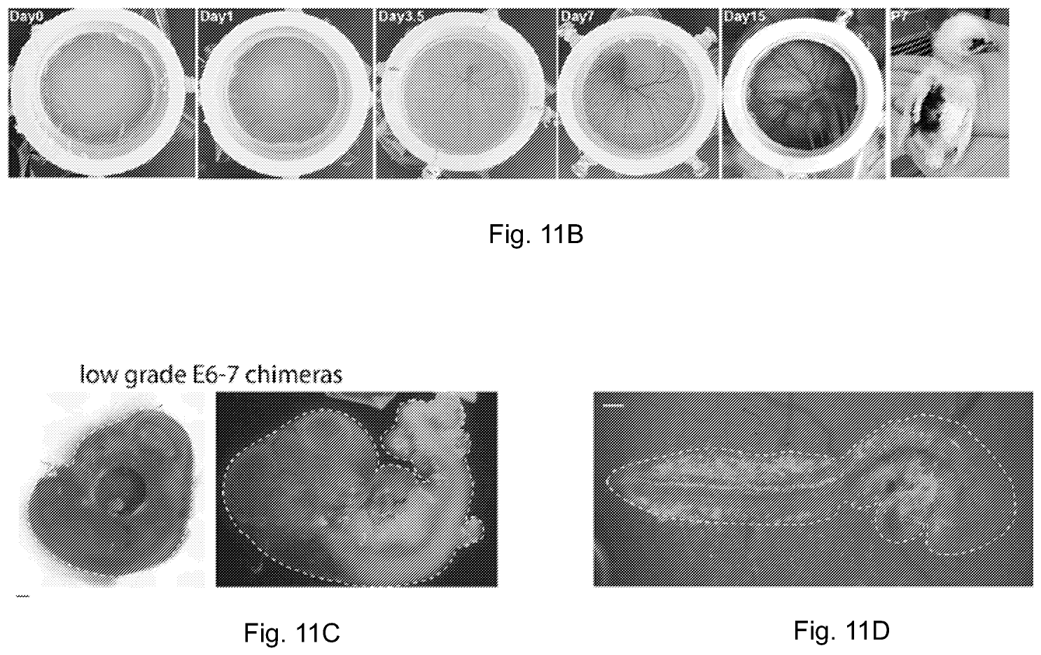

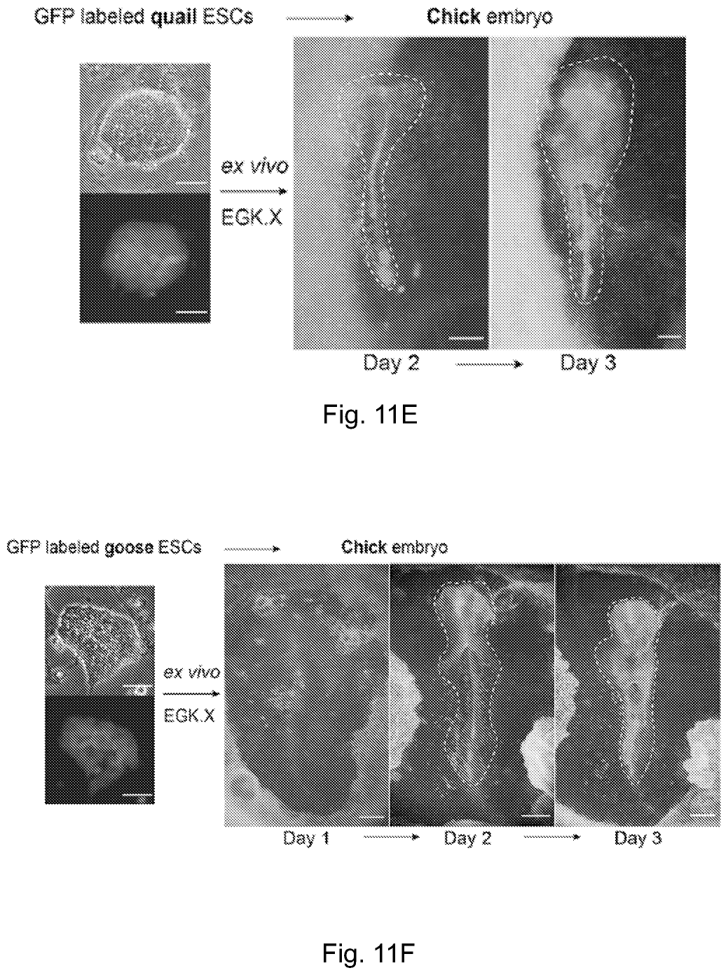

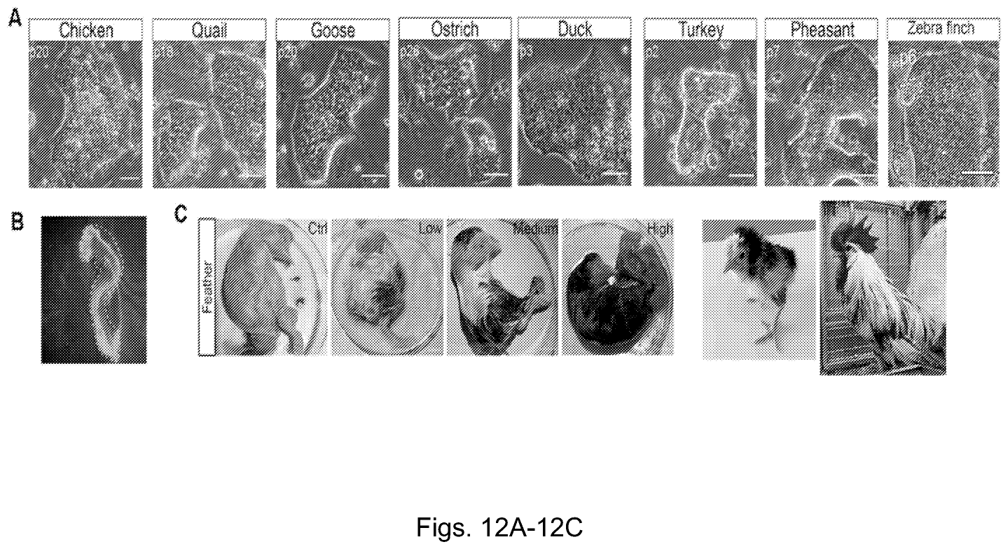

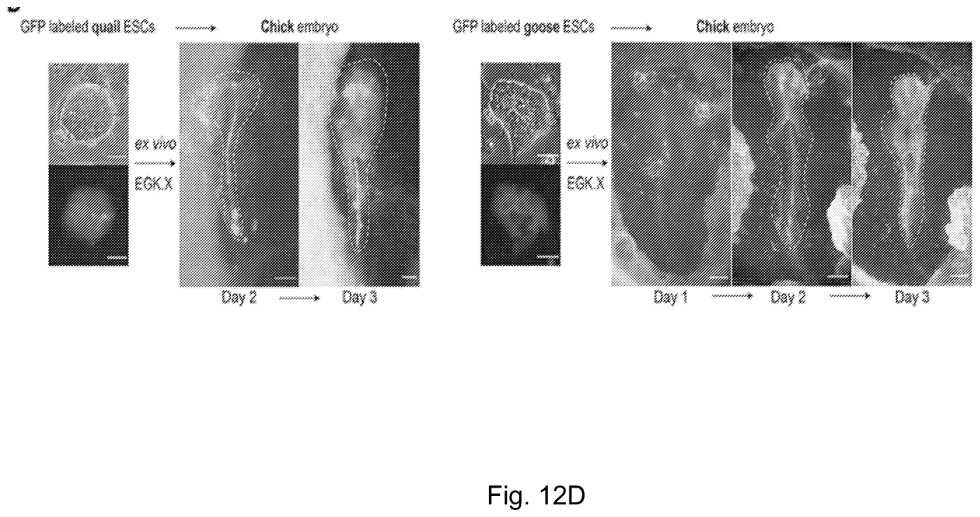

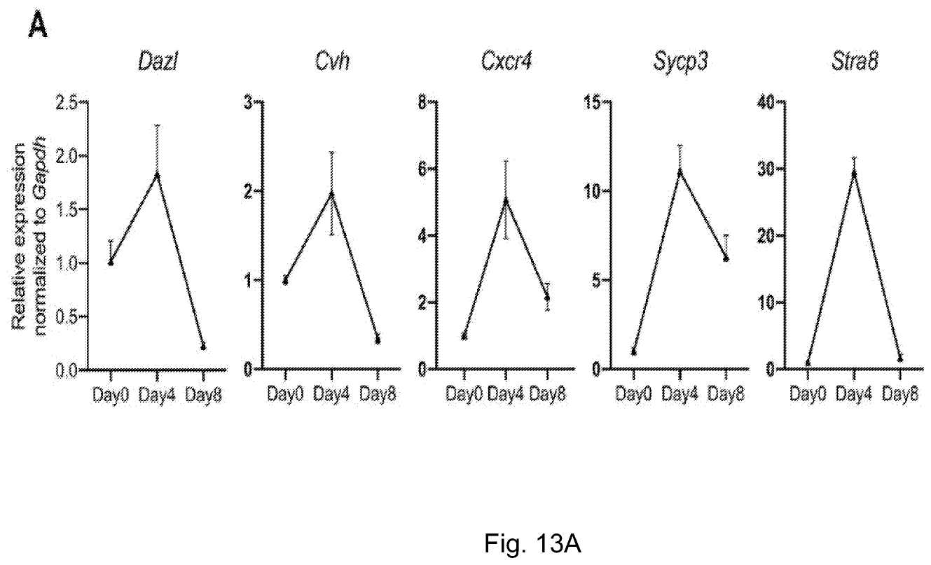

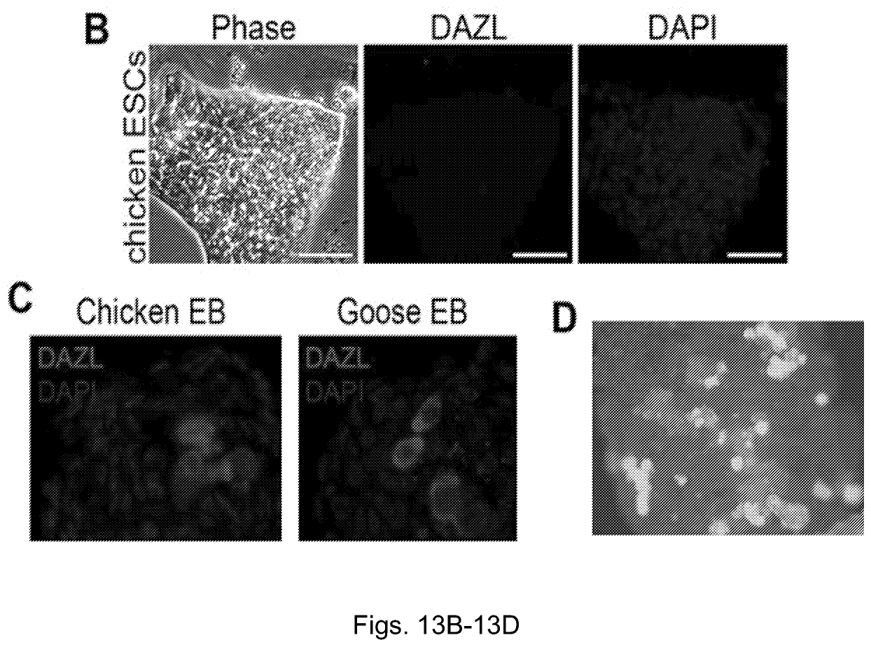

While the ex-ovo culture system provides a straightforward platform to assess the chimera-formation capacity of chicken ESCs, the use of the in ovo system is necessary to monitor the development of donor cells over long-term. We adopted the surrogate open-shell system to monitor chimeric embryo development until hatchling (FIGS. 5C and 11A). We injected GFP-positive chicken ESCs into the subgerminal cavity of EGK.X stage chicken embryos and monitored embryonic development and the distribution of GFP-positive cells through the window sealed with plastic film (FIG. 111B). Chicken ESCs contributed to multiple lineages inside the chicken embryos in ovo (FIG. 11C). Nonetheless, the overall level of in ovo chimerism was low compared to that of ex ovo chimeras.

Sublethal irradiation of the recipient chick embryos can increase the contribution of injected blastodermal cells. We applied this method and found that irradiation of the recipient embryos at 500-550 cGy prior to chicken ESC injection significantly increased the extent of chimerism without markedly compromising embryo viability (FIGS. 5D and 5E).

Long-term cultured chicken ESCs (passage 39) retained chimera-formation capacity (FIG. 1 ID). Notably, chicken ESCs gave rise to both somatic and extraembryonic cells in the chimeric embryos (FIGS. 4E and 4F), a feature that is different from mouse ESCs. To further assess whether functional engraftment sustained over time, chimeric embryos at later developmental stages (embryonic day 8-21) were analyzed. Overt coat color chimeras were obtained, corresponding to their chimera rate observed based on GFP coverage at E4 (FIG. 5G). Donor cell contribution into organs at multiple germ layers were also detected (FIG. 5H). A summary of chimeric embryos generated is shown in Table 3 of FIG. 5K. Collectively, the ex ovo and in ovo experiments demonstrated that chicken ESCs can efficiently integrate into embryonic development and contribute to both somatic and extraembryonic cell lineages.

### Quail and Goose ESCs can Contribute to Chimera Formation

In order to test the developmental potential of ESCs established from other avian species, we generated quail and goose ESCs with constitutively GFP expression (FIGS. 11E and 11F). Since quail-chick chimera can be produced by injecting quail blastodermal cells into chick embryos, we reasoned this interspecies chimera system could be used to test the chimera competency of avian ESCs. We implanted GFP-positive quail ESCs and goose ESCs onto ex ovo incubated EGK.X stage chick embryos. Donor cells were able to integrate into recipient chick embryos (FIGS. 11E and 11F). To further verify the chimera formation capacity of avian ESCs, we injected GFP-positive quail and goose ESCs into chick embryos in ovo and the result showed that quail and goose ESCs could contribute to chimera formation inside developing chick embryos (FIG. 4I). Additionally, extensive integration can also be achieved by injecting quail ESCs into the subgerminal cavity of quail embryos in ovo (FIG. 4J). These results demonstrate that quail and goose ESCs can also efficiently contribute to chimera formation.

Avian ESCs can Differentiate into Germ Cells Both In Vitro and in Ovo

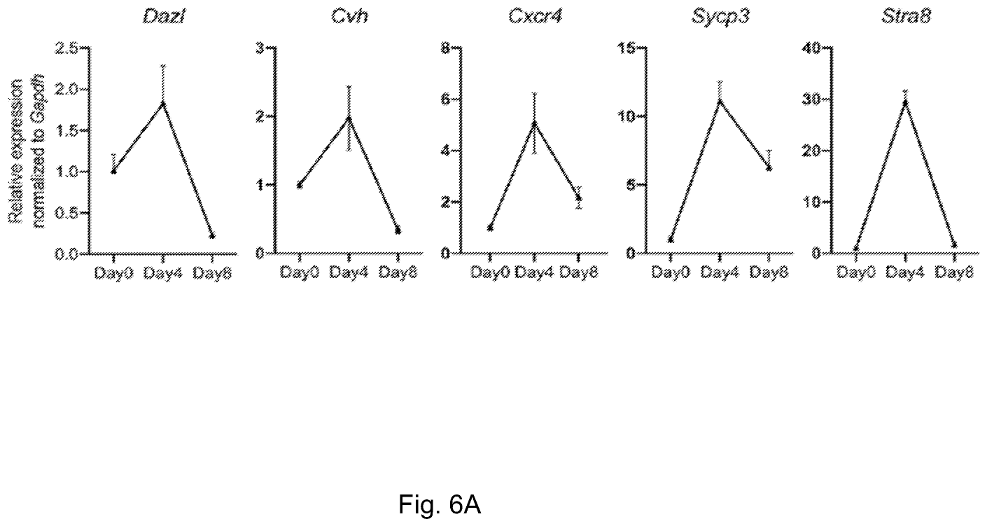

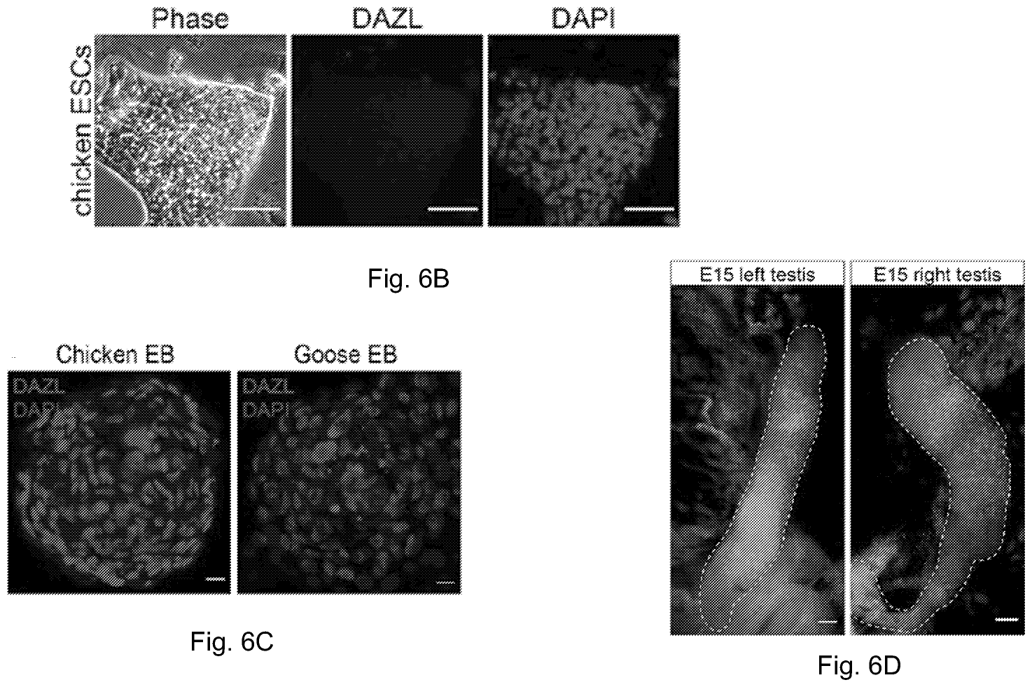

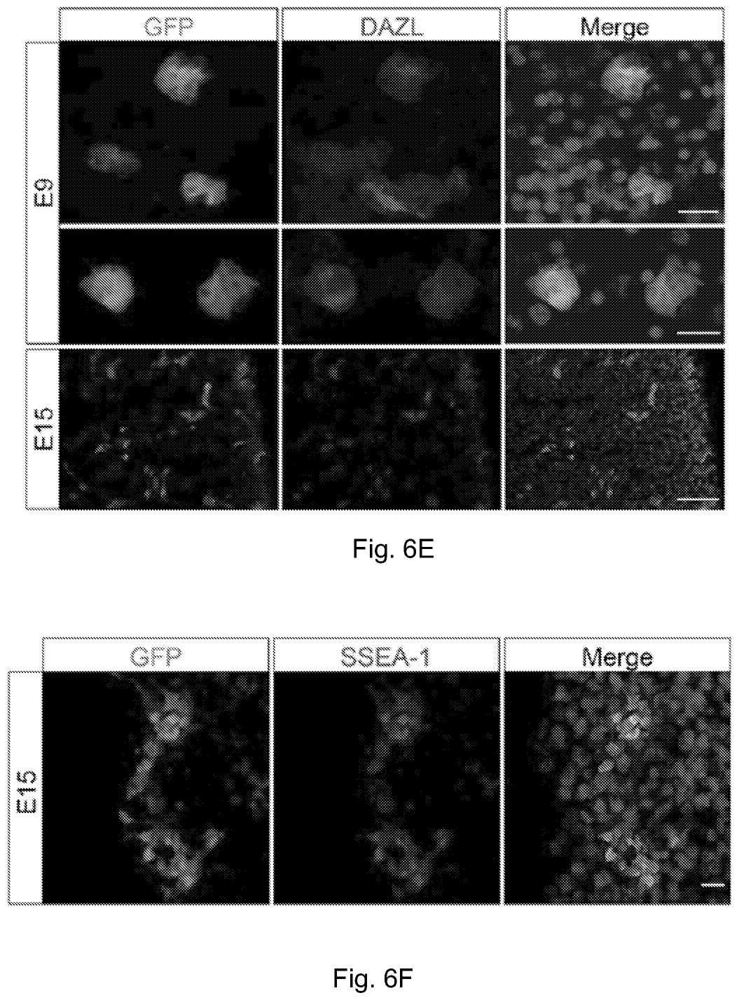

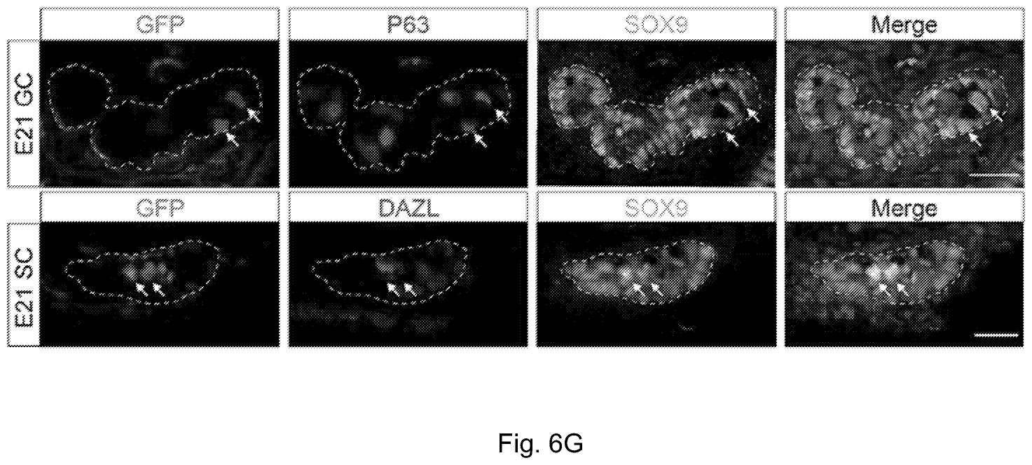

Next, we assessed the potential of chicken ESCs to differentiate into the germ cell lineage. We generated EBs from chicken ESCs and examined the expression of germ cell markers including Dazl, Cvh, Cxcr4, Sycp3 and Stra8. The relative expression levels of these germ cell markers significantly increased in day-4 EBs compared to undifferentiated chicken ESCs, but subsequently decreased in day-8 EBs (FIG. 6A), probably because the germ cell population was diluted by the rapidly expanding somatic cells within the EBs. To further investigate germ cell differentiation potential of chicken ESCs, we examined the expression of key primordial germ cell (PGC) marker, DAZL by immunofluorescence staining in both undifferentiated chicken ESCs and differentiated EBs. As expected, we did not detect DAZL expression in chicken ESCs (FIG. 6B). A subset of cells (1-3%) in day 3-4 EBs stained positive for DAZL (FIG. 6C). DAZL expression could also be detected inside goose ESC-derived EBs (FIG. 6C). Notably, the efficiency of DAZL expression varied among different ESC lines as no DAZL-positive cells could be detected in EBs derived from 11 out of 13 chicken ESC lines tested. Additionally, avian ESCs did not respond to BMP4 for the induction of PGCs, as mammalian ESCs did (data not shown).

To examine germline contribution in ovo, we injected male GFP-positive chicken ESCs into EGK.X stage chicken embryos pretreated with 500-550 cGy irradiation. Sublethal irradiation can increase the germline transmission rate of donor PGCs in chicken by reducing the number of endogenous PGCs. GFP-positive donor cells could be detected inside the gonads of chicken embryos injected with chicken ESCs, especially in high-grade chimeras (FIG. 6D). We analyzed the presence of ESC-derived germ cells in chimeric embryos at different developmental stages. In embryonic day 9 (E9) chimeric embryos, DAZL-positive GFP cells were localized in gonads as small clusters consisting of 1-3 cells (FIG. 6E). In E15 chimeric embryos, as PGCs proliferated, bigger clusters of DAZL-positive GFP cells could be found in gonads and the identity of these donor PGCs was further confirmed by staining with SSEA-1 (FIGS. 6E and 6F), another germ cell marker. In E21 embryos, as gonads continued to develop, PGCs were enclosed inside the seminiferous tubules. GFP-positive PGCs could be detected within the tubules together with endogenous PGCs by staining with P63, a marker for germ cells. Additionally, in some seminiferous tubules, GFP cells also gave rise to Sertoli cells shown by SOX9 (FIG. 6G). Notably, the contribution of ESCs into germline lineage inside chicken embryos was confirmed using multiple cell lines derived (Table S3). Importantly, cell lines failed to express DAZL in vitro were able to give rise to germ cells after transplanting into chicken embryos, suggesting a more germline-favored environment in ovo in comparison to the spontaneous differentiation environment in vitro. Germ line contribution in different levels of chimeric embryos was summarized in Table S3. In some high-grade chimeras, almost all of the PGCs observed were originated from donor ESCs (FIGS. 6E and 6F). Taken together, through tracing of the germline development of donor avian ESCs, we demonstrated that avian ESCs are competent to adopt a germ cell fate both in vitro and in ovo.

### Chicken ESCs Contribute to Feather Patterning in Adult Chimeric Chicken

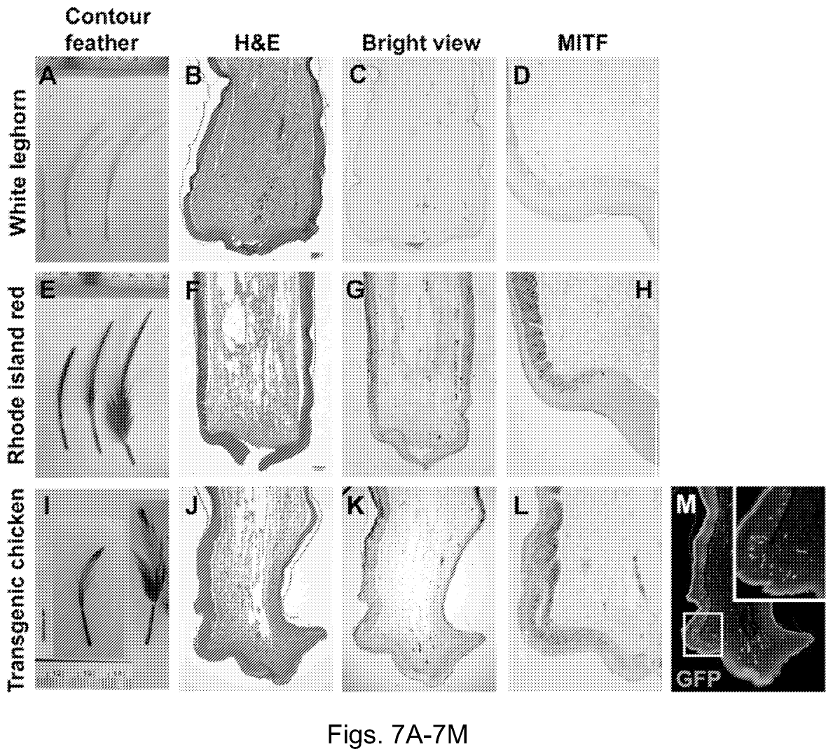

The transgenic chickens displayed pigmented feathers. However, the recipient chicken strain is White leghorn which is apigmented. White leghorn chicken lost the entire melanocyte lineage in the feather. To examine whether ESCs from Rhode Island red chicken contribute to the melanocyte progenitor in white leghorn chicken and further make it pigmented, we collected the growth phase contour feather follicles from adult chimeric chicken and compared to the feathers from adult white leghorn chicken and Rhode Island red chicken (FIGS. 7A, 7E, and 7I). Paraffin sections were prepared, and H&E staining showed the normal development of feather follicle in chimeric chicken (FIG. 7I, compared to FIGS. 7B and 7F). Bright view of feather follicle showed the pigmented transgenic feather (FIG. 7K, compared to apigmented White leghorn feather chicken in FIG. 7C, and pigmented Rhode Island red chicken feather in FIG. 7G). Staining of MITF, a marker for melanocyte progenitor cells, shows the transgenic chicken feather expressing MITF (FIG. 7L), whereas White leghorn chicken feather is MITF negative (FIG. 7D). These transgenic chicken feathers are also GFP positive (FIG. 7M). These results suggest that ESCs from Rhode Island red chicken contribute to the melanocyte progenitor cells generation and make the feathers in chimeric chicken pigmented.

### Discussion

In this disclosure, we report, for the first time, derivation of germline competent embryonic stem cell lines from avian species. Avian ESCs could be efficiently derived and maintained long-term without losing their pluripotent potential. Furthermore, these avian ESCs comprehensively contribute to chimera formation when reintroduced back to embryos. We also demonstrated that chicken ESCs can give rise to germ cells in ovo. The establishment of avian ESCs in combination with the advancing gene-targeting technology serve as a great platform for the generation of genetically modified avian species, providing exciting opportunities in developmental biology, bioreactor industry, poultry and many other related fields. Additionally, the development of avian ESC self-renewal condition plus the cellular reprogramming technology opens up the possibility to conserve endangered species and revive extinct avian animals.

In contrary to mammals, mode of PGC formation in birds remains controversial. It has long been thought that chicken adopts a maternal specification mode of PGC formation due to the detection of Ddx4 expression in early cleavage stages. Our finding that in-vitro expanded chicken ESCs could efficiently differentiate into PGCs challenges this idea. Analysis over various developmental stages validates the identity of chicken ESC-derived PGCs. Donor-derived PGCs follow the same developmental trajectory as endogenous PGCs including migration to the genital ridge, proliferation as clusters and being enclosed inside the seminiferous tubules at later stage. Notably, established avian ESC lines show variable in-vitro differentiation capacity into germline lineage. However, those cell lines could efficiently adopt a germline fate upon transplantation in ovo. We showed that avian ESC does not respond to mammalian induction cues for germline differentiation. Understanding the molecular mechanisms governing the differentiation of ESCs to PGCs will guide the generation of ESC-derived PGCs in vitro which would facilitate the generation of transgenic avian species.

The capture of ESCs across avian species is unprecedented, suggesting Wnt and PKC signaling play conserved roles governing the pluripotency of avian blastodermal cells. However, simply applying the chicken ESC self-renewal condition (OT/2i) does not work for other avian species because subtle difference between closely related species shifts the balance for cell fate control. Fortunately, a biased differentiation potential towards beating cardiomyocytes guide us to tweak our culture condition, leading to the discovery of the self-renewal-promoting effect of SB431542 and chicken LIF. Interestingly, these two compounds have been tested and excluded from our initial hits during the development of chicken ESC culture condition due to their obscure function for chicken ESC self-renewal. After discovering their roles in other avian species, we re-examined them in chicken ESCs and were able to reveal their function to stabilize and proliferate chicken ESCs in long-term passages. We also noticed that the positive role for SB431542 largely relies on the preconditioning of avian ESCs with Gö6983 and IWR-1 since SB431542-alone does not promote the self-renewal of avian ESCs. This result suggests a role of SB431542 downstream of Gö6983 and IWR-1 to balance the shift caused by the two primary compounds. Such readjustment is dispensable for chicken ESC derivation but is critical for the derivation of other avian ESC lines. The differentiating effect of LIF on ostrich ESCs is worth noting and contrary to the self-renewal-promoting effect of LIF on most of the other avian ESCs we tested. This finding suggests that difference on signaling dependence exists even among closely related species. In summary, the discovery of a conserved pluripotent signaling across avian species emphasizes the benefits of optimizing culture condition using closely related species and acknowledges the value for supportive compounds to calibrate the balance shift caused by the primary compounds. We mainly focused on chicken, quail and goose ESCs in this project. Comprehensive investigation is required to examine the developmental potential of other avian ESCs through chimera formation and germline transmission assay.

The development of avian ESC culture condition shares many lessons for the derivation of pluripotent cell lines from other species. Most of the effective small molecules and cytokines we used here are not unique to birds and have been reported in studies on ESCs from mouse and other species as well. This feature implies that key signaling pathways regulating pluripotency are well conserved across species. What makes it challenging is to find out the exact drug combination which requires an understanding of the pluripotent state of primary epiblast cells. Looking back to early chick development, we noticed that antagonists of Wnt and Nodal pathways naturally present in chick embryo to block ectopic primitive streak formation. Therefore, understanding the signaling responses of primary cells will guide the derivation of stem cell lines in vitro. Another important lesson comes from yolk supplement and the identification of ovotransferrin from it. Most of the basal culture medium is designed for mammalian cell growth and essential components for cell survival could be missing for non-mammalian cells. Although we could find out the cell-proliferating function of yolk supplement for chicken ESCs, yolk supplement promoted the differentiation of ESCs from other avian species. Identification of ovotransferrin excludes the negative fractions from yolk which opens up the opportunity to optimize our culture condition for avian ESC growth. We anticipate similar scenarios could happen for derivation of stem cells from other species and missing components for cell survival are critical for successful derivation.

Works on mouse pluripotent stem cells have defined pluripotency into naïve and primed state, and more recently a formative phase which lies between them. It will be interesting to compare our avian ESCs with mouse pluripotent stem cells in different states. Naïve state refers to inner cell mass at preimplantation mouse embryo and naïve mouse ESC can be propagated in dual inhibition of MEK and GSK3. Through our initial screening, we know that neither MEK nor GSK3 inhibition supports the self-renewal of avian ESCs. Interestingly, transcriptome comparison of avian and mouse pluripotent stem cells revealed that chicken ESCs are in closer correlation with mouse naïve and formative ESCs, not primed EpiSCs. This result is somewhat unexpected because epiblasts at EGK.X-XII stage have already shown regional bias in cell fate commitment prior to gastrulation. This biased fate commitment at EGK.X stage also potentially explains the variation of donor cell lineage contribution in chimeric embryos. Although correlated in transcriptomic profile, avian ESCs still have many clear differences from mouse ESCs such as the distinct response to Wnt and MAPK signaling. The recent notion of formative pluripotency draws our attention since avian ESCs have many similar characteristics as mouse cells in formative state. Nevertheless, detail investigation of transcriptome and epigenetic profiling is required to further define the pluripotent state of avian ESCs.

Chicken as a model system has a long and successfully history. However, the development of chicken system has been largely delayed due to the lack of bonajfde ESCs. Our derivation of germline-competent chicken ESCs fills this gap and provides exciting opportunities for future studies using the chick system. Additional derivation of ESCs from other avian species opens up new avenue as new modeling systems for biological research and also have unlimited great potential in poultry and biotechnology industries.

### Experimental Model and Subject Details

### Animal

All animal procedures were carried out in accordance with approved guidelines from the University of Southern California Institutional Animal Care and Use Committees.

### Avian ESC Derivation and Maintenance

All avian embryos were determined according to the Eyal-Giladi and Kochav staging system and the Hamburger and Hamilton staging system (Eyal-Giladi H, Kochav S. From cleavage to primitive streak formation: a complementary normal table and a new look at the first stages of the development of the chick. I. General morphology. Dev Biol. 1976 April; 49(2):321-37; Hamburger V, Hamilton HL. A series of normal stages in the development of the chick embryo. J Morphol. 1951; 88:49-92).

Freshly laid avian fertile eggs at EGK.X-XII stages were kept at 16° C. before extraction. To harvest cells from yolk, a filter paper-based method was applied. Filter paper with a hole at center was placed on top of the embryo. The outer edge of the filter paper was cut together with the vitelline membranes. Embryo were then released from yolk together with the filter paper and washed in PBS solution. Next, embryo was transferred into a 15 ml tube free of the paper ring and dissociated by pipetting inside N2B27 medium. Dissociated cells were cultured in tissue plates coated with irradiated mouse embryonic fibroblast (MEF) cells as feeder layer. Basal medium used for routine ESC maintenance was N2B27 medium composed of Dulbecco's Modified Eagle Medium/Nutrient Mixture F-12 (DMEM/F12):Neuralbasal (1:1) plus 0.5×N2, 0.5×B27 supplement and 20 μg/ml ovotransferrin with the addition of 1× penicillin-streptomycin. Avian ESC self-renewal was kept with 2.5 μM IWR-1, 2 μM Gö6983, 2 μM SB431542 plus chicken LIF (10 ng/ml for ESCs and 40 ng/ml for other avian ESCs). ESCs were cultured at 38° C. in 5% CO₂ with daily medium change. To passage, TrypLE was used to incubate the cells for 5 min and neutralized with basal medium in triple amount. Cells were handled with 5 ml pipette all the time to avoid dissociation into singles. Cells can be frozen in Cellbanker 2 and stored at −150° C.

### Method Details

### Generation of GFP-Labeled ESC Lines

Avian ESCs carrying constitutively expressed GFP were generated by lentivirus infection. Lenti-X 293T cells (Takara) were transfected with pSIN-EF2-GFP-Puro plasmid together with pSPAX2 and pVSVG via polyethylenimine (PEI 25,000, Polysciences). Lentivirus particles were then collected and concentrated by Lenti-X concentrator (Takara) according to manufacturer's instruction. After infected with lentivirus, avian ESCs were treated with 0.5 μg/ml puromycin for 24 hrs. This selection can be repeated for 3-4 times until no obvious cell death upon treatment and a relatively homogenous GFP expression can be further verified by fluorescence microscope. The sex of each GFP-labeled avian ESC line is determined by PCR using Z and W sex chromosome specific primers.

### RNAscope In Situ Hybridization

Detection of Nanog mRNA expression in chicken blastodermal cells was performed using RNAscope® 2.5 HD Assay kit-BROWN (Advance Cell Diagnostics) according to manufacturer's instruction. RNAscope probe targeting chicken Nanog mRNA (Gg-Nanog) was designed by Advance Cell Diagnostics. Chicken blastodermal cells were firstly isolated from yolk and then cultured in various conditions inside 4-well chamber slides coated with irradiated MEF cells (Nunc™ Lab-Tek™ II Chamber Slide™, Thermo Scientific). After 3-day culturing, cells were fixed in 4% paraformaldehyde/PBS for 30 min followed by dehydration and rehydration with EtOH. Cells were then pretreated with RNAscope hydrogen peroxide and protease plus before hybridization with chicken Nanog probe, Gg-PPIB as positive control and DapB as negative control for 2 hrs at 40° C. After a 6-step amplification process, cells were stained with DAB and counter-stained with 50% Hematoxylin staining solution. Slides were dehydrated, mounted and subjected to examine under microscope.

Quantitative Real-Time PCR (qRT-PCR)

Embryos were dissected from yolk using the filter paper method described above. Total RNA of chicken embryos and in-vitro expanded cells was extracted using the RNeasy Mini Kit (QIAGEN) according to manufacturer's instructions. cDNA was synthesized from 1 μg total RNA using the iScript™ Reverse Transcription Supermix (BIO-RAD) according to the manufacturer's instructions. qRT-PCR was performed using the iTag™ Universal SYBR® Green Supermix (BIO-RAD) on an Applied Biosystems 7900HT Real-Time PCR machine (Thermo Scientific). Gene expression was normalized to Gapdh expression.

### Egg Yolk Fractionation

20 ml of yolk was collected from 2 unfertilized chicken eggs and was further diluted with 80 ml 0.15 M NaCl (as 20% yolk). This 20% yolk was then centrifuged at 30,000 g for 2 hrs at 4° C. Yolk plasma was collected (as 20% plasma) and granule was resuspended in 50 ml 0.75M NaCl (as 20% granule). To further fractionate plasma, eight layers of paper towels (pre-soaked with ddH2O) was used to filter plasma and flow-through was then passed through 0.8 μm filter, followed by 0.45 μm and 0.2 μm filters sequentially (last flow-through product marked as 0.2 μm filtered). This 0.2 μm filtered flow-through was applied with Amicon® Ultra-15 Centrifugal Filter at 100 kDa and 50 kDa MWCO at 10,000 g for 1 hr at 4° C. sequentially to generate fractions marked as >100 kD, 50-100 kD and <50 kD. All the above fraction was considered as 20% in concentration and added into cell culture medium as a final concentration of 3% for testing.

Ammonium sulfate precipitation assay was performed to separate proteins from 50-100 kD fraction. Saturated ammonium sulfate solution was slowly added into the fraction to achieve 30% salt concentration and mixture was stirred at 4° C. for 1 hr followed by centrifugation at 5000 g for 30 min. Pellet was resuspended in 0.15 M NaCl and marked as 30p. The remaining supernatant was loaded again with saturated ammonium sulfate solution to achieve 40% salt concentration, followed by mixture and concentration. The same procedure was repeated to collect samples marked as 40p, 50p, 60p, 70p and 80p. All these fractions were centrifuged twice with Amicon® Ultra-0.5 Centrifugal Filter Unit at 3 KDa MWCO to remove any residual ammonium sulfate before cellular assay.

To reveal protein identity, ammonium sulfate fractions was separated by SDS-PAGE and stained with Coomassie blue. Gel fractions were cut and sent to Proteomics Core Facility at University of Southern California for LC-MS/MS analysis.

### In Vitro Differentiation of ESCs

Differentiation of ESCs into somatic and germline lineages were induced by the formation of EBs. Avian ESCs were dissociated by TrypLE and resuspended in N2B27 basal medium onto adherent culture plate for 1 hr to remove feeder cells. After that, loosely attached or floating avian ESCs were collected and plated onto Aggrewell™-400 24 well plate (Stemcell Technologies) at about 70,000 to 150,000 cells per well to allow EB formation in N2B27 basal medium. For germline and somatic lineage detection, EBs were harvested from Aggrewell after 4 days, followed by immunostaining and RT-PCR detection. For longer differentiation, EBs were transferred to non-adherent wells for an additional 4-day incubation before being harvested.

For differentiation into neural lineage, EBs were formed overnight using Aggrewell™-400 and then transferred to non-adherent wells for additional 3-day incubation in N2B27 medium plus 2.5 μM IWR-1, 2 μM Gö6983 and 2 μM SB431542. Medium was then replaced to N2B27 plus 5% chicken serum for another 7 days. Afterwards, EBs were plated onto Matrigel-coated 4-well plates and cultured for 2 more days before immunostaining.

For differentiation into cardiomyocytes, EBs were formed overnight using Aggrewell™-400 and then transferred to non-adherent wells for additional 3-day incubation in N2B27 medium plus 2 μM IWR-1 and 2.5 μM Gö6983. EBs were plated onto gelatin-coated wells and cultured for another 10 days in monolayer before immunostaining.

For differentiation into adipocytes, EBs were formed overnight using Aggrewell™-400 in N2B27 medium alone and then transferred to non-adherent wells for additional 2-day incubation in N2B27 medium plus 5% FBS. Afterwards, EBs were plated onto feeder-coated wells and cultured for another 10 days before staining.

### Oil Red O Staining

Cells were fixed by 10% formalin for 30 min and washed off three times with PBS. Cells were incubated with 60% isopropanol for 5 min, followed by Oil Red O working solution (Sigma-Aldrich) for 20 min on rotator. Oil Red O working solution was then removed, and cells were washed three times with PBS. Cells were then applied with hematoxylin for 1 min and rinsed with PBS 5 times before imaging.

### Immunostaining

Gonadal tissues were fixed in 4% paraformaldehyde at 4° C. overnight and transferred to 30% sucrose until sinking to the bottom of the vial. Afterwards, tissues were embedded in OCT and cryosectioned for immunofluorescence. For staining of attached cells or EBs, cells were fixed in 4% paraformaldehyde for 20 min at room temperature. After fixation, all samples were blocked with PBS containing 5% normal donkey serum and 0.2% Triton X-100 at 4° C. overnight. Samples were then incubated with primary antibodies overnight at 4° C. After washes in PBS with 0.1% Tween-20 three times, secondary antibodies were applied overnight at 4° C. Samples were washed three times in PBS with 0.1% Triton X-100 before mounting for imaging. Nuclei were stained with Hoechst33342 (Invitrogen, 1:50000). Primary antibodies used include the following: Myosin (MF-20, DSHB, 1:50), TuJ-1 (MAB1195, R&D Systems, 1:100), DAZL (ab215718, Abcam, 1:750), P63 (abl24762, Abcam, 1:500), GFP (A11120, Invitrogen, 1:500), SOX9 (AF3075, R&D Systems, 1:40), SSEA-1 (MC480, DSHB, 1:10).

### Ex Ovo Chimera Formation Assay

Albumin agar plates (0.3% w/v agarose, 0.3% w/v glucose with 20 U/ml penicillin and 20 μg/ml) were freshly prepared for avian embryo culture before embryo explant. Embryo was explanted and cultured with a filter paper carrier on albumin agar plate according to the EC culture system. Briefly, freshly laid egg was poured into a petri dish with the thick albumin being removed to expose the embryo directly to the paper ring. Paper was cut around the perimeter of the embryo and embryo was transferred into a dish with PBS to remove any residual yolk. Afterwards, embryo was placed ventral-side up onto albumin agar plate.

For chimera formation assay, GFP-labeled avian ESCs were firstly dissociated by TrypLE for 8 min at RT. After neutralization, these cells were again dissociated by 0.025% Trypsin for 3 min at 38° C. to ensure dissociation into single cells. Then 20,000 GFP-labeled cells were equally distributed on top of the recipient embryo using a glass microcapillary. Embryo was incubated at 38° C. for 2-4 days and donor cell contribution was monitored by live imaging.

### In Ovo Chimera Formation Assay

Surrogate eggshell system was applied to culture chimeric embryos in ovo. If desired, fertile eggs can be irradiated at 500 rads before injection. A surrogate chicken egg 5 to 7 g heavier than the recipient fertile egg was used and the entire content of the recipient fertile egg was transferred into this large surrogate shell. Donor ESCs were dissociated into singles and prepared as described above in the ex ovo chimera formation assay. 7,500 cells were injected into the subgerminal cavity via glass microcapillary in a volume of 0.75 μl. After injection, egg was filled with albumen, covered with cling film and secured using a pair of PVC plastic rings wrapped with rubber bands. The reconstituted egg was placed window-side up on a constantly tilted shaker overnight before being transferred upside down into an incubation cabinet at 38° C. with a rock angle of 45 at half-hour interval. After 4-day incubation, the chimerism of injected embryo can be determined by live imaging based on GFP signal. In order to further incubate the chimeric embryo, a jumbo chicken egg weighing 20 to 25 g heavier than the recipient egg was used as a secondary surrogate shell. The entire content of the chimeric embryo was carefully transferred into this jumbo surrogate shell and sealed with cling film. Chimeric embryo in this jumbo shell was incubated in an incubator at 38° C. until hatchling.

KEY RESOURCES TABLE

| | | |
|---|---|---|
| REAGENT or RESOURCE | SOURCE | IDENTIFIER |

Antibodies

| | | |
|---|---|---|
| Mouse monoclonal | R&D Systems | Cat#MAB1195-SP; |
| anti-beta-III Tubulin | | RRID: AB_357520 |
| Neuron specific (TuJ-1) | | |
| Mouse monoclonal | Developmental Studies Hybridoma | Cat#MF-20; |
| anti-Myosin (MF-20) | Bank (DSHB), University of Iowa, IA | RRID: AB_2147781 |
| Rabbit monoclonal | Abcam | Cat#ab215718; |
| anti-DAZL (EPR21028) | | |
| Mouse monoclonal anti-GFP | Invitrogen | Cat#A-11120; |
| (3E6) | | RRID: AB_221568 |
| Mouse monoclonal | Developmental Studies Hybridoma | Cat#MC-480; |
| anti-SSEA-1 (MC-480) | Bank (DSHB), University of Iowa, IA | RRID: AB_528475 |
| Rabbit monoclonal anti-P63 | Abcam | Cat#ab124762, |
| (EPR5701) | | RRID: AB_10971840 |
| Goat polyclonal anti-SOX9 | R&D Systems | Cat#AF3075, |
| | | RRID: AB_2194160 |
| Goat Anti-Mouse IgM | R&D Systems | Cat#NL019; |
| NorthernLightsTM NL557 | | RRID: AB_10891132 |
| Donkey Anti-Rabbit IgG | Jackson ImmunoResearch | Cat#711-175-152; |
| (H + L)-Cy5-AffiniPure | | RRID: AB_2340607 |
| conjugated | | |
| Donkey Anti-Goat IgG | Jackson ImmunoResearch | Cat#705-175-147; |
| (H + L)- Cy5-AffiniPure | | RRID: AB_2340415 |
| Donkey Anti-Rabbit IgG | Rockland | Cat#611-742-127; |
| (H + L) DyLightTM 549 | | RRID: AB_1057575 |
| Donkey Anti-Mouse | Invitrogen | Cat#A-21202; |
| IgG (H + L), Alexa | | RRID: AB_141607 |
| Fluor 488 Conjugated | | |
| Donkey Anti-Mouse IgG | Rockland | Cat#610-742-124; |
| (H + L) DyLightTM 549 | | RRID: AB_1057571 |

Biological Samples

| | | |
|---|---|---|
| Specific-Pathogen-Free | Charles River | N/A |
| (SPF) fertile chicken | | |
| Fertile Rhode Island Red | AA lab eggs, INC, Westminster, CA | N/A |
| chicken eggs | | |
| Fertile Japanese quail | This paper | N/A |
| (Coturnix japonica) eggs | | |
| Fertile Goose eggs | Sunstate Ranch, Sylmar, CA | N/A |
| Fertile Ostrtich egg | OstrichLand, Solvang, CA | N/A |

Chemicals, Peptides, and Recombinant Proteins

| | | |
|---|---|---|
| IWR-1-endo | Selleck Chemicals | Cat#S7086; CAS: |
| | | 1127442-82-3 |
| Gö6983 | Selleck Chemicals | Cat#S2911; |
| | | CAS: 133053-19-7 |
| SB431542 | Selleck Chemicals | Cat#S1067; |
| | | CAS: 301836-41-9 |
| Chicken Leukemia | Kingfisher Biotech | Cat#RP1395C-100 |
| Inhibitory Factor (LIF) | | |
| (Yeast-derived Recombinant | | |
| Conalbumin from chicken | Sigma-Aldrich | Cat#C7786-100mG; |
| egg white, Ovotransferrin | | CAS: 1391-06-6 |
| N-2 Supplement (100X) | Thermo Fisher Scientific | Cat#17502048 |
| B-27TM Supplement (50X), | Thermo Fisher Scientific | Cat#17504044 |
| serum free | | |
| Oil Red O | Sigma-Aldrich | Cat#O0625-25G |
| Neurobasal Medium | Thermo Fisher Scientific | Cat#21103049 |
| DMEM/F-12 | Thermo Fisher Scientific | Cat#11320033 |
| TrypLETM Express Enzyme | Thermo Fisher Scientific | Cat#12605028 |
| (1X), phenol red | | |
| Cellbanker 2 | Amsbio | Cat#11891 |

Critical Commercial Assays

| | | |
|---|---|---|
| iTaqTM Universal SYBR ® | Bio-Rad | Cat#1725124 |
| Green Supermix | | |
| RNAscope ® 2.5 HD | Advanced Cell Diagnostics | Cat#322300 |
| Assay- BROWN | | |
| RNeasy Mini Kit | Qiagen | Cat#74104 |
| iScriptTM cDNA Synthesis | Bio-Rad | Cat#1708891BUN |

Deposited Data

| | | |
|---|---|---|
| RNA-seq data of | Kalkan et al., 2019 | GEO: GSE121148 |
| transcriptome profiles of | | |
| mouse ES cells in naive and | | |
| formative states | | |
| RNA-seq data of gene | This paper | GEO: |
| expression profiles of | | |
| EGK.X chicken embryos | | |
| and chicken ES cells | | |
| RNA-seq data of | Hwang et al., 2018 | GEO: GSE86592 |
| transcriptome profile of | | |
| chicken embryos at | | |
| EGK.VIII stage | | |
| RNA-seq data of chicken | Weizmann Institute | SRX540095 |
| embryo transcriptome at | | |
| HH4 stage | | |
| RNA-seq data of | Zhang et al., 2016 | GEO: GSE73993 |
| transcriptome profiles of | | |
| mouse ES cells in LIF/KSR | | |
| medium and EpiSCs in | | |
| bFGF/serum-free (KSR) | | |
| medium | | |

Experimental Models: Cell Lines

| | | |
|---|---|---|
| Goose ES cells | This paper | N/A |
| GFP-labeled goose ES cells | This paper | N/A |
| Chicken ES cells derived | This paper | N/A |
| from Rhode Island Red | | |
| GFP-labeled chicken ES | This paper | N/A |
| Irradiated mouse embryonic | This paper | N/A |
| fibroblast | | |
| Quail ES cells | This paper | N/A |
| GFP-labeled quail ES cells | This paper | N/A |

Experimental Models: Organisms/Strains

| | | |
|---|---|---|
| Specific-Pathogen-Free | Charles River | N/A |
| (SPF) fertile chicken | | |

Oligonucleotides

| | | |
|---|---|---|
| chicken Sex genotype | Female&Male: | N/A |
| primers | 12S-F: CTATAATCGATAATCCACGATTCA | |
| | (SEQ ID NO: 1) | |
| | 12S-R: CTTGACCTGTCTTATTAGCGAGG | |
| | (SEQ ID NO: 2) | |
| | Female: | |
| | XhoI-F: CCCAAATATAACACGCTTCACT | |
| | (SEQ ID NO: 3) | |
| | XhoI-R: GAAATGAATTATTTTCTGGCGAC | |
| | (SEQ ID NO: 4) | |

Recombinant DNA

| | | |
|---|---|---|
| pSIN-EF2-EGFP-Puro | Tai et al., 2013 | N/A |

Software and Algorithms

| | | |
|---|---|---|
| Adobe Illustrator | adobe.com/products/illustrator.thml | RRID: SCR_010279 |
| Zen | Zeiss | RRID: SCR_013672 |
| Fiji | fiji.sc/ | RRID: SCR_002285 |
| HCImage Live | Hamamatsu | RRID: SCR_015041 |
| Image Lab | bio-rad.com/en-us/sku/1709690- | RRID: SCR_014210 |
| | image-lab-software | |
| Prism 8 | graphpad.com/ | RRID: SCR_002798 |

Other

| | | |
|---|---|---|
| SZX16 | Olympus | N/A |
| Amicon ® Ultra-15 | Millipore Sigma | Cat#UFC905008 |
| Centrifugal Filter Unit | | |
| Amicon ® Ultra-15 | Millipore Sigma | Cat#UFC910008 |
| Centrifugal Filter Unit | | |
| Acrodisc ® Syringe Filters | PALL | Cat#4654 |
| with Supor ® Membrane, | | |
| Sterile - 0.45 μm, 32 mm | | |
| Acrodisc ® Syringe Filters | PALL | Cat#4652 |
| with Supor ® Membrane, | | |
| Sterile - 0.2 μm, 32 mm | | |
| Amicon Ultra-0.5 | Millipore Sigma | Cat#UFC500324 |
| Centrifugal Filter Unit, | | |
| Ultracel, 3 KDa, 0.5 mL | | |
| BZ X-710 fluorescent | Keyence | Cat# BZ-X710; |
| microscope | | RRID: SCR_017202 |
| Axio Zoom microscope | Zeiss | RRID: SCR_016980 |

### Example 2

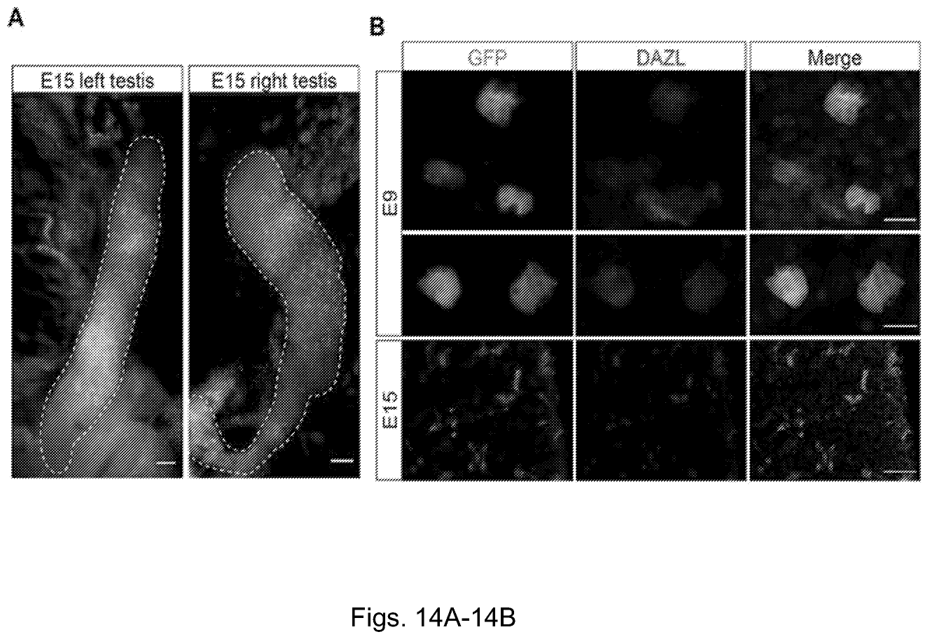

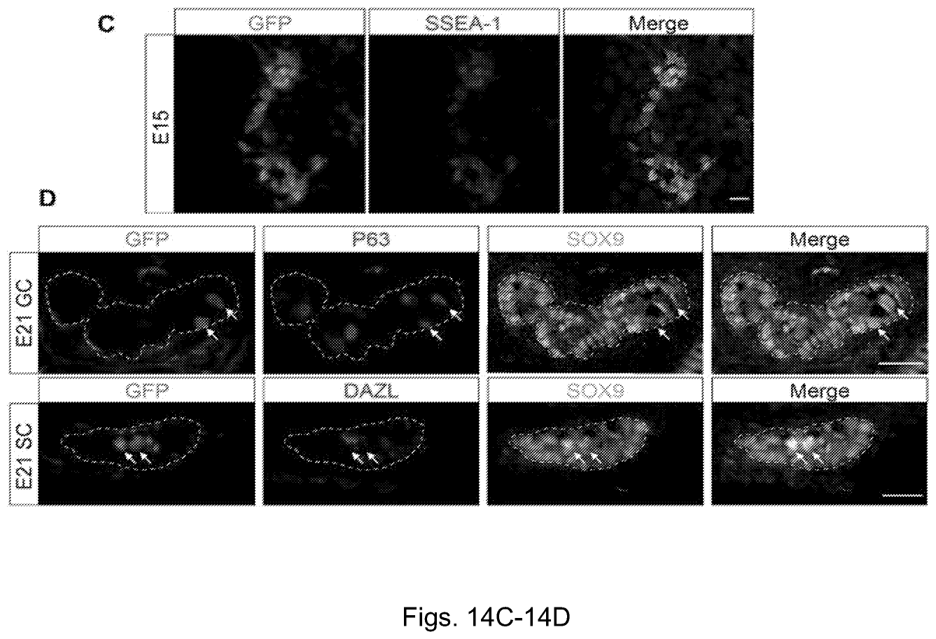

To examine germline contribution in ovo, male GFP-positive chicken ESCs were injected into EGK.X stage chicken embryos pretreated with 500 cGy irradiation. Sublethal irradiation can increase the germline transmission rate of donor PGCs in chicken by reducing the number of endogenous PGCs1. GFP-positive donor cells could be detected inside the gonads of chicken embryos injected with chicken ESCs, especially in high-grade chimeras (FIG. 14A). The presence of ESC-derived germ cells in chimeric embryos was analyzed at different developmental stages. In embryonic day 9 (E9) chimeric embryos, DAZL-positive GFP cells were localized in gonads as small clusters consisting of 1-3 cells (FIG. 14B). In E15 chimeric embryos, as PGCs proliferated, bigger clusters of DAZL-positive GFP cells could be found in gonads and the identity of these donor PGCs was further confirmed by staining with SSEA-1 (FIGS. 14C, 14B), another germ cell marker. In E21 embryos, as gonads continued to develop, PGCs were enclosed inside the seminiferous tubules. GFP-positive PGCs could be detected within the tubules together with endogenous PGCs by staining with P63, a marker for germ cells. Additionally, in some seminiferous tubules, GFP cells also gave rise to Sertoli cells shown by SOX9 staining (FIG. 14D). Notably, the contribution of ESCs into germline lineage inside chicken embryos was confirmed using multiple cell lines derived. In some high-grade chimeras, almost all of the PGCs observed were originated from donor ESCs (FIGS. 14B, 14C). Taken together, through tracing of the germline development of donor chicken ESCs, this demonstrated that chicken ESCs are competent to adopt a germ cell fate both in vitro and in ovo.

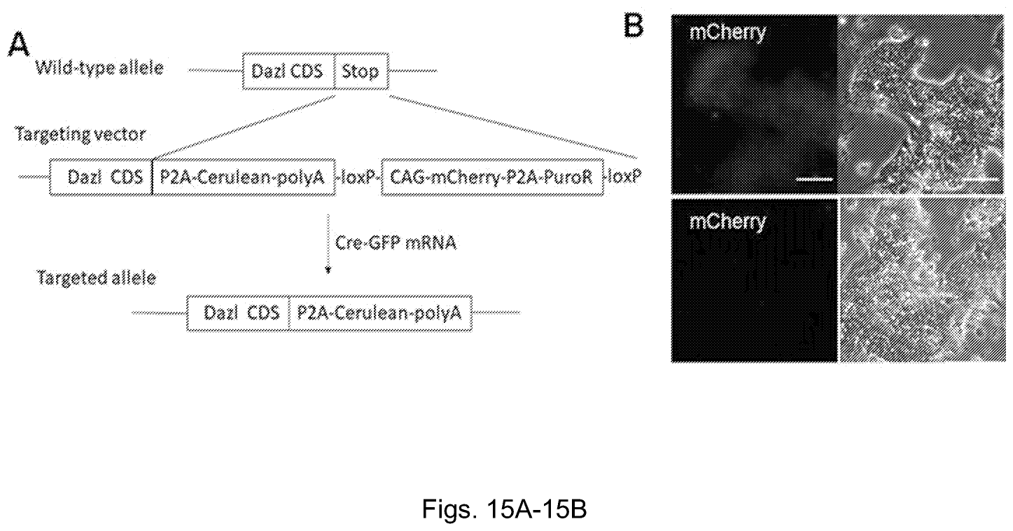

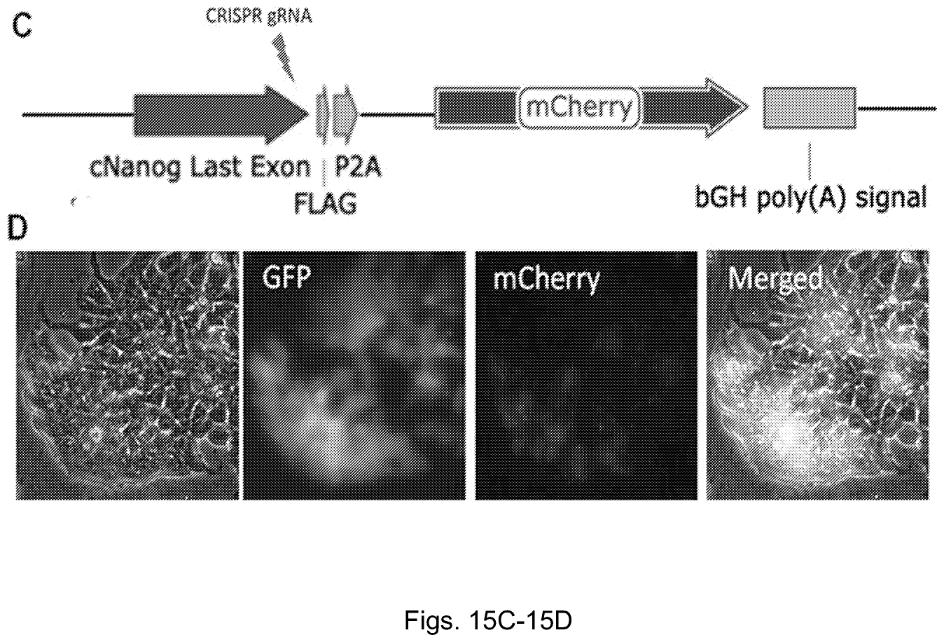

Established herein are Dazl-Cerulean and Nanog-mCherry knockin reporter chicken ESC lines (FIGS. 15A-15D). Dazl-Cerulean is expected to specifically express in germ cells while both undifferentiated chicken ESCs and PGCs derived from chicken ESCs should express Nanog-mCherry.

According to the “The U.S. State of the Birds” report (available online at stateofthebirds.org/2019/), one-third of US bird species are currently endangered. There is an urgent need to develop strategies to save these endangered bird species. iPSCs can be generated from somatic cells and are capable of differentiation into germ cells, therefore holding great promise for preserving endangered species and reviving extinct species².

Successful generation of iPSCs depends on 1) the development of methods for the maintenance of pluripotent stem cells; 2) the identification of transcription factors that can reprogram somatic cells to become pluripotent stem cells; and 3) the methods used to introduce the transcription factors into somatic cells. Described herein are the transcription factors that are necessary to reprogram chicken embryonic fibroblasts to iPSCs.

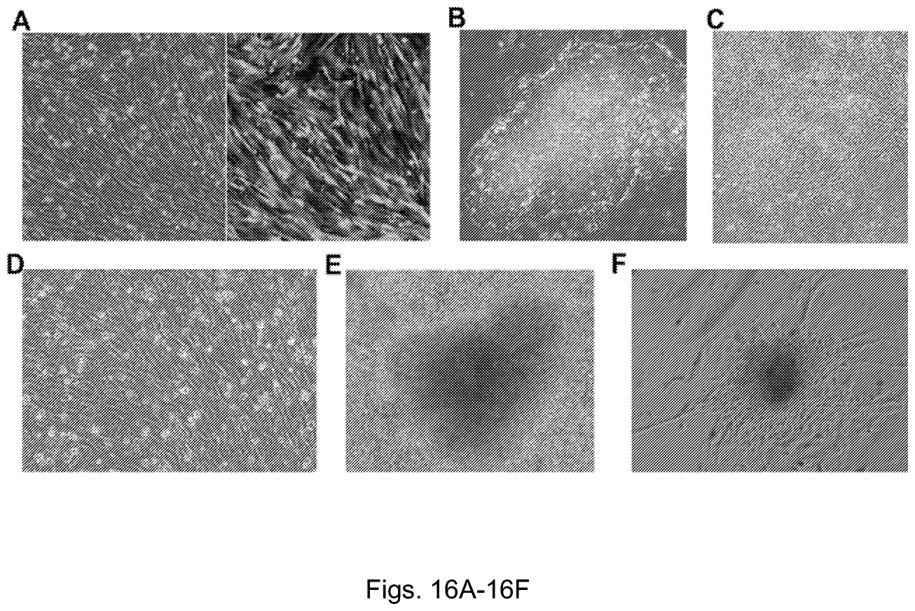

The four transcription factors, Oct4, Sox2, Klf4, and c-Myc (OSKM) have been used to successfully generate authentic iPSCs from mice, rat, and human³⁻⁶. The establishment of culture conditions for the derivation and maintenance of ESCs is necessary for the successful generation of authentic iPSCs from the same species. Based on the condition described herein developed for chicken ESCs, it was tested whether OSKM is sufficient to reprogram chicken embryonic fibroblasts (CEFs) into iPSCs. CEFs were derived from RIR chicken embryos and it was demonstrated that CEFs could be efficiently transduced using retroviral vector with GFP (FIG. 16A). iPSCs could be generated from mouse embryonic fibroblasts (MEFs) by mouse OSKM (FIG. 16B), but not chicken OSKM (FIG. 16C). No colony emerged in CEFs transduced with retroviral vectors carrying mouse OSKM (FIG. 16D). In contrast, many iPSC-like colonies appeared in CEFs transduced with chicken OSKM (FIG. 16E), indicating that the use of species-specific transcription factors is critical for reprogramming. These chicken iPSCs can efficiently differentiate into neurons (FIG. 16F).

RNA-seq data generated from chicken ESCs and CEFs was compared and the following top 20 transcription factor candidates that are highly and specifically expressed in chicken ESCs were identified: Pou5f3 (Oct4), Sox3, Nanog, Noto, Cdx2, Klf4, Lin28B, Gnot2, Gata4, Vgll1, Vgll2, Hand1, Sp5, Otx2, Dbx2, Dmrt3, Tfap2A, Sp6, Eomes, and Tbx3. It was found that replacing chicken Sox2 in the chicken OSKM with chicken Sox3 can further improve chicken iPSC derivation.

### REFERENCES

1 Nakamura, Y. et al. X-irradiation removes endogenous primordial germ cells (PGCs) and increases germline transmission of donor PGCs in chimeric chickens. J Reprod Dev 58, 432-437, doi: 10.1262/jrd.2012-045 (2012).
2 Stanton, M. M. et al. Prospects for the Use of Induced Pluripotent Stem Cells in Animal Conservation and Environmental Protection. Stem Cells Transl Med 8, 7-13, doi: 10.1002/sctm.18-0047 (2019).
3 Hamanaka, S. et al. Generation of germline-competent rat induced pluripotent stem cells. PLoS One 6, e22008, doi:10.1371/journal.pone.0022008 (2011).
4 Yu, J. et al. Induced pluripotent stem cell lines derived from human somatic cells. Science 318, 1917-1920, doi:10.1126/science.1151526 (2007).
5 Takahashi, K. et al. Induction of pluripotent stem cells from adult human fibroblasts by defined factors. Cell 131, 861-872, doi:10.1016/j.cell.2007.11.019 (2007).
6 Takahashi, K. & Yamanaka, S. Induction of pluripotent stem cells from mouse embryonic and adult fibroblast cultures by defined factors. Cell 126, 663-676, doi:10.1016/j.cell.2006.07.024 (2006).

1. A method of producing, maintaining, or expanding oviparous stem cells, the method comprising:
contacting at least one oviparous stem cell or oviparous blastodermal cell with at least one of:
a Wnt inhibitor; a protein kinase C (PKC) inhibitor; and ovotransferrin.

2. The method of claim 1, wherein the contacting comprises contacting the at least one oviparous stem cell or oviparous blastodermal cell with a cell culture medium comprising at least one of: a Wnt inhibitor; a protein kinase C (PKC inhibitor); and ovotransferrin.

3. The method of claim 1, wherein the contacting comprises contacting the at least one oviparous stem cell or avian blastodermal cell with a cell culture medium comprising at least one of: a Wnt inhibitor; a protein kinase C (PKC inhibitor); and egg extract comprising ovotransferrin.

4. The method of claim 1,
wherein the at least one oviparous stem cell or oviparous blastodermal cell is contacted with:
a) a Wnt inhibitor and a PKC inhibitor;
b) a Wnt inhibitor and ovotransferrin;
c) a PKC inhibitor and ovotransferrin; or
d) a Wnt inhibitor; a PKC inhibitor; and ovotransferrin.

5.-7. (canceled)

8. The method of claim 1,
wherein the Wnt inhibitor is selected from the group consisting of:
IWR-1, E7449, AZ6102, WIKI4, 53AH, C59, XAV-939, ICG-001, triptonide, IWP-2, M2912 (MSC2504877), CCT251545, KY02111, M2912, Box5, KY05009, Heparan sulfate, Tetramethylthiuram disulfide (TMTD), JW55, MN64, RK-287107, G007-LK, and adavivint (SM04690).

9. The method of claim 1,
wherein the Wnt inhibitor is IWR-1.

10. The method of claim 1,
wherein the PKC inhibitor is selected from the group consisting of:
Go6983, Go7874, Go6976, LY317615, GSK590693, HA-1077, Ro320432, Calophostin C, AEB071 (sotrastaurin), NSC-301739 (mitoxantrone), AM-2282 (staurosporine), GF109203X (bisindolylmaleimide I), Ro 31-8220 (bisindolylmaleimide IX), daphnetin, dequalinium chlrodie, A-3 hydrochloride, N-desmethyltamoxifen, bisindolylmaleimide VIII, H-1152, HA-100, rottlerin, bisindolylmaleimide IV, VTX-27, valrubicin, PKC-theta inhibitor, ML-7, LY333531, PKC412, NSC 646662, WAY-301398, epsilon-VI-2, chelerythrine, hypocrellin A, hypericin, oncrasin-1, LXS-196, NAS-301, 2-methoxy-1,4-naphthoquinone, quercetin, myricitrin, and methyl-hesperidin.

11. The method of claim 1,
wherein the PKC inhibitor is Go6983.

12. The method of claim 1,
wherein the ovotrasferrin is an oviparous ovotransferrin.

13.-20. (canceled)

21. The method of claim 1,
wherein the at least one oviparous stem cell or oviparous blastodermal cell is further contacted with an activin receptor-like kinase 4, -5, and -7 inhibitor.

22.-23. (canceled)

24. The method of claim 1,
wherein the at least one oviparous stem cell or oviparous blastodermal cell is further contacted with a LIF polypeptide.

25.-35. (canceled)

36. The method of claim 1,
wherein the at least one oviparous stem cell or oviparous blastodermal cell is further contacted with egg yolk or egg extract ex ovo.

37.-39. (canceled)

40. The method of claim 1, wherein the oviparous stem cells or at least one oviparous stem cell is an embryonic stem cell (ESC), an induced pluripotent stem cell (iPSC), multipotent stem cell, pluripotent stem cell, a reprogrammed somatic cell, or reprogrammed extra-embryonic cell.

41.-70. (canceled)

71. A composition or combination comprising:
at least one Wnt inhibitor;
at least one protein kinase C (PKC) inhibitor; and
ovotransferrin.

72.-118. (canceled)

119. A method of producing an avian induced pluripotent stem cell, the method comprising contacting an oviparous cell with one or more of Oct 4, Sox2, Sox3, Klf4, c-Myc, Nanog, Noto, Cdx2, Lin28B, Gnot2, Gata4, Vgll1, Vgll2, Hand 1, Sp5, Otx2, Dbx2, Dmrt3, Tfap2A, Sp6, Eomes, and Tbx3.

120.-123. (canceled)

124. A stem cell produced, maintained, or expanded according to claim 1.

125. A method of veterinary treatment, the method comprising administering a stem cell produced, maintained, or expanded according to claim 1, or a cell differentiated from the stem cell, to a non-human animal.

126. (canceled)

127. (canceled)

128. A method of biomanufacturing, the method comprising purifying or collecting a biomaterial produced by a stem cell produced, maintained, or expanded according to claim 1, or from a cell differentiated from the stem cell.

129.-134. (canceled)

135. A method of somatic cell nuclear transfer, the method comprising transferring nuclear material from a stem cell produced, maintained, or expanded according to claim 1, to a somatic cell.

136. method of producing a lineage restricted cell, the method comprising differentiating a stem cell produced, maintained, or expanded according to claim 1 to a lineage restricted cell.

137.-142. (canceled)


SEQUENCE LISTING (6 sequences)

SEQ ID NO: 1 - protein, 686 aa, Gallus gallus
APPKSVIRWCTISSPEEKKCNNLRDLTQQERISLTCVQKATYLDCIKAIANNEADAISLDGGQVFEAGLAPYKLKPIAAEVYEHTEGSTTSYYAVAVVKKGTEFTVNDLQGKTSCHTGLGRSAGWNIPIGTLIHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDSKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPERCVASSHEKYFGYTGALRCLVEKGDVAFVQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 2 - protein, 211 aa, Gallus gallus
MRLIPAGVVPFVALLLLQRRPVSGRALLGTSSACPTNGLCRANVLEQTRRQVALLNATAQDLFSLYLKCQGEPFSSESDRLCSPSGIFFPPFHVNRTTERKEVMVAMYKLFAFLNASLGNITRDQEELNPMAKELLNRLHNTTKTTRGLISNLTCLLCKHYNIFQVDVSYGESSKDKSAFKKKQQGCQVLRKYVQVIAQAARVLLPHLSPA

SEQ ID NO: 3 - DNA, 22 bp, synthetic construct
  source 1..22: mol_type = other DNA
cccaaatataacacgcttcact

SEQ ID NO: 4 - DNA, 23 bp, synthetic construct
  source 1..23: mol_type = other DNA
gaaatgaattattttctggcgac

SEQ ID NO: 5 - DNA, 24 bp, synthetic construct
  source 1..24: mol_type = other DNA
ctataatcgataatccacgattca

SEQ ID NO: 6 - DNA, 23 bp, synthetic construct
  source 1..23: mol_type = other DNA
cttgacctgtcttattagcgagg